(19) United States
(12) Patent Application Publication
(10) Pub. No.: US 2021/0261528 A1
(43) Pub. Date: Aug. 26, 2021

(54) SALTS OF (S)-(5-CYCLOBUTOXY-2-METHYL-6-(1-(PIPERIDIN-4-YL)-1H-PYRAZOL-4-YL)-3,4-DIHYDROQUINOLIN-1(2H)-YL)(CYCLOPROPYL)METHANONE AND SOLID FORMS THEREOF

(71) Applicant: FORMA Therapeutics, Inc., Watertown, MA (US)

(72) Inventor: George P. Luke, Clinton, CT (US)

(73) Assignees: FORMA Therapeutics, Inc., Watertown, MA (US); FORMA Therapeutics, Inc., Watertown, MA (US)

(21) Appl. No.: 17/255,054

(22) PCT Filed: Jun. 28, 2019
(86) PCT No.: PCT/US2019/039677

§ 371 Date: Dec. 22, 2020

Related U.S. Application Data
(60) Provisional application No. 62/692,546, filed on Jun. 29, 2018.
(60) Provisional application No. 62/692,554, filed on Jun. 29, 2018.

(51) Int. Cl. C07D 401/14

(52) U.S. Cl. CPC C07D 401/14; C07B 2200/13

(57) ABSTRACT

The present disclosure reports salts of (S)-(5-cyclobutoxy-2-methyl-6-(1-(piperidin-4-yl)-1H-pyrazol-4-yl)-3,4-dihydroquinolin-1(2H)-yl)(cyclopropyl)methanone, solid forms thereof, and methods of making and using the same.

### CROSS REFERENCE TO RELATED APPLICATIONS

The present application claims priority to U.S. Provisional Patent Application No. 62/692,546, filed Jun. 29, 2018 and U.S. Provisional Patent Application No. 62/692,554, filed Jun. 29, 2018, the entire contents of each of which are hereby incorporated by reference.

### TECHNICAL FIELD

The present disclosure relates to pharmaceutical compositions, including salts of a certain compound, and solid forms thereof, useful for inhibiting bromo and extra terminal (BET) bromodomains.

### BACKGROUND

Chemical compounds can form one or more different pharmaceutically acceptable salts and/or solid forms, including amorphous and polymorphic crystal forms. Individual salts and solid forms of bioactive chemical compounds can have different properties. There is a need for the identification and selection of appropriate salts and/or solid forms of bioactive chemical compounds (including appropriate crystalline forms, where applicable) for the development of pharmaceutically acceptable dosage forms for the treatment of various diseases or conditions.

The bioactive compound (S)-(5-cyclobutoxy-2-methyl-6-(1-(piperidin-4-yl)-1H-pyrazol-4-yl)-3,4-dihydroquinolin-1(2H)-yl)(cyclopropyl)methanone (“Compound 1”)

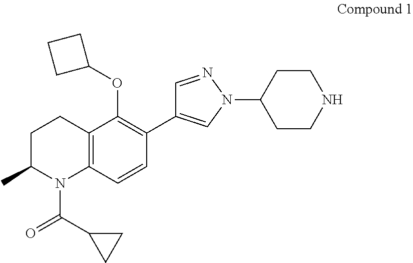 [SMILES: C[C@H]1CCc2c(ccc(-c3cnn(C4CCNCC4)c3)c2OC2CCC2)N1C(=O)C1CC1]

is a small molecule modulator of bromo and extra terminal (BET) bromodomains. Compound 1 is disclosed in PCT Application Publication No. WO 2015/074064 as one of many compounds suitable as small molecule modulators of BET bromodomains. Clinical trials under NCT02543879, entitled “Study of a Novel BET Inhibitor FT-1101 in Patients With Relapsed or Refractory Hematologic Malignancies,” disclose the use of BET inhibitor FT-1101 in patients with relapsed or refractory hematologic malignancies. There remains a need for identifying salt forms and solid forms of Compound 1 useful for various therapeutic applications.

### SUMMARY

Salts and other solid forms of Compound 1 disclosed herein include Compound 1 as a fumarate salt (including crystalline fumarate salt Form A, Form B, and Form C), an adipate salt (including crystalline adipate salt Form A), and a succinate salt (i.e., Compound 2, which includes crystalline succinate salt Form A, Form B, Form C, Form D, Form E, Form F, Form G, Form I, Form K, Form L, Form M, Form O, and Form P). The present disclosure provides various solid forms of Compound 1, including one or more pharmaceutically acceptable salts forms for Compound 1 useful for the therapeutic oral administration of Compound 1. Certain salt forms of Compound 1 form crystalline solid forms. The various solid forms of Compound 1 can be identified by certain characteristic properties. For example, certain crystalline forms of the salts of Compound 1 have distinct characteristic XRPD peaks (see Example 3) that are not reported in previously reported forms of Compound 1.

A novel Compound 1 crystalline fumarate salt Form A can be identified by X-ray Powder Diffraction (XRPD) pattern having one or more characteristic diffractions at angles (2 theta±0.2) of 4.0, 5.3, 7.9, 10.2, 13.3, and 21.2.

A novel Compound 1 crystalline fumarate salt Form B can be identified by X-ray Powder Diffraction (XRPD) pattern having one or more characteristic diffractions at angles (2 theta±0.2) of 5.4, 13.5, 16.3, 21.1, 23.5, 26.0, and 26.5.

A novel Compound 1 crystalline fumarate salt Form C can be identified by X-ray Powder Diffraction (XRPD) pattern having one or more characteristic diffractions at angles (2 theta±0.2) of 5.5, 8.2, 10.0, 18.1, 19.2, and 22.0.

A novel Compound 1 crystalline adipate salt Form A can be identified by X-ray Powder Diffraction (XRPD) pattern having one or more characteristic diffractions at angles (2 theta±0.2) of 7.9, 12.3, 15.7, 19.7, and 24.7.

A novel Compound 1 crystalline succinate salt Form A can be identified by X-ray Powder Diffraction (XRPD) pattern having one or more characteristic diffractions at angles (2 theta±0.2) of 5.5, 8.3, 9.8, 17.9, 22.2, 24.8, 25.6, 36.1, and 37.3.

Applicant has also recognized that novel Compound 1 salt forms can be obtained by treating Compound 1 with an acid selected from fumaric acid, adipic acid, and succinic acid.

In some embodiments, the present disclosure provides novel solid forms of (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate (“Compound 2”), including crystalline forms of Compound 2. The present disclosure is based in part on the identification of various solid forms of Compound 2. In particular, Applicants have recognized that Compound 2 forms various crystalline solid forms, including one or more pharmaceutically acceptable crystalline forms of Compound 2 that are useful for the therapeutic oral administration of (S)-(5-cyclobutoxy-2-methyl-6-(1-(piperidin-4-yl)-1H-pyrazol-4-yl)-3,4-dihydroquinolin-1(2H)-yl)(cyclopropyl)methanone (i.e., Compound 1). The various crystalline solid forms of Compound 2 can be identified by certain characteristic properties. Certain crystalline forms of Compound 2 have distinct characteristic XRPD peaks (see Example 9) that are not reported in previously reported forms of Compound 1. For example, the present disclosure provides Compound 2 in various solid forms designated herein as: Form A, Form B, Form C, Form D, Form E, Form F, Form G, Form I, Form K, Form L, Form M, Form O, and Form P, as well as compositions comprising a solid form of Compound 2 comprising one or more of Form A, Form B, Form C, Form D, Form E, Form F, Form G, Form I, Form K, Form L, Form M, Form O, and Form P.

A novel Compound 2 Form A can be identified by X-ray Powder Diffraction (XRPD), having one or more characteristic diffractions at angles (2 theta±0.2) of 5.5, 8.3, 9.8, 17.9, 22.2, 24.8, 25.6, 36.1, and 37.3.

A novel Compound 2 Form B can be identified by X-ray Powder Diffraction (XRPD), having one or more characteristic diffractions at angles (2 theta±0.2) of 4.7, 5.9, 8.7, 11.0, 17.2, and 20.4.

A novel Compound 2 Form C can be identified by X-ray Powder Diffraction (XRPD), having one or more characteristic diffractions at angles (2 theta±0.2) of 5.4, 8.1, 10.1, 10.9, 16.4, 17.7, and 34.3.

A novel Compound 2 Form D can be identified by X-ray Powder Diffraction (XRPD), having one or more characteristic diffractions at angles (2 theta±0.2) of 5.4, 14.7, 18.5, 21.8, and 38.6.

A novel Compound 2 Form E can be identified by X-ray Powder Diffraction (XRPD), having one or more characteristic diffractions at angles (2 theta±0.2) of 5.5, 11.3, 13.0, 16.3, and 20.3.

A novel Compound 2 Form F can be identified by X-ray Powder Diffraction (XRPD), having one or more characteristic diffractions at angles (2 theta±0.2) of 13.6, 14.2, 16.3, 21.8, and 26.7.

A novel Compound 2 Form G can be identified by X-ray Powder Diffraction (XRPD), having one or more characteristic diffractions at angles (2 theta±0.2) of 4.4, 6.8, 9.1, 15.3, and 38.8.

A novel Compound 2 Form I can be identified by X-ray Powder Diffraction (XRPD), having one or more characteristic diffractions at angles (2 theta±0.2) of 3.4, 5.9, 9.2, 10.3, 11.0, and 25.4.

A novel Compound 2 Form J can be identified by X-ray Powder Diffraction (XRPD), having one or more characteristic diffractions at angles (2 theta±0.2) of 5.4, 8.1, 12.4, 13.5, 15.6, 21.0, and 21.7.

A novel Compound 2 Form K can be identified by X-ray Powder Diffraction (XRPD), having one or more characteristic diffractions at angles (2 theta±0.2) of 5.5, 8.7, 9.7, 22.2, and 24.4.

A novel Compound 2 Form L can be identified by X-ray Powder Diffraction (XRPD), having one or more characteristic diffractions at angles (2 theta±0.2) of 5.5, 9.8, 12.6, 17.8, 18.9, 21.0, 22.2, and 29.2.

A novel Compound 2 Form M can be identified by X-ray Powder Diffraction (XRPD), having one or more characteristic diffractions at angles (2 theta±0.2) of 5.5, 8.3, 14.5, 19.4, 22.1, 24.9, and 37.7.

A novel Compound 2 Form O can be identified by X-ray Powder Diffraction (XRPD), having one or more characteristic diffractions at angles (2 theta±0.2) of 3.4, 4.6, 6.8, 9.2, 10.1, 10.9, 12.0, and 20.2.

A novel Compound 2 Form P can be identified by X-ray Powder Diffraction (XRPD), having one or more characteristic diffractions at angles (2 theta±0.2) of 4.3, 6.8, 12.3, 15.2, and 39.6.

The Applicant has also recognized that novel Compound 2 solid forms (e.g., Form A, Form B, Form C, Form D, Form E, Form F, Form G, Form I, Form J, Form K, Form M, Form O, and Form P) can be obtained by maintaining a Compound 2 solid form under physical conditions effective to convert Compound 2 in a first solid form into Compound 2 in a second solid form.

### BRIEF DESCRIPTION OF THE DRAWINGS

FIG. 1 depicts an X-ray Powder Diffraction (XRPD) pattern of Compound 1 Fumarate Form A and an XRPD pattern of Fumaric Acid. The upper pattern corresponds to Compound 1 Fumarate Form A. The lower pattern corresponds to Fumaric Acid.

FIG. 2 is a thermogravimetric analysis (TGA) curve (upper curve) and a differential scanning calorimetry (DSC) thermogram (lower curve) for Compound 1 Fumarate Form A.

FIG. 3 depicts an XRPD pattern of Compound 1 Fumarate Form B and an XRPD pattern of Fumaric Acid. The upper pattern corresponds to Compound 1 Fumarate Form B. The lower pattern corresponds to Fumaric Acid.

FIG. 4 is a thermogravimetric analysis (TGA) curve (upper curve) and a differential scanning calorimetry (DSC) thermogram (lower curve) for Compound 1 Fumarate Form B.

FIG. 5 depicts an XRPD pattern of Compound 1 Fumarate Form C and an XRPD of Fumaric Acid. The upper pattern corresponds to Compound 1 Fumarate Form C. The lower pattern corresponds to Fumaric Acid.

FIG. 6 is a thermogravimetric analysis (TGA) curve (upper curve) and a differential scanning calorimetry (DSC) thermogram (lower curve) for Compound 1 Fumarate Form C.

FIG. 7 depicts an XRPD of Compound 1 Adipate Form A and an XRPD of Adipic Acid. The upper pattern corresponds to Compound 1 Adipate Form A. The lower pattern corresponds to Adipic Acid.

FIG. 8 is a thermogravimetric analysis (TGA) curve (upper curve) and a differential scanning calorimetry (DSC) thermogram (lower curve) for Compound 1 Adipate Form A.

FIG. 9 depicts an XRPD pattern of one sample of Compound 1 Succinate Form A (i.e., Compound 2 Form A) and an XRPD of Succinic Acid. The upper pattern corresponds to Compound 1 Succinate Form A (i.e., Compound 2 Form A). The lower pattern corresponds to Succinic Acid.

FIG. 10 depicts an XRPD pattern of another sample of Compound 2 Form A (i.e., Compound 1 Succinate Form A).

FIG. 11 is a thermogravimetric analysis (TGA) curve (upper curve) and a differential scanning calorimetry (DSC) thermogram (lower curve) for one sample of Compound 1 Succinate Form A (i.e., Compound 2 Form A).

FIG. 12 is a thermogravimetric analysis (TGA) curve (upper curve) and a differential scanning calorimetry (DSC) thermogram (lower curve) for another sample of Compound 2 Form A (i.e., Compound 1 Succinate Form A).

FIG. 13 depicts an XRPD pattern of Compound 1 Maleate Form A and an XRPD pattern of Maleic Acid. The upper pattern corresponds to Compound 1 Maleate Form A. The lower pattern corresponds to Maleic Acid.

FIG. 14 is a differential scanning calorimetry (DSC) thermogram for Compound 1 Maleate Form A.

FIG. 15 is a dynamic vapor sorption (DVS) analysis of Compound 1 Fumarate Form A.

FIG. 16 is a dynamic vapor sorption (DVS) analysis of Compound 1 Fumarate Form B.

FIG. 17 is a dynamic vapor sorption (DVS) analysis of Compound 1 Fumarate Form C.

FIG. 18 is a dynamic vapor sorption (DVS) analysis of Compound 1 Adipate Form A.

FIG. 19 is a dynamic vapor sorption (DVS) analysis of Compound 1 Succinate Form A (i.e., Compound 2 Form A).

FIG. 20 is a series of XRPD patterns depicting the results of slurry conversions of Compound 1 Fumarate Form A, Form B, and Form C.

FIG. 21 is a series of XRPD patterns depicting the results of the recrystallization of Compound 1 Adipate Form A.

FIG. 22 depicts a series of X-ray Powder Diffraction (XRPD) patterns of Compound 2 Form A, Form B, Form C, Form D, Form E, and Form F.

FIG. 23 depicts a series of XRPD patterns of Compound 2 Form G, Form I, Form J, Form K, Form L, and Form M.

FIG. 24 depicts an XRPD pattern of Compound 2 Form B.

FIG. 25 is a thermogravimetric analysis (TGA) curve (upper curve) and a differential scanning calorimetry (DSC) thermogram (lower curve) for Compound 2 Form B.

FIG. 26 depicts a Dynamic Vapor Sorption (DVS) plot of Compound 2 Form B.

FIG. 27 depicts an XRPD pattern of Compound 2 Form C.

FIG. 28 is a thermogravimetric analysis (TGA) curve (upper curve) and a differential scanning calorimetry (DSC) thermogram (lower curve) for Compound 2 Form C.

FIG. 29 depicts an XRPD pattern of Compound 2 Form D.

FIG. 30 is a thermogravimetric analysis (TGA) curve (upper curve) and a differential scanning calorimetry (DSC) thermogram (lower curve) for Compound 2 Form D.

FIG. 31 depicts an XRPD pattern of Compound 2 Form E.

FIG. 32 is a thermogravimetric analysis (TGA) curve (upper curve) and a differential scanning calorimetry (DSC) thermogram (lower curve) for Compound 2 Form E.

FIG. 33 depicts an XRPD pattern of Compound 2 Form F.

FIG. 34 is a thermogravimetric analysis (TGA) curve (upper curve) and a differential scanning calorimetry (DSC) thermogram (lower curve) for Compound 2 Form F.

FIG. 35 depicts an XRPD pattern of Compound 2 Form G.

FIG. 36 is a thermogravimetric analysis (TGA) curve (upper curve) and a differential scanning calorimetry (DSC) thermogram (lower curve) for Compound 2 Form G.

FIG. 37 depicts a Dynamic Vapor Sorption (DVS) plot of Compound 2 Form G.

FIG. 38 depicts an image obtained from polarized light microscopy of Compound 2 Form G.

FIG. 39 depicts an XRPD pattern of Compound 2 Form I.

FIG. 40 is a thermogravimetric analysis (TGA) curve (upper curve) and a differential scanning calorimetry (DSC) thermogram (lower curve) for Compound 2 Form I.

FIG. 41 depicts a Dynamic Vapor Sorption (DVS) plot of Compound 2 Form I.

FIG. 42 depicts an XRPD pattern of Compound 2 Form J.

FIG. 43 is a thermogravimetric analysis (TGA) curve (upper curve) and a differential scanning calorimetry (DSC) thermogram (lower curve) for Compound 2 Form J.

FIG. 44 depicts an XRPD pattern of Compound 2 Form K.

FIG. 45 is a thermogravimetric analysis (TGA) curve (upper curve) and a differential scanning calorimetry (DSC) thermogram (lower curve) for Compound 2 Form K.

FIG. 46 depicts an XRPD pattern of Compound 2 Form L.

FIG. 47 is a thermogravimetric analysis (TGA) curve (upper curve) and a differential scanning calorimetry (DSC) thermogram (lower curve) for Compound 2 Form L.

FIG. 48 depicts an XRPD pattern of Compound 2 Form M.

FIG. 49 is a thermogravimetric analysis (TGA) curve (upper curve) and a differential scanning calorimetry (DSC) thermogram (lower curve) for Compound 2 Form M.

FIG. 50 depicts a Dynamic Vapor Sorption (DVS) plot of Compound 2 Form M.

FIG. 51 depicts an XRPD pattern of Compound 2 Form O.

FIG. 52 is a thermogravimetric analysis (TGA) curve (upper curve) and a differential scanning calorimetry (DSC) thermogram (lower curve) for Compound 2 Form O.

FIG. 53 depicts a Dynamic Vapor Sorption (DVS) plot of Compound 2 Form O.

FIG. 54 depicts an XRPD pattern of Compound 2 Form P.

FIG. 55 is a flow chart illustrating methods of preparing Compound 2 Form G from Compound 2 Form B, Form I, or Form O.

FIG. 56 depicts a series of XRPD patterns from the results of a slurry competition between Compound 2 Form G and Form O.

FIG. 57 depicts a series of XRPD patterns from the results of a stability evaluation of Compound 2 Form G, Form B, and Form I.

FIG. 58 depicts a series of XRPD patterns illustrating the critical a_{w} between Compound 2 Form G and Form I (for a_{w} 0, 0.20, 0.40, 0.59, and 0.80).

FIG. 59 depicts a series of XRPD patterns illustrating the critical a_{w} between Compound 2 Form G and Form I (for a_{w} 0, 0.18, 0.36, and 0.55).

FIG. 60 depicts a series of XRPD patterns illustrating the results of a solubility comparison between Compound 2 Form A and Form G.

FIG. 61 depicts a series of XRPD patterns illustrating the stability of Compound 2 Form G under varying conditions.

### DETAILED DESCRIPTION

The bioactive chemical compound (S)-(5-cyclobutoxy-2-methyl-6-(1-(piperidin-4-yl)-1H-pyrazol-4-yl)-3,4-dihydroquinolin-1(2H)-yl)(cyclopropyl)methanone (“Compound 1”):

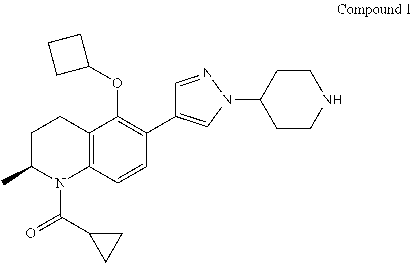 [SMILES: C[C@H]1CCc2c(ccc(-c3cnn(C4CCNCC4)c3)c2OC2CCC2)N1C(=O)C1CC1]

is a small molecule modulator of bromo and extra terminal (BET) bromodomains. The present disclosure provides various salt forms of Compound 1, solid forms thereof, pharmaceutical compositions thereof, and methods of preparing those novel salt forms of Compound 1 and solid forms thereof. Salt forms and solid forms (e.g., crystalline solid forms) impart or may impart characteristics such as improved solubility, stability, and ease of formulation. As used herein, unless otherwise indicated the term “salt” refers to salts or co-crystals of two or more (e.g., two) component molecules (e.g., Compound 1 and a co-former).

### Salt Forms of Compound 1

In some embodiments, novel salt forms of Compound 1 are fumarate salt forms (i.e., “Compound 1 Fumarate”). In some embodiments, Compound 1 Fumarate is amorphous. In some embodiments, Compound 1 Fumarate is a crystalline salt form. In some embodiments, Compound 1 Fumarate is a crystalline salt Form A (i.e., “Compound 1 Fumarate Form A”). In some embodiments, Compound 1 Fumarate is a crystalline salt Form B (i.e., “Compound 1 Fumarate Form B”). In some embodiments, Compound 1 Fumarate is a crystalline salt Form C (i.e., “Compound 1 Fumarate Form C”).

In some embodiments, novel salt forms of Compound 1 are adipate salt forms (i.e., “Compound 1 Adipate”). In some embodiments, Compound 1 Adipate is amorphous. In some embodiments, Compound 1 Adipate is a crystalline salt form. In some embodiments, Compound 1 Adipate is a crystalline salt Form A (i.e., “Compound 1 Adipate Form A”).

In some embodiments, novel salt forms of Compound 1 are succinate salt forms (i.e., “Compound 1 Succinate” or “Compound 2”). In some embodiments, Compound 1 Succinate (i.e., Compound 2) is amorphous. In some embodiments, Compound 1 Succinate (i.e., Compound 2) is a crystalline salt form. In some embodiments, Compound 1 Succinate (i.e., Compound 2) is a crystalline salt Form A (i.e., “Compound 1 Succinate Form A” or “Compound 2 Form A”).

In some embodiments, novel salt forms of Compound 1 are maleate salt forms (i.e., “Compound 1 Maleate”). In some embodiments, Compound 1 Maleate is amorphous. In some embodiments, Compound 1 Maleate is a crystalline salt form. In some embodiments, Compound 1 Maleate is a crystalline salt Form A (i.e., “Compound 1 Maleate Form A”). Novel salt forms of Compound 1 can be obtained by a variety of methods known to those of skill in the art. Suitable methods of preparation are reported in Example 3. For example, in some embodiments, novel salt forms of Compound 1 (e.g., Compound 1 Fumarate, Compound 1 Adipate, and Compound 1 Succinate) can be obtained by treating Compound 1 with an acid selected from fumaric acid, adipic acid, and succinic acid.

Novel salt forms of Compound 1 can be identified by X-ray Powder Diffraction (XPRD). The novel salt forms of Compound 1 disclosed herein include Compound 1 Fumarate (including amorphous Compound 1 Fumarate, Compound 1 Fumarate Form A, Compound 1 Fumarate Form B, Compound 1 Fumarate Form C), Compound 1 Adipate (including amorphous Compound 1 Adipate and Compound 1 Adipate Form A), and/or Compound 1 Succinate (including amorphous Compound 1 Succinate and Compound 1 Succinate Form A (i.e., Compound 2 Form A) and Compound 2 Form B, Compound 2 Form C, Compound 2 Form D, Compound 2 Form E, Compound 2 Form F, Compound 2 Form G, Compound 2 Form I, Compound 2 Form K, Compound 2 Form L, Compound 2 Form M, Compound 2 Form O, Compound 2 Form P), as well as compositions comprising novel salt forms of Compound 1 such as, for example, Compound 1 Fumarate (including amorphous Compound 1 Fumarate, Compound 1 Fumarate Form A, Compound 1 Fumarate Form B, Compound 1 Fumarate Form C), Compound 1 Adipate (including amorphous Compound 1 Adipate and Compound 1 Adipate Form A), and/or Compound 1 Succinate (including amorphous Compound 1 Succinate and Compound 1 Succinate Form A (i.e., Compound 2 Form A) and Compound 2 Form B, Compound 2 Form C, Compound 2 Form D, Compound 2 Form E, Compound 2 Form F, Compound 2 Form G, Compound 2 Form I, Compound 2 Form K, Compound 2 Form L, Compound 2 Form M, Compound 2 Form O, Compound 2 Form P).

### Compound 1 Fumarate Form A

A novel Compound 1 Fumarate Form A can be identified by X-ray Powder Diffraction (XRPD), having one or more characteristic diffractions at angles (2 theta±0.2) of 4.0, 5.3, 7.9, 10.2, 13.3, and 21.2. In some embodiments, Compound 1 Fumarate Form A can be identified by X-ray Powder Diffraction (XRPD), having one or more characteristic diffractions at angles (2 theta±0.2) of 4.0, 5.3, 7.9, 10.2, 13.3, and 21.2, corresponding to d-spacing (angstroms±0.2) of 22.1, 16.7, 11.1, 8.7, 6.7, and 4.2 (respectively).

A novel Compound 1 Fumarate Form A can be identified by X-ray Powder Diffraction (XRPD), having three or more characteristic diffractions at angles (2 theta±0.2) of 4.0, 5.3, 7.9, 10.2, 13.3, and 21.2. In some embodiments, Compound 1 Fumarate Form A can be identified by X-ray Powder Diffraction (XRPD), having three or more characteristic diffractions at angles (2 theta±0.2) of 4.0, 5.3, 7.9, 10.2, 13.3, and 21.2, corresponding to d-spacing (angstroms±0.2) of 22.1, 16.7, 11.1, 8.7, 6.7, and 4.2 (respectively).

In some embodiments, Compound 1 Fumarate Form A is characterized by an X-ray Power Diffraction having one or more peaks at substantially the same angles (2 theta±0.2) of:
4.0
5.3
6.6
7.9
10.2
13.3
14.8
17.2
18.6
21.2
24.1
26.5
28.4

In some embodiments, Compound 1 Fumarate Form A is characterized by an X-ray powder diffraction (XRPD) pattern having diffractions at angles (2 theta±0.2) and corresponding d-spacing (angstroms±0.2) of:

| 2 theta | d-spacing |
|---|---|
| 4.0 | 22.1 |
| 5.3 | 16.7 |
| 6.6 | 13.4 |
| 7.9 | 11.1 |
| 10.2 | 8.7 |
| 13.3 | 6.7 |
| 14.8 | 6.0 |
| 17.2 | 5.2 |
| 18.6 | 4.8 |
| 21.2 | 4.2 |
| 24.1 | 3.7 |
| 26.5 | 3.4 |
| 28.4 | 3.1 |

### Compound 1 Fumarate Form B

A novel Compound 1 Fumarate Form B can be identified by X-ray Powder Diffraction (XRPD), having one or more characteristic diffractions at angles (2 theta±0.2) of 5.4, 13.5, 16.3, 21.1, 23.5, 26.0, and 26.5. In some embodiments, Compound 1 Fumarate Form B can be identified by X-ray Powder Diffraction (XRPD), having one or more characteristic diffractions at angles (2 theta±0.2) of 5.4, 13.5, 16.3, 21.1, 23.5, 26.0, and 26.5, corresponding to d-spacing (angstroms±0.2) of 16.4, 6.6, 5.4, 4.2, 3.8, 3.4, and 3.4 (respectively).

A novel Compound 1 Fumarate Form B can be identified by X-ray Powder Diffraction (XRPD), having three or more characteristic diffractions at angles (2 theta±0.2) of 5.4, 13.5, 16.3, 21.1, 23.5, 26.0, and 26.5. In some embodiments, Compound 1 Fumarate Form B can be identified by X-ray Powder Diffraction (XRPD), having three or more characteristic diffractions at angles (2 theta±0.2) of 5.4, 13.5, 16.3, 21.1, 23.5, 26.0, and 26.5, corresponding to d-spacing (angstroms±0.2) of 16.4, 6.6, 5.4, 4.2, 3.8, 3.4, and 3.4 (respectively).

In some embodiments, Compound 1 Fumarate Form B is characterized by an X-ray Power Diffraction having one or more peaks at substantially the same angles (2 theta±0.2) of:
5.4
8.1
9.9
10.0
10.9
12.4
13.5
14.2
14.6
16.3
16.8
17.5
18.6
18.9
21.1
21.6
23.5
23.9
26.0
26.5
27.1
34.1
35.5
36.8

In some embodiments, Compound 1 Fumarate Form B is characterized by an X-ray powder diffraction (XRPD) pattern having diffractions at angles (2 theta±0.2) and corresponding d-spacing (angstroms±0.2) of:

| 2 theta | d-spacing |
|---|---|
| 5.4 | 16.4 |
| 8.1 | 10.9 |
| 9.9 | 9.0 |
| 10.0 | 8.8 |
| 10.9 | 8.1 |
| 12.4 | 7.2 |
| 13.5 | 6.6 |
| 14.2 | 6.2 |
| 14.6 | 6.1 |
| 16.3 | 5.4 |
| 16.8 | 5.3 |
| 17.5 | 5.1 |
| 18.6 | 4.8 |
| 18.9 | 4.7 |
| 21.1 | 4.2 |
| 21.6 | 4.1 |
| 23.5 | 3.8 |
| 23.9 | 3.7 |
| 26.0 | 3.4 |
| 26.5 | 3.4 |
| 27.1 | 3.3 |
| 34.1 | 2.6 |
| 35.5 | 2.5 |
| 36.8 | 2.4 |

### Compound 1 Fumarate Form C

A novel Compound 1 Fumarate Form C can be identified by X-ray Powder Diffraction (XRPD), having one or more characteristic diffractions at angles (2 theta±0.2) of 5.5, 8.2, 10.0, 18.1, 19.2, and 22.0. In some embodiments, Compound 1 Fumarate Form C can be identified by X-ray Powder Diffraction (XRPD), having one or more characteristic diffractions at angles (2 theta±0.2) of 5.5, 8.2, 10.0, 18.1, 19.2, and 22.0, corresponding to d-spacing (angstroms±0.2) of 16.1, 10.7, 8.9, 4.9, 4.6, and 4.0 (respectively).

A novel Compound 1 Fumarate Form C can be identified by X-ray Powder Diffraction (XRPD), having three or more characteristic diffractions at angles (2 theta±0.2) of 5.5, 8.2, 10.0, 18.1, 19.2, and 22.0. In some embodiments, Compound 1 Fumarate Form C can be identified by X-ray Powder Diffraction (XRPD), having three or more characteristic diffractions at angles (2 theta±0.2) of 5.5, 8.2, 10.0, 18.1, 19.2, and 22.0, corresponding to d-spacing (angstroms±0.2) of 16.1, 10.7, 8.9, 4.9, 4.6, and 4.0 (respectively).

In some embodiments, Compound 1 Fumarate Form C is characterized by an X-ray Power Diffraction having one or more peaks at substantially the same angles (2 theta±0.2) of:
5.5
8.2
10.0
13.7
14.6
18.1
19.2
22.0
24.1
25.3
27.4

In some embodiments, Compound 1 Fumarate Form C is characterized by an X-ray powder diffraction (XRPD) pattern having diffractions at angles (2 theta±0.2) and corresponding d-spacing (angstroms±0.2) of:

| 2 theta | d-spacing |
|---|---|
| 5.5 | 16.1 |
| 8.2 | 10.7 |
| 10.0 | 8.9 |
| 13.7 | 6.5 |
| 14.6 | 6.1 |
| 18.1 | 4.9 |
| 19.2 | 4.6 |
| 22.0 | 4.0 |
| 24.1 | 3.7 |
| 25.3 | 3.5 |
| 27.4 | 3.3 |

### Compound 1 Adipate Form A

A novel Compound 1 Adipate Form A can be identified by X-ray Powder Diffraction (XRPD), having one or more characteristic diffractions at angles (2 theta±0.2) of 7.9, 12.3, 15.7, 19.7, and 24.7. In some embodiments, Compound 1 Adipate Form A can be identified by X-ray Powder Diffraction (XRPD), having one or more characteristic diffractions at angles (2 theta±0.2) of 7.9, 12.3, 15.7, 19.7, and 24.7, corresponding to d-spacing (angstroms±0.2) of 11.2, 7.2, 5.6, 4.5, and 3.6 (respectively).

A novel Compound 1 Adipate Form A can be identified by X-ray Powder Diffraction (XRPD), having three or more characteristic diffractions at angles (2 theta±0.2) of 7.9, 12.3, 15.7, 19.7, and 24.7. In some embodiments, Compound 1 Adipate Form A can be identified by X-ray Powder Diffraction (XRPD), having three or more characteristic diffractions at angles (2 theta±0.2) of 7.9, 12.3, 15.7, 19.7, and 24.7, corresponding to d-spacing (angstroms±0.2) of 11.2, 7.2, 5.6, 4.5, and 3.6 (respectively).

In some embodiments, Compound 1 Adipate Form A is characterized by an X-ray Power Diffraction having one or more peaks at substantially the same angles (2 theta±0.2) of:
4.0
7.9
11.6
12.3
13.1
14.1
15.3
15.7
16.6
18.8
19.7
21.2
22.2
23.4
24.1
24.7
26.3
28.3
30.8

In some embodiments, Compound 1 Adipate Form A is characterized by an X-ray powder diffraction (XRPD) pattern having diffractions at angles (2 theta±0.2) and corresponding d-spacing (angstroms±0.2) of:

| 2 theta | d-spacing |
|---|---|
| 4.0 | 22.3 |
| 7.9 | 11.2 |
| 11.6 | 7.6 |
| 12.3 | 7.2 |
| 13.1 | 6.8 |
| 14.1 | 6.3 |
| 15.3 | 5.8 |
| 15.7 | 5.6 |
| 16.6 | 5.3 |
| 18.8 | 4.7 |
| 19.7 | 4.5 |
| 21.2 | 4.2 |
| 22.2 | 4.0 |
| 23.4 | 3.8 |
| 24.1 | 3.7 |
| 24.7 | 3.6 |
| 26.3 | 3.4 |
| 28.3 | 3.2 |
| 30.8 | 2.9 |

### Compound 1 Succinate Form A

A novel Compound 1 Succinate Form A (i.e., Compound 2 Form A) can be identified by X-ray Powder Diffraction (XRPD), having one or more characteristic diffractions at angles (2 theta±0.2) of 5.5, 8.3, 9.8, 17.9, 22.2, 24.8, 25.6, 36.1, and 37.3. In some embodiments, Compound 1 Succinate Form A (i.e., Compound 2 Form A) can be identified by X-ray Powder Diffraction (XRPD), having one or more characteristic diffractions at angles (2 theta±0.2) of 5.5, 8.3, 9.8, 17.9, 22.2, 24.8, 25.6, 36.1, and 37.3, corresponding to d-spacing (angstroms±0.2) of 16.0, 10.7, 9.0, 5.0, 4.0, 3.6, 3.5, 2.5, and 2.4 (respectively).

A novel Compound 1 Succinate Form A (i.e., Compound 2 Form A) can be identified by X-ray Powder Diffraction (XRPD), having three or more characteristic diffractions at angles (2 theta±0.2) of 5.5, 8.3, 9.8, 17.9, 22.2, 24.8, 25.6, 36.1, and 37.3. In some embodiments, Compound 1 Succinate Form A (i.e., Compound 2 Form A) can be identified by X-ray Powder Diffraction (XRPD), having three or more characteristic diffractions at angles (2 theta±0.2) of 5.5, 8.3, 9.8, 17.9, 22.2, 24.8, 25.6, 36.1, and 37.3, corresponding to d-spacing (angstroms±0.2) of 16.0, 10.7, 9.0, 5.0, 4.0, 3.6, 3.5, 2.5, and 2.4 (respectively).

In some embodiments, Compound 1 Succinate Form A (i.e., Compound 2 Form A) is characterized by an X-ray Power Diffraction having one or more peaks at substantially the same angles (2 theta±0.2) of:
5.5
8.3
9.8
10.8
12.5
12.6
13.8
14.4
14.6
16.7
17.1
17.9
18.4
18.7
19.0
19.6
20.0
20.5
20.9
21.3
21.8
22.2
22.5
23.1
23.4
24.2
24.8
25.3
25.6
26.5
26.8
27.3
28.1
28.5
29.1
29.7
30.7
32.3
32.9
33.8
34.9
36.1
37.3

In some embodiments, Compound 1 Succinate Form A (i.e., Compound 2 Form A) is characterized by an X-ray powder diffraction (XRPD) pattern having diffractions at angles (2 theta±0.2) and corresponding d-spacing (angstroms±0.2) of:

| 2 theta | d-spacing |
|---|---|
| 5.5 | 16.0 |
| 8.3 | 10.7 |
| 9.8 | 9.0 |
| 10.8 | 8.2 |
| 12.5 | 7.1 |
| 12.6 | 7.0 |
| 13.8 | 6.4 |
| 14.4 | 6.1 |
| 14.6 | 6.1 |
| 16.7 | 5.3 |
| 17.1 | 5.2 |
| 17.9 | 5.0 |
| 18.4 | 4.8 |
| 18.7 | 4.7 |
| 19.0 | 4.7 |
| 19.6 | 4.5 |
| 20.0 | 4.4 |
| 20.5 | 4.3 |
| 20.9 | 4.2 |
| 21.3 | 4.2 |
| 21.8 | 4.1 |
| 22.2 | 4.0 |
| 22.5 | 4.0 |
| 23.1 | 3.8 |
| 23.4 | 3.8 |
| 24.2 | 3.7 |
| 24.8 | 3.6 |
| 25.3 | 3.5 |
| 25.6 | 3.5 |
| 26.5 | 3.4 |
| 26.8 | 3.3 |
| 27.3 | 3.3 |
| 28.1 | 3.2 |
| 28.5 | 3.1 |
| 29.1 | 3.1 |
| 29.7 | 3.0 |
| 30.7 | 2.9 |
| 32.3 | 2.8 |
| 32.9 | 2.7 |
| 33.8 | 2.7 |
| 34.9 | 2.6 |
| 36.1 | 2.5 |
| 37.3 | 2.4 |

### Compound 1 Maleate Form A

A novel Compound 1 Maleate Form A can be identified by X-ray Powder Diffraction (XRPD), having one or more characteristic diffractions at angles (2 theta±0.2) of 3.1, 6.1, 9.2, 10.2, 17.7, and 19.3. In some embodiments, Compound 1 Maleate Form A can be identified by X-ray Powder Diffraction (XRPD), having one or more characteristic diffractions at angles (2 theta±0.2) of 3.1, 6.1, 9.2, 10.2, 17.7, and 19.3, corresponding to d-spacing (angstroms±0.2) of 28.6, 14.5, 9.6, 8.6, 5.0, and 4.6 (respectively).

A novel Compound 1 Maleate Form A can be identified by X-ray Powder Diffraction (XRPD), having three or more characteristic diffractions at angles (2 theta±0.2) of 3.1, 6.1, 9.2, 10.2, 17.7, and 19.3. In some embodiments, Compound 1 Maleate Form A can be identified by X-ray Powder Diffraction (XRPD), having three or more characteristic diffractions at angles (2 theta±0.2) of 3.1, 6.1, 9.2, 10.2, 17.7, and 19.3, corresponding to d-spacing (angstroms±0.2) of 28.6, 14.5, 9.6, 8.6, 5.0, and 4.6 (respectively).

In some embodiments, Compound 1 Maleate Form A is characterized by an X-ray Power Diffraction having one or more peaks at substantially the same angles (2 theta±0.2) of:
3.1
6.1
9.2
10.2
13.9
16.3
17.7
18.5
19.3
20.6
21.5
24.5

In some embodiments, Compound 1 Maleate Form A is characterized by an X-ray powder diffraction (XRPD) pattern having diffractions at angles (2 theta±0.2) and corresponding d-spacing (angstroms±0.2) of:

| 2 theta | d-spacing |
|---|---|
| 3.1 | 28.6 |
| 6.1 | 14.5 |
| 9.2 | 9.6 |
| 10.2 | 8.6 |
| 13.9 | 6.4 |
| 16.3 | 5.5 |
| 17.7 | 5.0 |
| 18.5 | 4.8 |
| 19.3 | 4.6 |
| 20.6 | 4.3 |
| 21.5 | 4.1 |
| 24.5 | 3.6 |

In some embodiments, the present disclosure provides a process for preparing a salt form of (S)-(5-cyclobutoxy-2-methyl-6-(1-(piperidin-4-yl)-1H-pyrazol-4-yl)-3,4-dihydroquinolin-1(2H)-yl)(cyclopropyl)methanone (“Compound 1”) comprising contacting Compound 1 with an acid selected from the group consisting of fumaric acid, adipic acid, and succinic acid, under conditions and for a time effective to form the corresponding salt form of Compound 1. In some embodiments, the present disclosure provides a process for preparing a salt form of Compound 1, comprising contacting Compound 1 with an acid selected from the group consisting of fumaric acid, adipic acid, succinic acid, and maleic acid, under conditions and for a time effective to form the corresponding salt form of Compound 1.

In some embodiments, the present disclosure provides a solid form of (S)-(5-cyclobutoxy-2-methyl-6-(1-(piperidin-4-yl)-1H-pyrazol-4-yl)-3,4-dihydroquinolin-1(2H)-yl)(cyclopropyl)methanone (“Compound 1”) obtained by a process comprising the step of contacting Compound 1 with succinic acid under conditions and for a time effective to form a succinate salt form of Compound 1. In some embodiments, a solid form of Compound 1 is obtained by a process comprising a step of contacting Compound 1 with fumaric acid under conditions and for a time effective to form a fumaric salt form of Compound 1. In some embodiments, a solid form of Compound 1 is obtained by a process comprising a step of contacting Compound 1 with adipic acid under conditions and for a time effective to form a adipic salt form of Compound 1. In some embodiments, a solid form of Compound 1 is obtained by a process comprising a step of contacting Compound 1 with maleic acid under conditions and for a time effective to form a maleic salt form of Compound 1.

In some embodiments, the present disclosure provides a composition comprising a crystalline salt form of Compound 1. In some embodiments, a composition comprises a crystalline salt form of Compound 1 and an amorphous salt form of Compound 1, wherein the amorphous salt form of Compound 1 is present in an amount selected from the following ranges: about 90 to about 99%, about 80 to about 89%, about 70 to about 79%, about 60 to about 69%, about 50 to about 59%, about 40 to about 49%, about 30 to about 39%, about 20 to about 29%, about 10 to about 19%, about 1 to about 9% and about 0 to about 0.99%. In some embodiments, a composition comprising a crystalline salt form of Compound 1 is substantially free of amorphous Compound 1.

In some embodiments, the present disclosure provides a composition comprising Compound 1 Fumarate Form A. In some embodiments, a composition comprising Compound 1 Fumarate Form A is substantially free of other crystalline forms of Compound 1. In some embodiments, a composition comprising Compound 1 Fumarate Form A is substantially free of an amorphous form of Compound 1.

In some embodiments, the present disclosure provides a composition comprising Compound 1 Fumarate Form B. In some embodiments, a composition comprising Compound 1 Fumarate Form B is substantially free of other crystalline forms of Compound 1. In some embodiments, a composition comprising Compound 1 Fumarate Form B is substantially free of an amorphous form of Compound 1.

In some embodiments, the present disclosure provides a composition comprising Compound 1 Fumarate Form C. In some embodiments, a composition comprising Compound 1 Fumarate Form C is substantially free of other crystalline forms of Compound 1. In some embodiments, a composition comprising Compound 1 Fumarate Form C is substantially free of an amorphous form of Compound 1.

In some embodiments, the present disclosure provides a composition comprising Compound 1 Adipate Form A. In some embodiments, a composition comprising Compound 1 Adipate Form A is substantially free of other crystalline forms of Compound 1. In some embodiments, a composition comprising Compound 1 Adipate Form A is substantially free of an amorphous form of Compound 1.

In some embodiments, the present disclosure provides a composition comprising Compound 1 Succinate Form A (i.e., Compound 2 Form A). In some embodiments, a composition comprising Compound 1 Succinate Form A (i.e., Compound 2 Form A) is substantially free of other crystalline forms of Compound 1. In some embodiments, a composition comprising Compound 1 Succinate Form A (i.e., Compound 2 Form A) is substantially free of an amorphous form of Compound 1.

In some embodiments, the present disclosure provides a composition comprising Compound 1 Maleate Form A. In some embodiments, a composition comprising Compound 1 Maleate Form A is substantially free of other crystalline forms of Compound 1. In some embodiments, a composition comprising Compound 1 Maleate Form A is substantially free of an amorphous form of Compound 1.

Pharmaceutical compositions reported herein can be combined with a pharmaceutically acceptable carrier or excipient. In some embodiments, pharmaceutical compositions reported herein can be provided in a unit dosage form container (e.g., in a vial or bag, or the like). In some embodiments, pharmaceutical compositions reported herein can be provided in an oral dosage form. In some embodiments, an oral dosage form is a capsule.

In some embodiments, the present disclosure provides a pharmaceutical composition for oral administration comprising a crystalline succinate salt of (S)-(5-cyclobutoxy-2-methyl-6-(1-(piperidin-4-yl)-1H-pyrazol-4-yl)-3,4-dihydroquinolin-1(2H)-yl)(cyclopropyl)methanone.

In some embodiments, the present disclosure provides a pharmaceutical composition for oral administration, comprising a succinate salt of (S)-(5-cyclobutoxy-2-methyl-6-(1-(piperidin-4-yl)-1H-pyrazol-4-yl)-3,4-dihydroquinolin-1(2H)-yl)(cyclopropyl)methanone.

In some embodiments, the present disclosure provides a pharmaceutical composition for oral administration, comprising a crystalline form of (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate.

In some embodiments, the present disclosure provides methods of inhibiting bromo and extra terminal (BET) bromodomains, comprising administering a salt form of Compound 1 to a subject. In some embodiments, the present disclosure provides methods of treating a disease, disorder, or condition responsive to inhibition of bromo and extra terminal (BET) bromodomains, comprising administering a salt form of Compound 1 to a subject in need thereof. In some embodiments, the disease, disorder, or condition is selected from cancer, inflammation, metabolic and neurological disorders, and infectious diseases.

### Solid Forms of Compound 2

As described above, a pharmaceutically acceptable salt of Compound 1 is a certain succinate salt, referred to as (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate (“Compound 2”):

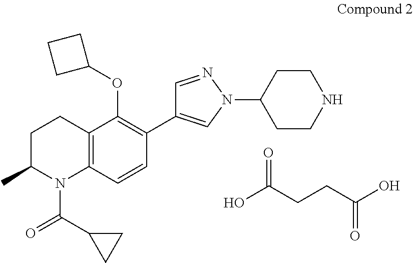

Compound 2 can occur in an amorphous solid form or in crystalline solid form or in mixtures of solid forms. Crystalline solid forms of Compound 2 can exist in one or more unique solid forms, which can additionally comprise one or more equivalents of water or solvent (i.e., hydrates or solvates, respectively). Accordingly, in some embodiments, the present disclosure provides a crystalline solid form of Compound 2.

As disclosed herein, crystalline forms of Compound 2 have distinct characteristic XRPD peaks (see Example 9) that are not reported in previous disclosures of Compound 1. Accordingly, provided herein are novel crystalline Compound 2 solid forms, pharmaceutical compositions thereof, and methods of preparing those crystalline Compound 2 solid forms and methods of use thereof.

Novel Compound 2 solid forms can be obtained by methods reported in Example 8 and Example 9. Different methods of preparation can lead to different solid forms. For example, Compound 2 Form A can be obtained from a solution comprising Compound 1 and succinic acid in acetone, as described in Example 3.

In some embodiments, certain solid forms of Compound 2 are converted from one solid form to another solid form. For example, subjecting Compound 2 Form A to certain conditions yields at least one of Compound 2 Form B, Form C, Form D, Form E, Form F, Form G, Form I, Form J, Form K, Form L, Form M, Form O, and/or Form P. The conditions suitable for converting Compound 2 Form A to any of Compound 2 Form B, Form C, Form D, Form E, Form F, Form G, Form I, Form J, Form K, Form L, Form M, Form O, and/or Form P include conditions such as anti-solvent addition; slow evaporation; crash cooling; slurrying at room temperature; slurrying at 50° C.; solid vapor diffusion; solution vapor diffusion; and grinding.

For example, certain solid forms of Compound 2 can be prepared by forming a suspension (i.e., “slurrying”) comprising Compound 2 Form A and a solvent, and maintaining the suspension for a period of time sufficient to generate certain solid forms of Compound 2 (e.g., Form B, Form C, Form D, Form E, Form F, Form G, Form I, Form J, Form K, Form L, Form M, Form O, and/or Form P). For example, exemplary solvents suitable to generate Form G include IPAc, MTBE, toluene, heptane, MIBK, EtOAc, ACN, acetone, H₂O/ACN, AcOH/n-heptane, MeOH/toluene, and CHCl₃/MTBE. In some embodiments, the suspension is maintained at room temperature. In some embodiments, the suspension is heated to a temperature between about 40° C. and about 80° C. In some embodiments, the suspension is heated to a temperature of about 50° C.

In some embodiments, the present disclosure provides a process for preparing Solid Form G of Compound 2, comprising suspending Compound 2 Form A in a solvent to provide a slurry and maintaining the slurry for a period of time sufficient to generate Compound 2 Form G. In some embodiments, the slurry is stirred. Exemplary solvents suitable to generate Form G include IPAc, MTBE, MIBK, and CHCl₃/MTBE. In some embodiments, the suspension is maintained at room temperature. In some embodiments, the suspension is heated to a temperature between about 40° C. and about 80° C. In some embodiments, the suspension is heated to a temperature of about 50° C.

In some embodiments, certain solid forms of Compound 2 can be prepared by anti-solvent addition to a solution of Compound 2 Form A. For example, certain solid forms of Compound 2 (e.g., Form B, Form C, Form D, Form E, Form F, Form G, Form I, Form J, Form K, Form L, Form M, Form O, and/or Form P) can be prepared by dissolving Compound 2 Form A in a solvent to obtain a solution, then adding an amount of anti-solvent sufficient to precipitate and/or provide a solid form of Compound 2. In some embodiments, the anti-solvent is miscible with the solvent. In some embodiments, Compound 2 is partially or completely insoluble in the anti-solvent. In some embodiments, the solvent is MeOH, EtOH, AcOH, CHCl₃, DCM, H₂O, DMSO and/or DMAc. In some embodiments, the anti-solvent is IPAc, MTBE, toluene, EtOAc, n-heptane, MIBK, ACN, and/or acetone.

In some embodiments, the present disclosure provides a process for preparing Solid Form G of Compound 2, comprising providing a solution comprising Compound 2 Form A and a solvent, and adding an amount of anti-solvent sufficient to precipitate a solid, wherein the solid is Solid Form G of Compound 2. In some embodiments, the solvent is DMAc. In some embodiments, the anti-solvent is MTBE. In some embodiments, a mixture of Compound 2 Form A, an anti-solvent, and a solvent is stored overnight to precipitate Solid Form G of Compound 2.

In some embodiments, certain solid forms of Compound 2 can be prepared by slow evaporation of Compound 2 Form A. For example, certain solid forms of Compound 2 (e.g., Form B, Form C, Form D, Form E, Form F, Form G, Form I, Form J, Form K, Form L, Form M, Form O, and/or Form P) can be prepared by dissolving Form A in a solvent to form a visually clear solution, then allowing the solvent to evaporate under ambient conditions to induce precipitation. Suitable examples of solvents include MeOH, EtOH, IPA, CHCl₃, DCM, H₂O, THF, MeOH/MTBE, EtOH/n-heptane, EtOH/MTBE, DCM/EtOAc, and/or CHCl₃/IPAc, CHCl₃/n-heptane.

In some embodiments, certain solid forms of Compound 2 can be prepared by crash cooling Compound 2 Form A. For example, certain solid forms of Compound 2 (e.g., Form B, Form C, Form D, Form E, Form F, Form G, Form I, Form J, Form K, Form L, Form M, Form O, and/or Form P) can be prepared by suspending Form A in a solvent to provide a slurry. The slurry is then heated to about 50° C., and then filtered over a membrane (e.g., a Nylon membrane having a pore size of about 0.45 μM). The filtrate is then cooled to about 5° C. and stored at conditions suitable to precipitate solids. Suitable examples of solvents include MeOH, EtOH, IPA, CHCl₃, DCM, H₂O, THF, MeOH/MTBE, EtOH/n-heptane, EtOH/MTBE, DCM/EtOAc, CHCl₃/IPAc, and/or CHCl₃/n-heptane.

In some embodiments, certain solid forms of Compound 2 can be prepared by solid vapor diffusion of Compound 2 Form A. For example, certain solid forms of Compound 2 (e.g., Form B, Form C, Form D, Form E, Form F, Form G, Form I, Form J, Form K, Form L, Form M, Form O, and/or Form P) can be prepared by contacting Compound 2 Form A with solvent vapors. Exemplary solvent vapors include H₂O, DCM, EtOH, MeOH, ACN, THF, CHCl₃, acetone, DMF, EtOAc, 1,4-dioxane, IPA, and/or DMSO.

In some embodiments, certain solid forms of Compound 2 can be prepared by solution vapor diffusion of Compound 2 Form A. For example, certain solid forms of Compound 2 (e.g., Form B, Form C, Form D, Form E, Form F, Form G, Form I, Form J, Form K, Form L, Form M, Form O, and/or Form P) can be prepared by dissolving Compound 2 Form A in a first solvent to provide a solution, and contacting the solution with solvent vapors of a second solvent. Suitable examples of a first solvent include MeOH, EtOH, AcOH, CHCl₃, DCM, DMSO, and DMAc. Suitable examples of solvent vapors include IPAc, MTBE, toluene, n-heptane, MIBK, and EtOAc.

As used herein, the term “precipitate” refers to the formation of a solid substance from a solution containing the same substance. A substance which precipitates from solution may be amorphous or crystalline. Precipitation may occur under a variety of conditions known to those of skill in the art, including the treatment of a solution of a solute (e.g., solute A in solvent B) with an antisolvent (i.e., a solvent that is miscible with solvent B, but does not significantly dissolve solute A). Non-limiting examples of solvent/antisolvent pairs include dimethylacetamide/methyl tert-butyl ether.

The solid forms of Compound 2 can be identified by various analytical techniques, such as X-ray powder diffraction (XRPD). The solid forms of Compound 2 disclosed herein include Compound 2 in Form A, Form B, Form C, Form D, Form E, Form F, Form G, Form I, Form K, Form L, Form M, Form O, and/or Form P, as well as compositions comprising a solid form of Compound 2 comprising one or more of Form A, Form B, Form C, Form D, Form E, Form F, Form G, Form I, Form K, Form L, Form M, Form O and/or Form P.

### Compound 2 Form A

The present disclosure provides a novel Compound 2 Form A (i.e., Compound 1 Succinate Form A). As described above, Compound 2 Form A (i.e., Compound 1 Succinate Form A) can be identified by X-ray Powder Diffraction (XRPD).

A novel Compound 2 Form A can be identified by X-ray Powder Diffraction (XRPD), having one or more characteristic diffractions at angles (2 theta±0.2) of 5.5, 8.3, 9.8, 17.9, 22.2, 24.8, 25.6, 36.1, and 37.3. In some embodiments, Compound 2 Form A can be identified by X-ray Powder Diffraction (XRPD), having one or more characteristic diffractions at angles (2 theta±0.2) of 5.5, 8.3, 9.8, 17.9, 22.2, 24.8, 25.6, 36.1, and 37.3, corresponding to d-spacing (angstroms±0.2) of 16.0, 10.7, 9.0, 5.0, 4.0, 3.6, 3.5, 2.5, and 2.4 (respectively).

A novel Compound 2 Form A can be identified by X-ray Powder Diffraction (XRPD), having three or more characteristic diffractions at angles (2 theta±0.2) of 5.5, 8.3, 9.8, 17.9, 22.2, 24.8, 25.6, 36.1, and 37.3. In some embodiments, Compound 2 Form A can be identified by X-ray Powder Diffraction (XRPD), having three or more characteristic diffractions at angles (2 theta±0.2) of 5.5, 8.3, 9.8, 17.9, 22.2, 24.8, 25.6, 36.1, and 37.3, corresponding to d-spacing (angstroms±0.2) of 16.0, 10.7, 9.0, 5.0, 4.0, 3.6, 3.5, 2.5, and 2.4 (respectively).

In some embodiments, Compound 2 Form A is characterized by an X-ray powder diffraction having one or more peaks at substantially the same angles (2 theta±0.2) of:
5.5
8.3
9.8
10.8
12.5
12.6
13.8
14.4
14.6
16.7
17.1
17.9
18.4
18.7
19.0
19.6
20.0
20.5
20.9
21.3
21.8
22.2
22.5
23.1
23.4
24.2
24.8
25.3
25.6
26.5
26.8
27.3
28.1
28.5
29.1
29.7
30.7
32.3
32.9
33.8
34.9
36.1
37.3

In some embodiments, Compound 2 Form A is characterized by an X-ray powder diffraction having one or more peaks at substantially the same angles (2 theta±0.2) and corresponding d-spacing (angstroms±0.2) of:

| 2 theta | d-spacing |
|---|---|
| 5.5 | 16.0 |
| 8.3 | 10.7 |
| 9.8 | 9.0 |
| 10.8 | 8.2 |
| 12.5 | 7.1 |
| 12.6 | 7.0 |
| 13.8 | 6.4 |
| 14.4 | 6.1 |
| 14.6 | 6.1 |
| 16.7 | 5.3 |
| 17.1 | 5.2 |
| 17.9 | 5.0 |
| 18.4 | 4.8 |
| 18.7 | 4.7 |
| 19.0 | 4.7 |
| 19.6 | 4.5 |
| 20.0 | 4.4 |
| 20.5 | 4.3 |
| 20.9 | 4.2 |
| 21.3 | 4.2 |
| 21.8 | 4.1 |
| 22.2 | 4.0 |
| 22.5 | 4.0 |
| 23.1 | 3.8 |
| 23.4 | 3.8 |
| 24.2 | 3.7 |
| 24.8 | 3.6 |
| 25.3 | 3.5 |
| 25.6 | 3.5 |
| 26.5 | 3.4 |
| 26.8 | 3.3 |
| 27.3 | 3.3 |
| 28.1 | 3.2 |
| 28.5 | 3.1 |
| 29.1 | 3.1 |
| 29.7 | 3.0 |
| 30.7 | 2.9 |
| 32.3 | 2.8 |
| 32.9 | 2.7 |
| 33.8 | 2.7 |
| 34.9 | 2.6 |
| 36.1 | 2.5 |
| 37.3 | 2.4 |

### Compound 2 Form B

A novel Compound 2 Form B can be identified by X-ray Powder Diffraction (XRPD), having one or more characteristic diffractions at angles (2 theta±0.2) of 4.7, 5.9, 8.7, 11.0, 17.2, and 20.4. In some embodiments, Compound 2 Form B can be identified by X-ray Powder Diffraction (XRPD), having one or more characteristic diffractions at angles (2 theta±0.2) of 4.7, 5.9, 8.7, 11.0, 17.2, and 20.4, corresponding to d-spacing (angstroms±0.2) of 18.7, 15.1, 10.1, 8.1, 5.1, and 4.4 (respectively).

A novel Compound 2 Form B can be identified by X-ray Powder Diffraction (XRPD), having three or more characteristic diffractions at angles (2 theta±0.2) of 4.7, 5.9, 8.7, 11.0, 17.2, and 20.4. In some embodiments, Compound 2 Form B can be identified by X-ray Powder Diffraction (XRPD), having three or more characteristic diffractions at angles (2 theta±0.2) of 4.7, 5.9, 8.7, 11.0, 17.2, and 20.4, corresponding to d-spacing (angstroms±0.2) of 18.7, 15.1, 10.1, 8.1, 5.1, and 4.4 (respectively).

In some embodiments, Compound 2 Form B is characterized by an X-ray powder diffraction having one or more peaks at substantially the same angles (2 theta±0.2) of:
4.7
5.9
8.7
9.7
10.3
11.0
11.7
13.8
14.5
16.2
17.2
17.7
18.5
18.8
19.1
20.4
20.6
21.2
21.6
22.6
23.8
24.3
24.9
25.2
26.1
26.9
27.7
28.5
30.0
32.7
33.6
35.1

In some embodiments, Compound 2 Form B is characterized by an X-ray powder diffraction having one or more peaks at substantially the same angles (2 theta±0.2) and corresponding d-spacing (angstroms±0.2) of:

| 2 theta | d-spacing |
|---|---|
| 4.7 | 18.7 |
| 5.9 | 15.1 |
| 8.7 | 10.1 |
| 9.7 | 9.1 |
| 10.3 | 8.6 |
| 11.0 | 8.1 |
| 11.7 | 7.5 |
| 13.8 | 6.4 |
| 14.5 | 6.1 |
| 16.2 | 5.5 |
| 17.2 | 5.1 |
| 17.7 | 5.0 |
| 18.5 | 4.8 |
| 18.8 | 4.7 |
| 19.1 | 4.6 |
| 20.4 | 4.4 |
| 20.6 | 4.3 |
| 21.2 | 4.2 |
| 21.6 | 4.1 |
| 22.6 | 3.9 |
| 23.8 | 3.7 |
| 24.3 | 3.7 |
| 24.9 | 3.6 |
| 25.2 | 3.5 |
| 26.1 | 3.4 |
| 26.9 | 3.3 |
| 27.7 | 3.2 |
| 28.5 | 3.1 |
| 30.0 | 3.0 |
| 32.7 | 2.7 |
| 33.6 | 2.7 |
| 35.1 | 2.6 |

### Compound 2 Form C

A novel Compound 2 Form C can be identified by X-ray Powder Diffraction (XRPD), having one or more characteristic diffractions at angles (2 theta±0.2) of 5.4, 8.1, 10.1, 10.9, 16.4, 17.7, and 34.3. In some embodiments, Compound 2 Form C can be identified by X-ray Powder Diffraction (XRPD), having one or more characteristic diffractions at angles (2 theta±0.2) of 5.4, 8.1, 10.1, 10.9, 16.4, 17.7, and 34.3, corresponding to d-spacing (angstroms±0.2) of 16.3, 10.9, 8.7, 8.1, 5.4, 5.0, and 2.6 (respectively).

A novel Compound 2 Form C can be identified by X-ray Powder Diffraction (XRPD), having three or more characteristic diffractions at angles (2 theta±0.2) of 5.4, 8.1, 10.1, 10.9, 16.4, 17.7, and 34.3. In some embodiments, Compound 2 Form C can be identified by X-ray Powder Diffraction (XRPD), having three or more characteristic diffractions at angles (2 theta±0.2) of 5.4, 8.1, 10.1, 10.9, 16.4, 17.7, and 34.3, corresponding to d-spacing (angstroms±0.2) of 16.3, 10.9, 8.7, 8.1, 5.4, 5.0, and 2.6 (respectively).

In some embodiments, Compound 2 Form C is characterized by an X-ray powder diffraction having one or more peaks at substantially the same angles (2 theta±0.2) of:
5.4
8.1
9.9
10.1
10.9
12.4
14.2
14.8
16.4
17.1
17.7
18.6
19.3
19.9
20.7
21.2
21.8
22.6
23.2
24.2
24.6
25.2
26.0
27.3
29.3
30.0
34.3
37.5

In some embodiments, Compound 2 Form C is characterized by an X-ray powder diffraction having one or more peaks at substantially the same angles (2 theta±0.2) and corresponding d-spacing (angstroms±0.2) of:

| 2 theta | d-spacing |
|---|---|
| 5.4 | 16.3 |
| 8.1 | 10.9 |
| 9.9 | 8.9 |
| 10.1 | 8.7 |
| 10.9 | 8.1 |
| 12.4 | 7.1 |
| 14.2 | 6.2 |
| 14.8 | 6.0 |
| 16.4 | 5.4 |
| 17.1 | 5.2 |
| 17.7 | 5.0 |
| 18.6 | 4.8 |
| 19.3 | 4.6 |
| 19.9 | 4.5 |
| 20.7 | 4.3 |
| 21.2 | 4.2 |
| 21.8 | 4.1 |
| 22.6 | 3.9 |
| 23.2 | 3.8 |
| 24.2 | 3.7 |
| 24.6 | 3.6 |
| 25.2 | 3.5 |
| 26.0 | 3.4 |
| 27.3 | 3.3 |
| 29.3 | 3.0 |
| 30.0 | 3.0 |
| 34.3 | 2.6 |
| 37.5 | 2.4 |

### Compound 2 Form D

A novel Compound 2 Form D can be identified by X-ray Powder Diffraction (XRPD), having one or more characteristic diffractions at angles (2 theta±0.2) of 5.4, 14.7, 18.5, 21.8, and 38.6. In some embodiments, Compound 2 Form D can be identified by X-ray Powder Diffraction (XRPD), having one or more characteristic diffractions at angles (2 theta±0.2) of 5.4, 14.7, 18.5, 21.8, and 38.6, corresponding to d-spacing (angstroms±0.2) of 16.2, 6.0, 4.8, 4.1, and 2.3 (respectively).

A novel Compound 2 Form D can be identified by X-ray Powder Diffraction (XRPD), having three or more characteristic diffractions at angles (2 theta±0.2) of 5.4, 14.7, 18.5, 21.8, and 38.6. In some embodiments, Compound 2 Form D can be identified by X-ray Powder Diffraction (XRPD), having three or more characteristic diffractions at angles (2 theta±0.2) of 5.4, 14.7, 18.5, 21.8, and 38.6, corresponding to d-spacing (angstroms±0.2) of 16.2, 6.0, 4.8, 4.1, and 2.3 (respectively).

In some embodiments, Compound 2 Form D is characterized by an X-ray powder diffraction having one or more peaks at substantially the same angles (2 theta±0.2) of:
5.4
8.1
9.8
10.0
10.8
12.4
13.6
14.2
14.7
16.8
17.3
18.5
19.1
19.9
20.5
20.9
21.8
23.5
24.1
25.1
25.7
26.1
27.6
29.6
33.4
35.8
38.6

In some embodiments, Compound 2 Form D is characterized by an X-ray powder diffraction having one or more peaks at substantially the same angles (2 theta±0.2) and corresponding d-spacing (angstroms±0.2) of:

| 2 theta | d-spacing |
|---|---|
| 5.4 | 16.2 |
| 8.1 | 10.9 |
| 9.8 | 9.0 |
| 10.0 | 8.9 |
| 10.8 | 8.2 |
| 12.4 | 7.2 |
| 13.6 | 6.5 |
| 14.2 | 6.2 |
| 14.7 | 6.0 |
| 16.8 | 5.3 |
| 17.3 | 5.1 |
| 18.5 | 4.8 |
| 19.1 | 4.7 |
| 19.9 | 4.5 |
| 20.5 | 4.3 |
| 20.9 | 4.2 |
| 21.8 | 4.1 |
| 23.5 | 3.8 |
| 24.1 | 3.7 |
| 25.1 | 3.5 |
| 25.7 | 3.5 |
| 26.1 | 3.4 |
| 27.6 | 3.2 |
| 29.6 | 3.0 |
| 33.4 | 2.7 |
| 35.8 | 2.5 |
| 38.6 | 2.3 |

### Compound 2 Form E

A novel Compound 2 Form E can be identified by X-ray Powder Diffraction (XRPD), having one or more characteristic diffractions at angles (2 theta±0.2) of 5.5, 11.3, 13.0, 16.3, and 20.3. In some embodiments, Compound 2 Form E can be identified by X-ray Powder Diffraction (XRPD), having one or more characteristic diffractions at angles (2 theta±0.2) of 5.5, 11.3, 13.0, 16.3, and 20.3, corresponding to d-spacing (angstroms±0.2) of 16.0, 7.8, 6.8, 5.4, and 4.4 (respectively).

A novel Compound 2 Form E can be identified by X-ray Powder Diffraction (XRPD), having three or more characteristic diffractions at angles (2 theta±0.2) of 5.5, 11.3, 13.0, 16.3, and 20.3. In some embodiments, Compound 2 Form E can be identified by X-ray Powder Diffraction (XRPD), having three or more characteristic diffractions at angles (2 theta±0.2) of 5.5, 11.3, 13.0, 16.3, and 20.3, corresponding to d-spacing (angstroms±0.2) of 16.0, 7.8, 6.8, 5.4, and 4.4 (respectively).

In some embodiments, Compound 2 Form E is characterized by an X-ray powder diffraction having one or more peaks at substantially the same angles (2 theta±0.2) of:
5.5
8.3
9.8
10.1
10.7
11.3
12.2
13.0
13.8
14.1
15.0
16.3
16.5
17.3
18.2
18.6
19.0
19.6
20.3
20.9
21.2
22.1
22.9
23.6
24.2
26.2
27.2
28.1
30.4
32.8
35.3
36.3
37.0

In some embodiments, Compound 2 Form E is characterized by an X-ray powder diffraction having one or more peaks at substantially the same angles (2 theta±0.2) and corresponding d-spacing (angstroms±0.2) of:

| 2 theta | d-spacing |
|---|---|
| 5.5 | 16.0 |
| 8.3 | 10.7 |
| 9.8 | 9.1 |
| 10.1 | 8.8 |
| 10.7 | 8.3 |
| 11.3 | 7.8 |
| 12.2 | 7.2 |
| 13.0 | 6.8 |
| 13.8 | 6.4 |
| 14.1 | 6.3 |
| 15.0 | 5.9 |
| 16.3 | 5.4 |
| 16.5 | 5.4 |
| 17.3 | 5.1 |
| 18.2 | 4.9 |
| 18.6 | 4.8 |
| 19.0 | 4.7 |
| 19.6 | 4.5 |
| 20.3 | 4.4 |
| 20.9 | 4.3 |
| 21.2 | 4.2 |
| 22.1 | 4.0 |
| 22.9 | 3.9 |
| 23.6 | 3.8 |
| 24.2 | 3.7 |
| 26.2 | 3.4 |
| 27.2 | 3.3 |
| 28.1 | 3.2 |
| 30.4 | 2.9 |
| 32.8 | 2.7 |
| 35.3 | 2.5 |
| 36.3 | 2.5 |
| 37.0 | 2.4 |

### Compound 2 Form F

A novel Compound 2 Form F can be identified by X-ray Powder Diffraction (XRPD), having one or more characteristic diffractions at angles (2 theta±0.2) of 13.6, 14.2, 16.3, 21.8, and 26.7. In some embodiments, Compound 2 Form F can be identified by X-ray Powder Diffraction (XRPD), having one or more characteristic diffractions at angles (2 theta±0.2) of 13.6, 14.2, 16.3, 21.8, and 26.7, corresponding to d-spacing (angstroms±0.2) of 6.5, 6.2, 5.4, 4.1, and 3.3 (respectively).

A novel Compound 2 Form F can be identified by X-ray Powder Diffraction (XRPD), having three or more characteristic diffractions at angles (2 theta±0.2) of 13.6, 14.2, 16.3, 21.8, and 26.7. In some embodiments, Compound 2 Form F can be identified by X-ray Powder Diffraction (XRPD), having three or more characteristic diffractions at angles (2 theta±0.2) of 13.6, 14.2, 16.3, 21.8, and 26.7, corresponding to d-spacing (angstroms±0.2) of 6.5, 6.2, 5.4, 4.1, and 3.3 (respectively).

In some embodiments, Compound 2 Form F is characterized by an X-ray powder diffraction having one or more peaks at substantially the same angles (2 theta±0.2) of:
5.5
8.2
9.7
9.9
10.7
12.3
13.6
14.2
14.6
16.3
16.8
18.6
18.9
19.1
19.5
20.3
20.8
21.0
21.8
22.9
23.5
23.8
24.1
26.1
26.7
27.4
29.4

In some embodiments, Compound 2 Form F is characterized by an X-ray powder diffraction having one or more peaks at substantially the same angles (2 theta±0.2) and corresponding d-spacing (angstroms±0.2) of:

| 2 theta | d-spacing |
|---|---|
| 5.5 | 16.1 |
| 8.2 | 10.8 |
| 9.7 | 9.1 |
| 9.9 | 8.9 |
| 10.7 | 8.3 |
| 12.3 | 7.2 |
| 13.6 | 6.5 |
| 14.2 | 6.2 |
| 14.6 | 6.0 |
| 16.3 | 5.4 |
| 16.8 | 5.3 |
| 18.6 | 4.8 |
| 18.9 | 4.7 |
| 19.1 | 4.6 |
| 19.5 | 4.6 |
| 20.3 | 4.4 |
| 20.8 | 4.3 |
| 21.0 | 4.2 |
| 21.8 | 4.1 |
| 22.9 | 3.9 |
| 23.5 | 3.8 |
| 23.8 | 3.7 |
| 24.1 | 3.7 |
| 26.1 | 3.4 |
| 26.7 | 3.3 |
| 27.4 | 3.3 |
| 29.4 | 3.0 |

### Compound 2 Form G

A novel Compound 2 Form G can be identified by X-ray Powder Diffraction (XRPD), having one or more characteristic diffractions at angles (2 theta±0.2) of 4.4, 6.8, 9.1, 15.3, and 38.8. In some embodiments, Solid Form G of Compound 2 can be identified by a XRPD pattern having one or more characteristic diffractions at angles (2 theta±0.2) 4.4, 6.8, 9.1, 13.1, 15.3, 16.1, 19.6, 36.6, and 38.8. In some embodiments, Solid Form G of Compound 2 can be identified by X-ray Powder Diffraction (XRPD), having one or more characteristic diffractions at angles (2 theta±0.2) of 4.4, 6.8, 9.1, 13.1, 15.3, 16.1, 19.6, 36.6, and 38.8, corresponding to d-spacing (angstroms±0.2) of 20.1, 13.0, 9.7, 6.8, 5.8, 5.5, 4.5, 2.5, and 2.3 (respectively).

A novel Compound 2 Form G can be identified by X-ray Powder Diffraction (XRPD), having three or more characteristic diffractions at angles (2 theta±0.2) of 4.4, 6.8, 9.1, 15.3, and 38.8. In some embodiments, Solid Form G of Compound 2 can be identified by a XRPD pattern having three or more characteristic diffractions at angles (2 theta±0.2) 4.4, 6.8, 9.1, 13.1, 15.3, 16.1, 19.6, 36.6, and 38.8. In some embodiments, Solid Form G of Compound 2 can be identified by X-ray Powder Diffraction (XRPD), having three or more characteristic diffractions at angles (2 theta±0.2) of 4.4, 6.8, 9.1, 13.1, 15.3, 16.1, 19.6, 36.6, and 38.8, corresponding to d-spacing (angstroms±0.2) of 20.1, 13.0, 9.7, 6.8, 5.8, 5.5, 4.5, 2.5, and 2.3 (respectively).

Solid Form G Compound 2 can be characterized by an X-ray powder diffraction having one or more peaks at substantially the same angles (2 theta±0.2) of:
4.4
6.8
7.7
8.7
9.1
10.5
11.1
11.8
13.1
13.9
14.9
15.3
16.1
16.6
16.9
17.4
18.0
18.2
18.9
19.2
19.6
20.0
20.7
21.1
21.6
21.8
22.1
22.5
23.0
23.5
24.5
25.3
25.8
26.2
26.8
27.5
27.9
28.4
28.7
29.1
29.9
31.0
32.7
33.3
34.0
35.2
36.1
36.6
38.8

In some embodiments, Compound 2 Form G is characterized by an X-ray powder diffraction having one or more peaks at substantially the same angles (2 theta±0.2) and corresponding d-spacing (angstroms±0.2) of:

| 2 theta | d-spacing |
|---|---|
| 4.4 | 20.1 |
| 6.8 | 13.0 |
| 7.7 | 11.4 |
| 8.7 | 10.1 |
| 9.1 | 9.7 |
| 10.5 | 8.4 |
| 11.1 | 7.9 |
| 11.8 | 7.5 |
| 13.1 | 6.8 |
| 13.9 | 6.4 |
| 14.9 | 5.9 |
| 15.3 | 5.8 |
| 16.1 | 5.5 |
| 16.6 | 5.3 |
| 16.9 | 5.2 |
| 17.4 | 5.1 |
| 18.0 | 4.9 |
| 18.2 | 4.9 |
| 18.9 | 4.7 |
| 19.2 | 4.6 |
| 19.6 | 4.5 |
| 20.0 | 4.4 |
| 20.7 | 4.3 |
| 21.1 | 4.2 |
| 21.6 | 4.1 |
| 21.8 | 4.1 |
| 22.1 | 4.0 |
| 22.5 | 4.0 |
| 23.0 | 3.9 |
| 23.5 | 3.8 |
| 24.5 | 3.6 |
| 25.3 | 3.5 |
| 25.8 | 3.5 |
| 26.2 | 3.4 |
| 26.8 | 3.3 |
| 27.5 | 3.2 |
| 27.9 | 3.2 |
| 28.4 | 3.1 |
| 28.7 | 3.1 |
| 29.1 | 3.1 |
| 29.9 | 3.0 |
| 31.0 | 2.9 |
| 32.7 | 2.7 |
| 33.3 | 2.7 |
| 34.0 | 2.6 |
| 35.2 | 2.6 |
| 36.1 | 2.5 |
| 36.6 | 2.5 |
| 38.8 | 2.3 |

### Compound 2 Form I

A novel Compound 2 Form I can be identified by X-ray Powder Diffraction (XRPD), having one or more characteristic diffractions at angles (2 theta±0.2) of 3.4, 5.9, 9.2, 10.3, 11.0, and 25.4. In some embodiments, Compound 2 Form I can be identified by X-ray Powder Diffraction (XRPD), having one or more characteristic diffractions at angles (2 theta±0.2) of 3.4, 5.9, 9.2, 10.3, 11.0, and 25.4, corresponding to d-spacing (angstroms±0.2) of 25.8, 15.0, 9.7, 8.6, 8.0, and 3.5 (respectively).

A novel Compound 2 Form I can be identified by X-ray Powder Diffraction (XRPD), having three or more characteristic diffractions at angles (2 theta±0.2) of 3.4, 5.9, 9.2, 10.3, 11.0, and 25.4. In some embodiments, Compound 2 Form I can be identified by X-ray Powder Diffraction (XRPD), having three or more characteristic diffractions at angles (2 theta±0.2) of 3.4, 5.9, 9.2, 10.3, 11.0, and 25.4, corresponding to d-spacing (angstroms±0.2) of 25.8, 15.0, 9.7, 8.6, 8.0, and 3.5 (respectively).

In some embodiments, Compound 2 Form I is characterized by an X-ray powder diffraction having one or more peaks at substantially the same angles (2 theta±0.2) of:
3.4
5.9
6.7
8.0
8.8
9.2
9.5
9.8
10.1
10.3
10.8
11.0
12.5
12.9
13.2
13.9
14.5
15.7
16.8
17.4
18.3
18.9
19.8
20.7
21.4
23.6
25.4
27.0
28.3
29.1
29.9

In some embodiments, Compound 2 Form I is characterized by an X-ray powder diffraction having one or more peaks at substantially the same angles (2 theta±0.2) and corresponding d-spacing (angstroms±0.2) of:

| 2 theta | d-spacing |
|---|---|
| 3.4 | 25.8 |
| 5.9 | 15.0 |
| 6.7 | 13.1 |
| 8.0 | 11.1 |
| 8.8 | 10.0 |
| 9.2 | 9.7 |
| 9.5 | 9.4 |
| 9.8 | 9.0 |
| 10.1 | 8.8 |
| 10.3 | 8.6 |
| 10.8 | 8.2 |
| 11.0 | 8.0 |
| 12.5 | 7.1 |
| 12.9 | 6.9 |
| 13.2 | 6.7 |
| 13.9 | 6.4 |
| 14.5 | 6.1 |
| 15.7 | 5.6 |
| 16.8 | 5.3 |
| 17.4 | 5.1 |
| 18.3 | 4.8 |
| 18.9 | 4.7 |
| 19.8 | 4.5 |
| 20.7 | 4.3 |
| 21.4 | 4.2 |
| 23.6 | 3.8 |
| 25.4 | 3.5 |
| 27.0 | 3.3 |
| 28.3 | 3.2 |
| 29.1 | 3.1 |
| 29.9 | 3.0 |

### Compound 2 Form J

A novel Compound 2 Form J can be identified by X-ray Powder Diffraction (XRPD), having one or more characteristic diffractions at angles (2 theta±0.2) of 5.4, 8.1, 12.4, 13.5, 15.6, 21.0, and 21.7. In some embodiments, Compound 2 Form J can be identified by X-ray Powder Diffraction (XRPD), having one or more characteristic diffractions at angles (2 theta±0.2) of 5.4, 8.1, 12.4, 13.5, 15.6, 21.0, and 21.7, corresponding to d-spacing (angstroms±0.2) of 16.3, 10.9, 7.1, 6.5, 5.7, 4.2, and 4.1 (respectively).

A novel Compound 2 Form J can be identified by X-ray Powder Diffraction (XRPD), having three or more characteristic diffractions at angles (2 theta±0.2) of 5.4, 8.1, 12.4, 13.5, 15.6, 21.0, and 21.7. In some embodiments, Compound 2 Form J can be identified by X-ray Powder Diffraction (XRPD), having three or more characteristic diffractions at angles (2 theta 0.2) of 5.4, 8.1, 12.4, 13.5, 15.6, 21.0, and 21.7, corresponding to d-spacing (angstroms±0.2) of 16.3, 10.9, 7.1, 6.5, 5.7, 4.2, and 4.1 (respectively).

In some embodiments, Compound 2 Form J is characterized by an X-ray powder diffraction having one or more peaks at substantially the same angles (2 theta±0.2) of:
5.4
8.1
9.8
10.0
10.8
12.4
13.5
15.6
18.7
19.0
21.0
21.7
23.5
35.6
38.5

In some embodiments, Compound 2 Form J is characterized by an X-ray powder diffraction having one or more peaks at substantially the same angles (2 theta±0.2) and corresponding d-spacing (angstroms±0.2) of:

| 2 theta | d-spacing |
|---|---|
| 5.4 | 16.3 |
| 8.1 | 10.9 |
| 9.8 | 9.0 |
| 10.0 | 8.8 |
| 10.8 | 8.2 |
| 12.4 | 7.1 |
| 13.5 | 6.5 |
| 15.6 | 5.7 |
| 18.7 | 4.7 |
| 19.0 | 4.7 |
| 21.0 | 4.2 |
| 21.7 | 4.1 |
| 23.5 | 3.8 |
| 35.6 | 2.5 |
| 38.5 | 2.3 |

### Compound 2 Form K

A novel Compound 2 Form K can be identified by X-ray Powder Diffraction (XRPD), having one or more characteristic diffractions at angles (2 theta±0.2) of 5.5, 8.7, 9.7, 22.2, and 24.4. In some embodiments, Compound 2 Form K can be identified by X-ray Powder Diffraction (XRPD), having one or more characteristic diffractions at angles (2 theta±0.2) of 5.5, 8.7, 9.7, 22.2, and 24.4, corresponding to d-spacing (angstroms±0.2) of 15.9, 10.2, 9.1, 4.0, and 3.7 (respectively).

A novel Compound 2 Form K can be identified by X-ray Powder Diffraction (XRPD), having three or more characteristic diffractions at angles (2 theta±0.2) of 5.5, 8.7, 9.7, 22.2, and 24.4. In some embodiments, Compound 2 Form K can be identified by X-ray Powder Diffraction (XRPD), having three or more characteristic diffractions at angles (2 theta±0.2) of 5.5, 8.7, 9.7, 22.2, and 24.4, corresponding to d-spacing (angstroms±0.2) of 15.9, 10.2, 9.1, 4.0, and 3.7 (respectively).

In some embodiments, Compound 2 Form K is characterized by an X-ray powder diffraction having one or more peaks at substantially the same angles (2 theta±0.2) of:
5.5
8.3
8.7
9.7
10.9
14.5
16.8
17.6
18.7
20.5
22.2
24.4

In some embodiments, Compound 2 Form K is characterized by an X-ray powder diffraction having one or more peaks at substantially the same angles (2 theta±0.2) and corresponding d-spacing (angstroms±0.2) of:

| 2 theta | d-spacing |
|---|---|
| 5.5 | 15.9 |
| 8.3 | 10.7 |
| 8.7 | 10.2 |
| 9.7 | 9.1 |
| 10.9 | 8.1 |
| 14.5 | 6.1 |
| 16.8 | 5.3 |
| 17.6 | 5.0 |
| 18.7 | 4.7 |
| 20.5 | 4.3 |
| 22.2 | 4.0 |
| 24.4 | 3.7 |

### Compound 2 Form L

A novel Compound 2 Form L can be identified by X-ray Powder Diffraction (XRPD), having one or more characteristic diffractions at angles (2 theta±0.2) of 5.5, 9.8, 12.6, 17.8, 18.9, 21.0, 22.2, and 29.2. In some embodiments, Compound 2 Form L can be identified by X-ray Powder Diffraction (XRPD), having one or more characteristic diffractions at angles (2 theta±0.2) of 5.5, 9.8, 12.6, 17.8, 18.9, 21.0, 22.2, and 29.2, corresponding to d-spacing (angstroms 0.2) of 16.0, 9.0, 7.0, 5.0, 4.7, 4.2, 4.0, and 3.1 (respectively).

A novel Compound 2 Form L can be identified by X-ray Powder Diffraction (XRPD), having three or more characteristic diffractions at angles (2 theta±0.2) of 5.5, 9.8, 12.6, 17.8, 18.9, 21.0, 22.2, and 29.2. In some embodiments, Compound 2 Form L can be identified by X-ray Powder Diffraction (XRPD), having three or more characteristic diffractions at angles (2 theta±0.2) of 5.5, 9.8, 12.6, 17.8, 18.9, 21.0, 22.2, and 29.2, corresponding to d-spacing (angstroms±0.2) of 16.0, 9.0, 7.0, 5.0, 4.7, 4.2, 4.0, and 3.1 (respectively).

In some embodiments, Compound 2 Form L is characterized by an X-ray powder diffraction having one or more peaks at substantially the same angles (2 theta±0.2) of:
5.5
8.3
9.8
11.0
12.6
13.9
14.6
16.6
17.0
17.8
18.7
18.9
19.5
19.8
21.0
22.2
22.8
23.7
24.1
25.0
25.8
26.5
26.8
28.2
29.2

In some embodiments, Compound 2 Form L is characterized by an X-ray powder diffraction having one or more peaks at substantially the same angles (2 theta±0.2) and corresponding d-spacing (angstroms±0.2) of:

| 2 theta | d-spacing |
|---|---|
| 5.5 | 16.0 |
| 8.3 | 10.6 |
| 9.8 | 9.0 |
| 11.0 | 8.1 |
| 12.6 | 7.0 |
| 13.9 | 6.4 |
| 14.6 | 6.1 |
| 16.6 | 5.3 |
| 17.0 | 5.2 |
| 17.8 | 5.0 |
| 18.7 | 4.7 |
| 18.9 | 4.7 |
| 19.5 | 4.6 |
| 19.8 | 4.5 |
| 21.0 | 4.2 |
| 22.2 | 4.0 |
| 22.8 | 3.9 |
| 23.7 | 3.8 |
| 24.1 | 3.7 |
| 25.0 | 3.6 |
| 25.8 | 3.5 |
| 26.5 | 3.4 |
| 26.8 | 3.3 |
| 28.2 | 3.2 |
| 29.2 | 3.1 |

### Compound 2 Form M

A novel Compound 2 Form M can be identified by X-ray Powder Diffraction (XRPD), having one or more characteristic diffractions at angles (2 theta±0.2) of 5.5, 8.3, 14.5, 19.4, 22.1, 24.9, and 37.7. In some embodiments, Compound 2 Form M can be identified by X-ray Powder Diffraction (XRPD), having one or more characteristic diffractions at angles (2 theta±0.2) of 5.5, 8.3, 14.5, 19.4, 22.1, 24.9, and 37.7, corresponding to d-spacing (angstroms±0.2) of 16.0, 10.7, 6.1, 4.6, 4.0, 3.6, and 2.4 (respectively).

A novel Compound 2 Form M can be identified by X-ray Powder Diffraction (XRPD), having three or more characteristic diffractions at angles (2 theta±0.2) of 5.5, 8.3, 14.5, 19.4, 22.1, 24.9, and 37.7. In some embodiments, Compound 2 Form M can be identified by X-ray Powder Diffraction (XRPD), having three or more characteristic diffractions at angles (2 theta±0.2) of 5.5, 8.3, 14.5, 19.4, 22.1, 24.9, and 37.7, corresponding to d-spacing (angstroms±0.2) of 16.0, 10.7, 6.1, 4.6, 4.0, 3.6, and 2.4 (respectively).

In some embodiments, Compound 2 Form M is characterized by an X-ray powder diffraction having one or more peaks at substantially the same angles (2 theta±0.2) of:
5.5
8.3
9.8
10.8
12.4
13.8
14.5
16.7
17.8
19.0
19.4
21.5
22.1
24.0
24.2
24.9
26.7
36.4
37.7

In some embodiments, Compound 2 Form M is characterized by an X-ray powder diffraction having one or more peaks at substantially the same angles (2 theta±0.2) and corresponding d-spacing (angstroms±0.2) of:

| 2 theta | d-spacing |
|---|---|
| 5.5 | 16.0 |
| 8.3 | 10.7 |
| 9.8 | 9.0 |
| 10.8 | 8.2 |
| 12.4 | 7.1 |
| 13.8 | 6.4 |
| 14.5 | 6.1 |
| 16.7 | 5.3 |
| 17.8 | 5.0 |
| 19.0 | 4.7 |
| 19.4 | 4.6 |
| 21.5 | 4.1 |
| 22.1 | 4.0 |
| 24.0 | 3.7 |
| 24.2 | 3.7 |
| 24.9 | 3.6 |
| 26.7 | 3.3 |
| 36.4 | 2.5 |
| 37.7 | 2.4 |

### Compound 2 Form O

A novel Compound 2 Form O can be identified by X-ray Powder Diffraction (XRPD), having one or more characteristic diffractions at angles (2 theta±0.2) of 3.4, 4.6, 6.8, 9.2, 10.1, 10.9, 12.0, and 20.2. In some embodiments, Compound 2 Form O can be identified by X-ray Powder Diffraction (XRPD), having one or more characteristic diffractions at angles (2 theta±0.2) of 3.4, 4.6, 6.8, 9.2, 10.1, 10.9, 12.0, and 20.2, corresponding to d-spacing (angstroms±0.2) of 25.7, 19.2, 13.0, 9.7, 8.7, 8.1, 7.4, and 4.4 (respectively).

A novel Compound 2 Form O can be identified by X-ray Powder Diffraction (XRPD), having three or more characteristic diffractions at angles (2 theta±0.2) of 3.4, 4.6, 6.8, 9.2, 10.1, 10.9, 12.0, and 20.2. In some embodiments, Compound 2 Form O can be identified by X-ray Powder Diffraction (XRPD), having three or more characteristic diffractions at angles (2 theta±0.2) of 3.4, 4.6, 6.8, 9.2, 10.1, 10.9, 12.0, and 20.2, corresponding to d-spacing (angstroms±0.2) of 25.7, 19.2, 13.0, 9.7, 8.7, 8.1, 7.4, and 4.4 (respectively).

In some embodiments, Compound 2 Form O is characterized by an X-ray powder diffraction having one or more peaks at substantially the same angles (2 theta±0.2) as of:
3.4
4.6
5.5
6.8
8.0
8.7
9.2
9.4
9.9
10.1
10.9
12.0
12.5
12.9
13.2
13.7
14.1
14.6
15.8
16.8
17.4
18.1
18.3
18.6
18.9
19.8
20.2
20.7
21.7
22.3
22.8
23.6
27.0
27.6
28.3
29.1
29.9
31.8
34.6
35.1

In some embodiments, Compound 2 Form O is characterized by an X-ray powder diffraction having one or more peaks at substantially the same angles (2 theta±0.2) and corresponding d-spacing (angstroms±0.2) of:

| 2 theta | d-spacing |
|---|---|
| 3.4 | 25.7 |
| 4.6 | 19.2 |
| 5.5 | 16.2 |
| 6.8 | 13.0 |
| 8.0 | 11.1 |
| 8.7 | 10.2 |
| 9.2 | 9.7 |
| 9.4 | 9.4 |
| 9.9 | 8.9 |
| 10.1 | 8.7 |
| 10.9 | 8.1 |
| 12.0 | 7.4 |
| 12.5 | 7.1 |
| 12.9 | 6.9 |
| 13.2 | 6.7 |
| 13.7 | 6.4 |
| 14.1 | 6.3 |
| 14.6 | 6.1 |
| 15.8 | 5.6 |
| 16.8 | 5.3 |
| 17.4 | 5.1 |
| 18.1 | 4.9 |
| 18.3 | 4.8 |
| 18.6 | 4.8 |
| 18.9 | 4.7 |
| 19.8 | 4.5 |
| 20.2 | 4.4 |
| 20.7 | 4.3 |
| 21.7 | 4.1 |
| 22.3 | 4.0 |
| 22.8 | 3.9 |
| 23.6 | 3.8 |
| 27.0 | 3.3 |
| 27.6 | 3.2 |
| 28.3 | 3.2 |
| 29.1 | 3.1 |
| 29.9 | 3.0 |
| 31.8 | 2.8 |
| 34.6 | 2.6 |
| 35.1 | 2.6 |

### Compound 2 Form P

A novel Compound 2 Form P can be identified by X-ray Powder Diffraction (XRPD), having one or more characteristic diffractions at angles (2 theta±0.2) of 4.3, 6.8, 12.3, 15.2, and 39.6. In some embodiments, Compound 2 Form P can be identified by X-ray Powder Diffraction (XRPD), having one or more characteristic diffractions at angles (2 theta±0.2) of 4.3, 6.8, 12.3, 15.2, and 39.6, corresponding to d-spacing (angstroms±0.2) of 20.6, 13.0, 7.2, 5.8, and 2.3 (respectively).

A novel Compound 2 Form P can be identified by X-ray Powder Diffraction (XRPD), having three or more characteristic diffractions at angles (2 theta±0.2) of 4.3, 6.8, 12.3, 15.2, and 39.6. In some embodiments, Compound 2 Form P can be identified by X-ray Powder Diffraction (XRPD), having three or more characteristic diffractions at angles (2 theta±0.2) of 4.3, 6.8, 12.3, 15.2, and 39.6, corresponding to d-spacing (angstroms±0.2) of 20.6, 13.0, 7.2, 5.8, and 2.3 (respectively).

In some embodiments, Compound 2 Form P is characterized by an X-ray powder diffraction having one or more peaks at substantially the same angles (2 theta±0.2) as of:
4.3
6.8
7.6
8.6
9.0
10.3
11.0
11.7
12.3
12.9
13.8
14.0
14.8
15.2
16.0
16.5
16.8
17.3
17.9
18.1
18.8
19.1
19.5
19.9
20.5
20.9
21.5
21.7
22.0
22.3
23.0
23.1
23.4
24.4
24.6
25.2
25.6
26.1
26.7
27.1
27.3
27.8
28.2
28.9
29.4
29.8
30.1
30.5
30.8
31.5
32.1
32.5
33.1
33.8
35.0
36.0
36.5
36.9
37.5
38.7
39.6

In some embodiments, Compound 2 Form P is characterized by an X-ray powder diffraction having one or more peaks at substantially the same angles (2 theta±0.2) and corresponding d-spacing (angstroms±0.2) of:

| 2 theta | d-spacing |
|---|---|
| 4.3 | 20.6 |
| 6.8 | 13.0 |
| 7.6 | 11.7 |
| 8.6 | 10.3 |
| 9.0 | 9.8 |
| 10.3 | 8.5 |
| 11.0 | 8.0 |
| 11.7 | 7.5 |
| 12.3 | 7.2 |
| 12.9 | 6.8 |
| 13.8 | 6.4 |
| 14.0 | 6.3 |
| 14.8 | 6.0 |
| 15.2 | 5.8 |
| 16.0 | 5.5 |
| 16.5 | 5.4 |
| 16.8 | 5.3 |
| 17.3 | 5.1 |
| 17.9 | 5.0 |
| 18.1 | 4.9 |
| 18.8 | 4.7 |
| 19.1 | 4.6 |
| 19.5 | 4.6 |
| 19.9 | 4.5 |
| 20.5 | 4.3 |
| 20.9 | 4.2 |
| 21.5 | 4.1 |
| 21.7 | 4.1 |
| 22.0 | 4.0 |
| 22.3 | 4.0 |
| 23.0 | 3.9 |
| 23.1 | 3.8 |
| 23.4 | 3.8 |
| 24.4 | 3.7 |
| 24.6 | 3.6 |
| 25.2 | 3.5 |
| 25.6 | 3.5 |
| 26.1 | 3.4 |
| 26.7 | 3.3 |
| 27.1 | 3.3 |
| 27.3 | 3.3 |
| 27.8 | 3.2 |
| 28.2 | 3.2 |
| 28.9 | 3.1 |
| 29.4 | 3.0 |
| 29.8 | 3.0 |
| 30.1 | 3.0 |
| 30.5 | 2.9 |
| 30.8 | 2.9 |
| 31.5 | 2.8 |
| 32.1 | 2.8 |
| 32.5 | 2.7 |
| 33.1 | 2.7 |
| 33.8 | 2.6 |
| 35.0 | 2.6 |
| 36.0 | 2.5 |
| 36.5 | 2.5 |
| 36.9 | 2.4 |
| 37.5 | 2.4 |
| 38.7 | 2.3 |
| 39.6 | 2.3 |

In some embodiments, the present disclosure provides a composition comprising amorphous and crystalline solid forms of Compound 2. In some embodiments, the composition comprises crystalline Compound 2 and amorphous Compound 2, wherein the amorphous Compound 2 is present in an amount selected from the following ranges: about 90 to about 99%, about 80 to about 90%, about 70 to about 80%, about 60 to about 70%, about 50 to about 60%, about 40 to about 50%, about 30 to about 40%, about 20 to about 30%, about 10 to about 20%, about 1 to about 10% and about 0 to about 1%.

In some embodiments, the composition comprises crystalline Compound 2 and amorphous Compound 2, wherein the crystalline Compound 2 is present in an amount selected from the following ranges: about 90 to about 99%, about 80 to about 90%, about 70 to about 80%, about 60 to about 70%, about 50 to about 60%, about 40 to about 50%, about 30 to about 40%, about 20 to about 30%, about 10 to about 20%, about 1 to about 10% and about 0 to about 1%. In some embodiments, the composition is free from (i.e., 0%) amorphous Compound 2.

In some embodiments, the present disclosure provides a composition comprising Compound 2 substantially free of impurities. As used herein, the term “substantially free of impurities” means that the composition contains no significant amount of extraneous matter. Such extraneous matter may include starting materials, residual solvents, or any other impurities that may result from the preparation of and/or isolation of Compound 2. In some embodiments, at least 90% by weight of Compound 2 is present. In some embodiments, at least 95% by weight of Compound 2 is present. In some embodiments, at least 99% by weight of Compound 2 is present.

In some embodiments, a crystalline solid form of Compound 2 is anhydrous. In some embodiments, an anhydrous crystalline solid form of Compound 2 is selected from Form G and Form O. In some embodiments, an anhydrous crystalline solid form of Compound 2 is Form G. In some embodiments, an anhydrous crystalline solid form of Compound 2 is Form O.

In some embodiments, a crystalline solid form of Compound 2 is unsolvated. In some embodiments, an unsolvated crystalline solid form of Compound 2 is selected from Form G and Form O. In some embodiments, an unsolvated crystalline solid form of Compound 2 is Form G. In some embodiments, an unsolvated crystalline solid form of Compound 2 is Form O.

In some embodiments, a crystalline solid form of Compound 2 is a solvate. As used herein, the term “solvate” refers to a solid form with a stoichiometric amount of solvent incorporated into the crystal structure. For example, a solvated crystalline solid form can comprise 0.5, 1.0, 1.5, 2.0, etc. equivalents of solvent incorporated into the crystal lattice.

In some embodiments, a crystalline solid form of Compound 2 is an acetone solvate. In some embodiments, an acetone solvate crystalline solid form of Compound 2 is Form A.

In some embodiments, a crystalline solid form of Compound 2 is an ethyl acetate solvate. In some embodiments, an ethyl acetate solvate crystalline solid form of Compound 2 is Form M.

In some embodiments, a crystalline solid form of Compound 2 is a hydrate. As used herein, the term “hydrate” refers to a solid form with a stoichiometric amount of water incorporated into the crystal structure. For example, a hydrated crystalline solid form can comprise 0.5, 1.0, 1.5, 2.0, etc. equivalents of water incorporated into the crystal lattice. In some embodiments, a hydrate crystalline solid form of Compound 2 is Form I.

In some embodiments, the present disclosure provides a pharmaceutical composition comprising a crystalline solid form of Compound 2. In some embodiments, the present disclosure provides a pharmaceutical composition comprising crystalline solid Form G of Compound 2. For example, a pharmaceutical composition can comprise and/or be obtained from the solid form of Compound 2 designated as Solid Form G of Compound 2 that produces an X-ray powder diffraction (XRPD) pattern having one or more diffractions at angles (2 theta±0.2) of 4.4, 6.8, 9.1, 15.3, and 38.8. In some embodiments, a pharmaceutical composition comprises Solid Form G of Compound 2 that is characterized by an X-ray Powder Diffraction (XRPD), having diffractions at angles (2 theta±0.2) of 4.4, 6.8, 9.1, 15.3, and 38.8, corresponding to d-spacing (angstroms±0.2) of 20.1, 13.0, 9.7, 5.8, and 2.3 (respectively). In some embodiments, a pharmaceutical composition comprises Solid Form G of Compound 2 that is characterized by an XRPD pattern having diffractions at angles (2 theta±0.2) of 4.4, 6.8, 9.1, 13.1, 15.3, 16.1, 19.6, 36.6, and 38.8. In some embodiments, a pharmaceutical composition comprises Solid Form G of Compound 2 that is characterized by an X-ray Powder Diffraction (XRPD), having diffractions at angles (2 theta±0.2) of 4.4, 6.8, 9.1, 13.1, 15.3, 16.1, 19.6, 36.6, and 38.8, corresponding to d-spacing (angstroms±0.2) of 20.1, 13.0, 9.7, 6.8, 5.8, 5.5, 4.5, 2.5, and 2.3 (respectively). In some embodiments, a pharmaceutical composition comprises Solid Form G of Compound 2 that is characterized by an XRPD pattern having diffractions at angles (2 theta±0.2) of:
4.4
6.8
7.7
8.7
9.1
10.5
11.1
11.8
13.1
13.9
14.9
15.3
16.1
16.6
16.9
17.4
18.0
18.2
18.9
19.2
19.6
20.0
20.7
21.1
21.6
21.8
22.1
22.5
23.0
23.5
24.5
25.3
25.8
26.2
26.8
27.5
27.9
28.4
28.7
29.1
29.9
31.0
32.7
33.3
34.0
35.2
36.1
36.6
38.8

In some embodiments, a pharmaceutical composition comprises Solid Form G of Compound 2 that is characterized by an XRPD pattern having diffractions at angles (2 theta 0.2) corresponding to d-spacing (angstroms±0.2) of:

| 2 theta | d-spacing |
|---|---|
| 4.4 | 20.1 |
| 6.8 | 13.0 |
| 7.7 | 11.4 |
| 8.7 | 10.1 |
| 9.1 | 9.7 |
| 10.5 | 8.4 |
| 11.1 | 7.9 |
| 11.8 | 7.5 |
| 13.1 | 6.8 |
| 13.9 | 6.4 |
| 14.9 | 5.9 |
| 15.3 | 5.8 |
| 16.1 | 5.5 |
| 16.6 | 5.3 |
| 16.9 | 5.2 |
| 17.4 | 5.1 |
| 18.0 | 4.9 |
| 18.2 | 4.9 |
| 18.9 | 4.7 |
| 19.2 | 4.6 |
| 19.6 | 4.5 |
| 20.0 | 4.4 |
| 20.7 | 4.3 |
| 21.1 | 4.2 |
| 21.6 | 4.1 |
| 21.8 | 4.1 |
| 22.1 | 4.0 |
| 22.5 | 4.0 |
| 23.0 | 3.9 |
| 23.5 | 3.8 |
| 24.5 | 3.6 |
| 25.3 | 3.5 |
| 25.8 | 3.5 |
| 26.2 | 3.4 |
| 26.8 | 3.3 |
| 27.5 | 3.2 |
| 27.9 | 3.2 |
| 28.4 | 3.1 |
| 28.7 | 3.1 |
| 29.1 | 3.1 |
| 29.9 | 3.0 |
| 31.0 | 2.9 |
| 32.7 | 2.7 |
| 33.3 | 2.7 |
| 34.0 | 2.6 |
| 35.2 | 2.6 |
| 36.1 | 2.5 |
| 36.6 | 2.5 |
| 38.8 | 2.3 |

In some embodiments, a pharmaceutical composition comprises Solid Form G of Compound 2 that is characterized by a differential scanning calorimetry (DSC) endotherm having a minima at about 120.1° C.

Pharmaceutical compositions reported herein can be combined with a pharmaceutically acceptable carrier or excipient. In some embodiments, pharmaceutical compositions reported herein can be provided in a unit dosage form container (e.g., in a vial or bag or the like). In some embodiments, pharmaceutical compositions reported herein can be provided in an oral dosage form. In some embodiments, an oral dosage form is a capsule. In some embodiments, an oral dosage form is a tablet.

In some embodiments, the present disclosure provides methods of inhibiting bromo and extra terminal (BET) bromodomains, comprising administering a solid form of Compound 2 to a subject. In some embodiments, the present disclosure provides methods of treating a disease, disorder, or condition responsive to inhibition of bromo and extra terminal (BET) bromodomains, comprising administering a solid form of Compound 2 to a subject in need thereof. In some embodiments, the disease, disorder, or condition is selected from cancer, inflammation, metabolic and neurological disorders, and infectious diseases.

In some embodiments, the present disclosure provides methods of treating cancer comprising administering a solid form of Compound 2 to a subject in need thereof. In some embodiments, the present disclosure provides methods of treating an inflammatory disorder comprising administering a solid form of Compound 2 to a subject in need thereof. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). In some embodiments, treatment is continued after symptoms have resolved, for example to prevent, delay or lessen the severity of their recurrence.

In some embodiments, the present disclosure provides a process for preparing Solid Form G of (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate (“Compound 2”) comprising suspending at least one of Form A, Form B, Form I, or Form O of Compound 2 in a solvent to provide a slurry, and maintaining the slurry for a period of time under conditions effective to generate Solid Form G of Compound 2. In some embodiments, a solvent is selected from isopropyl acetate (IPAc), methyl tert-butyl ether (MTBE), methyl isobutyl ketone (MIBK), methylene chloride/methyl tert-butyl ether (CHCl₃/MTBE). In some embodiments, a slurry is heated to a maximum temperature of about 50° C. after suspension in the solvent. In some embodiments, the process further comprises isolating Solid Form G of Compound 2 from the slurry.

In some embodiments, the present disclosure provides a process for preparing Solid Form G of (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate (“Compound 2”) comprising the step of contacting methyl tert-butyl ether with at least one of Form A, Form B, Form I, or Form O of Compound 2 in isopropyl acetate under conditions effective to generate Solid Form G of Compound 2.

In some embodiments, the present disclosure provides a composition comprising methyl tert-butyl ether, isopropyl acetate, and at least one of Form A, Form B, Form I, or Form O of (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate.

In some embodiments, the present disclosure provides a composition comprising a solid form of Compound 2 that is substantially free of other Compound 2 solid forms. In some embodiments, a composition comprising Compound 2 Form G is substantially free of other Compound 2 solid forms (e.g., Form A, Form B, Form C, Form D, Form E, Form F, Form I, Form J, Form K, Form L, Form M, Form O, and/or Form P). In some embodiments, a composition comprising Compound 2 Form G is substantially free of Compound 2 Form A, Compound 2 Form B, Compound 2 Form I, and Compound 2 Form O. In some embodiments, at least 90% by weight of Compound 2 Form G is present. In some embodiments, at least 95% by weight of Compound 2 Form G is present. In some embodiments, at least 99% by weight of Compound 2 Form G is present. In some embodiments, a composition comprising Compound 2 Form G is substantially free of amorphous Compound 2.

### Exemplary Embodiments

The following numbered embodiments, while non-limiting, are exemplary of certain aspects of the present disclosure:

Embodiment 1. A pharmaceutically acceptable salt form of (S)-(5-cyclobutoxy-2-methyl-6-(1-(piperidin-4-yl)-1H-pyrazol-4-yl)-3,4-dihydroquinolin-1(2H)-yl)(cyclopropyl)methanone (“Compound 1”).
Embodiment 2. A salt form of (S)-(5-cyclobutoxy-2-methyl-6-(1-(piperidin-4-yl)-1H-pyrazol-4-yl)-3,4-dihydroquinolin-1(2H)-yl)(cyclopropyl)methanone (“Compound 1”) selected from the group consisting of a Compound 1 salt form of fumaric acid, adipic acid, or succinic acid.
Embodiment 3. The salt form of any one of the preceding embodiments, wherein the salt form is a crystalline salt of Compound 1.
Embodiment 4. The salt form of any one of the preceding embodiments, wherein Compound 1 is a salt of succinic acid (“Compound 1 Succinate”).
Embodiment 5. The salt form of any one of the preceding embodiments, wherein the salt form is a crystalline succinate salt form of Compound 1 that is characterized by an X-ray powder diffraction (XRPD) pattern having diffractions at angles (2 theta±0.2) 5.5, 8.3, 9.8, 17.9, 22.2, 24.8, 25.6, 36.1, and 37.3.
Embodiment 6. The salt form of any one of the preceding embodiments, wherein the salt form is a crystalline succinate salt form of Compound 1 that is characterized by an X-ray Powder Diffraction (XRPD), having diffractions at angles (2 theta±0.2) of 5.5, 8.3, 9.8, 17.9, 22.2, 24.8, 25.6, 36.1, and 37.3, corresponding to d-spacing (angstroms±0.2) of 16.0, 10.7, 9.0, 5.0, 4.0, 3.6, 3.5, 2.5, and 2.4 (respectively).
Embodiment 7. The salt form of any one of the preceding embodiments, wherein the salt form is a crystalline succinate salt Form A of Compound 1, that is characterized by an X-ray powder diffraction (XRPD) pattern having diffractions at angles (2 theta±0.2) of:
5.5
8.3
9.8
10.8
12.5
12.6
13.8
14.4
14.6
16.7
17.1
17.9
18.4
18.7
19.0
19.6
20.0
20.5
20.9
21.3
21.8
22.2
22.5
23.1
23.4
24.2
24.8
25.3
25.6
26.5
26.8
27.3
28.1
28.5
29.1
29.7
30.7
32.3
32.9
33.8
34.9
36.1
37.3
   Embodiment 8. The salt form of any one of the preceding embodiments, wherein the salt form is a crystalline succinate salt Form A of Compound 1 that is characterized by an X-ray powder diffraction (XRPD) pattern having diffractions at angles (2 theta±0.2) and corresponding d-spacing (angstroms±0.2) of:

| 2 theta | d-spacing |
|---|---|
| 5.5 | 16.0 |
| 8.3 | 10.7 |
| 9.8 | 9.0 |
| 10.8 | 8.2 |
| 12.5 | 7.1 |
| 12.6 | 7.0 |
| 13.8 | 6.4 |
| 14.4 | 6.1 |
| 14.6 | 6.1 |
| 16.7 | 5.3 |
| 17.1 | 5.2 |
| 17.9 | 5.0 |
| 18.4 | 4.8 |
| 18.7 | 4.7 |
| 19.0 | 4.7 |
| 19.6 | 4.5 |
| 20.0 | 4.4 |
| 20.5 | 4.3 |
| 20.9 | 4.2 |
| 21.3 | 4.2 |
| 21.8 | 4.1 |
| 22.2 | 4.0 |
| 22.5 | 4.0 |
| 23.1 | 3.8 |
| 23.4 | 3.8 |
| 24.2 | 3.7 |
| 24.8 | 3.6 |
| 25.3 | 3.5 |
| 25.6 | 3.5 |
| 26.5 | 3.4 |
| 26.8 | 3.3 |
| 27.3 | 3.3 |
| 28.1 | 3.2 |
| 28.5 | 3.1 |
| 29.1 | 3.1 |
| 29.7 | 3.0 |
| 30.7 | 2.9 |
| 32.3 | 2.8 |
| 32.9 | 2.7 |
| 33.8 | 2.7 |
| 34.9 | 2.6 |
| 36.1 | 2.5 |
| 37.3 | 2.4 |

Embodiment 9. The salt form of any one of the preceding embodiments, wherein the salt form is a crystalline succinate salt Form A of Compound 1 that is characterized by a differential scanning calorimetry (DSC) endotherm having a minima at about 80.6° C.
Embodiment 10. The salt form of any one of the preceding embodiments, wherein the salt form is a crystalline succinate salt Form A of Compound 1 that is characterized by a thermogravimetric analysis (TGA) with a weight loss of about 0.4% between about 33.7° C. and about 70.0° C.
Embodiment 11. The salt form of any one of the preceding embodiments, wherein Compound 1 is a salt of fumaric acid (“Compound 1 Fumarate”).
Embodiment 12. The salt form of any one of the preceding embodiments, wherein the salt form is a crystalline fumarate salt form of Compound 1 that is characterized by an X-ray powder diffraction (XRPD) pattern having diffractions at angles (2 theta±0.2) of 4.0, 5.3, 7.9, 10.2, 13.3, and 21.2.
Embodiment 13. The salt form of any one of the preceding embodiments, wherein the salt form is a crystalline fumarate salt form of Compound 1 that is characterized by an X-ray Powder Diffraction (XRPD), having diffractions at angles (2 theta±0.2) of 4.0, 5.3, 7.9, 10.2, 13.3, and 21.2, corresponding to d-spacing (angstroms±0.2) of 22.1, 16.7, 11.1, 8.7, 6.7, and 4.2 (respectively).
Embodiment 14. The salt form of any one of the preceding embodiments, wherein the salt form is a crystalline fumarate salt Form A of Compound 1 that is characterized by an X-ray powder diffraction (XRPD) pattern having diffractions at angles (2 theta±0.2) of
4.0
5.3
6.6
7.9
10.2
13.3
14.8
17.2
18.6
21.2
24.1
26.5
28.4
   Embodiment 15. The salt form of any one of the preceding embodiments, wherein the salt form is a crystalline fumarate salt Form A of Compound 1 that is characterized by an X-ray powder diffraction (XRPD) pattern having diffractions at angles (2 theta±0.2) and corresponding d-spacing (angstroms±0.2) of:

| 2 theta | d-spacing |
|---|---|
| 4.0 | 22.1 |
| 5.3 | 16.7 |
| 6.6 | 13.4 |
| 7.9 | 11.1 |
| 10.2 | 8.7 |
| 13.3 | 6.7 |
| 14.8 | 6.0 |
| 17.2 | 5.2 |
| 18.6 | 4.8 |
| 21.2 | 4.2 |
| 24.1 | 3.7 |
| 26.5 | 3.4 |
| 28.4 | 3.1 |

Embodiment 16. The salt form of any one of the preceding embodiments, wherein the salt form is a crystalline fumarate salt Form A of Compound 1 that is characterized by a differential scanning calorimetry (DSC) endotherm having a minima at about 100.8° C.
Embodiment 17. The salt form of any one of the preceding embodiments, wherein the salt form is a crystalline fumarate salt Form A of Compound 1 that is characterized by a thermogravimetric analysis (TGA) with a weight loss of about 0.7% between about 34.4° C. and about 80.0° C.
Embodiment 18. The salt form of any one of the preceding embodiments, wherein the salt form is a crystalline fumarate salt form of Compound 1 that is characterized by an X-ray powder diffraction (XRPD) pattern having diffractions at angles (2 theta±0.2) of 5.4, 13.5, 16.3, 21.1, 23.5, 26.0, and 26.5.
Embodiment 19. The salt form of any one of the preceding embodiments, wherein the salt form is a crystalline fumarate salt form of Compound 1 that is characterized by an X-ray Powder Diffraction (XRPD), having diffractions at angles (2 theta±0.2) of 5.4, 13.5, 16.3, 21.1, 23.5, 26.0, and 26.5, corresponding to d-spacing (angstroms±0.2) of 16.4, 6.6, 5.4, 4.2, 3.8, 3.4, and 3.4 (respectively).
Embodiment 20. The salt form of any one of the preceding embodiments, wherein the salt form is a crystalline fumarate salt Form B of Compound 1 that is characterized by an X-ray powder diffraction (XRPD) pattern having diffractions at angles (2 theta±0.2) of
5.4
8.1
9.9
10.0
10.9
12.4
13.5
14.2
14.6
16.3
16.8
17.5
18.6
18.9
21.1
21.6
23.5
23.9
26.0
26.5
27.1
34.1
35.5
36.8
   Embodiment 21. The salt form of any one of the preceding embodiments, wherein the salt form is a crystalline fumarate salt Form B of Compound 1 that is characterized by an X-ray powder diffraction (XRPD) pattern having diffractions at angles (2 theta±0.2) and corresponding d-spacing (angstroms±0.2) of:

| 2 theta | d-spacing |
|---|---|
| 5.4 | 16.4 |
| 8.1 | 10.9 |
| 9.9 | 9.0 |
| 10.0 | 8.8 |
| 10.9 | 8.1 |
| 12.4 | 7.2 |
| 13.5 | 6.6 |
| 14.2 | 6.2 |
| 14.6 | 6.1 |
| 16.3 | 5.4 |
| 16.8 | 5.3 |
| 17.5 | 5.1 |
| 18.6 | 4.8 |
| 18.9 | 4.7 |
| 21.1 | 4.2 |
| 21.6 | 4.1 |
| 23.5 | 3.8 |
| 23.9 | 3.7 |
| 26.0 | 3.4 |
| 26.5 | 3.4 |
| 27.1 | 3.3 |
| 34.1 | 2.6 |
| 35.5 | 2.5 |
| 36.8 | 2.4 |

Embodiment 22. The salt form of any one of the preceding embodiments, wherein the salt form is a crystalline fumarate salt Form B of Compound 1 is characterized by a differential scanning calorimetry (DSC) endotherm having a minima at about 100.0° C.
Embodiment 23. The crystalline form of any one of the preceding embodiments, wherein the salt form is a crystalline fumarate salt Form B of Compound 1 is characterized by a thermogravimetric analysis (TGA) with a weight loss of about 1.1% between about 33.7° C. and about 90.0° C.
Embodiment 24. The salt form of any one of the preceding embodiments, wherein the salt form is a crystalline fumarate salt form of Compound 1 that is characterized by an X-ray powder diffraction (XRPD) pattern having diffractions at angles (2 theta±0.2) of 5.5, 8.2, 10.0, 18.1, 19.2, and 22.0.
Embodiment 25. The salt form of any one of the preceding embodiments, wherein the salt form is a crystalline fumarate salt form of Compound 1 that is characterized by an X-ray Powder Diffraction (XRPD), having diffractions at angles (2 theta±0.2) of 5.5, 8.2, 10.0, 18.1, 19.2, and 22.0, corresponding to d-spacing (angstroms±0.2) of 16.1, 10.7, 8.9, 4.9, 4.6, and 4.0 (respectively).
Embodiment 26. The salt form of any one of the preceding embodiments, wherein the salt form is a crystalline fumarate salt Form C of Compound 1 that is characterized by an X-ray powder diffraction (XRPD) pattern having diffractions at angles (2 theta±0.2) of
5.5
8.2
10.0
13.7
14.6
18.1
19.2
22.0
24.1
25.3
27.4
   Embodiment 27. The salt form of any one of the preceding embodiments, wherein the salt form is a crystalline fumarate salt Form C of Compound 1 that is characterized by an X-ray powder diffraction (XRPD) pattern having diffractions at angles (2 theta±0.2) and corresponding d-spacing (angstroms±0.2) of:

| 2 theta | d-spacing |
|---|---|
| 5.5 | 16.1 |
| 8.2 | 10.7 |
| 10.0 | 8.9 |
| 13.7 | 6.5 |
| 14.6 | 6.1 |
| 18.1 | 4.9 |
| 19.2 | 4.6 |
| 22.0 | 4.0 |
| 24.1 | 3.7 |
| 25.3 | 3.5 |
| 27.4 | 3.3 |

Embodiment 28. The salt form of any one of the preceding embodiments, wherein the salt form is a crystalline fumarate salt Form C of Compound 1 that is characterized by differential scanning calorimetry (DSC) endotherms having minima at about 40.2° C. and about 102.9° C.
Embodiment 29. The salt form of any one of the preceding embodiments, wherein the salt form is a crystalline fumarate salt Form C of Compound 1 that is characterized by a thermogravimetric analysis (TGA) with a weight loss of about 3.4% between about 61.9° C. and about 94.0° C.
Embodiment 30. The salt form of any one of the preceding embodiments, wherein Compound 1 is a salt of adipic acid (“Compound 1 Adipate”).
Embodiment 31. The salt form of any one of the preceding embodiments, wherein the salt form is a crystalline adipate salt form of Compound 1 that is characterized by an X-ray powder diffraction (XRPD) pattern having diffractions at angles (2 theta±0.2) of 7.9, 12.3, 15.7, 19.7, and 24.7.
Embodiment 32. The salt form of any one of the preceding embodiments, wherein the salt form is a crystalline adipate salt form of Compound 1 that is characterized by an X-ray Powder Diffraction (XRPD) having one or more diffractions at angles (2 theta±0.2) of 7.9, 12.3, 15.7, 19.7, and 24.7, corresponding to d-spacing (angstroms±0.2) of 11.2, 7.2, 5.6, 4.5, and 3.6 (respectively).
Embodiment 33. The salt form of any one of the preceding embodiments, wherein the salt form is a crystalline adipate salt Form A of Compound 1 that is characterized by an X-ray powder diffraction (XRPD) pattern having diffractions at angles (2 theta±0.2) of:
4.0
7.9
11.6
12.3
13.1
14.1
15.3
15.7
16.6
18.8
19.7
21.2
22.2
23.4
24.1
24.7
26.3
28.3
30.8
   Embodiment 34. The salt form of any one of the preceding embodiments, wherein the salt form is a crystalline adipate salt Form A of Compound 1 that is characterized by an X-ray powder diffraction (XRPD) pattern having diffractions at angles (2 theta±0.2) and corresponding d-spacing (angstroms±0.2) of:

| 2 theta | d-spacing |
|---|---|
| 4.0 | 22.3 |
| 7.9 | 11.2 |
| 11.6 | 7.6 |
| 12.3 | 7.2 |
| 13.1 | 6.8 |
| 14.1 | 6.3 |
| 15.3 | 5.8 |
| 15.7 | 5.6 |
| 16.6 | 5.3 |
| 18.8 | 4.7 |
| 19.7 | 4.5 |
| 21.2 | 4.2 |
| 22.2 | 4.0 |
| 23.4 | 3.8 |
| 24.1 | 3.7 |
| 24.7 | 3.6 |
| 26.3 | 3.4 |
| 28.3 | 3.2 |
| 30.8 | 2.9 |

Embodiment 35. The salt form of any one of the preceding embodiments, wherein the salt form is a crystalline adipate salt Form A of Compound 1 that is characterized by differential scanning calorimetry (DSC) endotherms having peaks at about 84.9° C. and about 99.9° C.
Embodiment 36. The salt form of any one of the preceding embodiments, wherein the salt form is a crystalline adipate salt Form A of Compound 1 that is characterized by a thermogravimetric analysis (TGA) with a weight loss of about 0.5% between about 33.2° C. and about 68.0° C.
Embodiment 37. A pharmaceutical composition comprising the salt form of any one of the preceding embodiments and a pharmaceutically acceptable carrier or excipient.
Embodiment 38. A process for preparing a salt form of (S)-(5-cyclobutoxy-2-methyl-6-(1-(piperidin-4-yl)-1H-pyrazol-4-yl)-3,4-dihydroquinolin-1(2H)-yl)(cyclopropyl)methanone (“Compound 1”), comprising contacting Compound 1 with an acid selected from the group consisting of fumaric acid, adipic acid, and succinic acid, under conditions and for a time effective to form the corresponding salt form of Compound 1.
Embodiment 39. A solid form of Compound 1 obtained by a process comprising the step of contacting Compound 1 with succinic acid under conditions and for a time effective to form a succinate salt form of Compound 1.
Embodiment 40. The solid form of embodiment 39, wherein the solid form is a crystalline form of Compound 1.
Embodiment 41. A pharmaceutical composition for oral administration comprising a crystalline succinate salt of Compound 1.
Embodiment 42. A pharmaceutical composition for oral administration, comprising a succinate salt of (S)-(5-cyclobutoxy-2-methyl-6-(1-(piperidin-4-yl)-1H-pyrazol-4-yl)-3,4-dihydroquinolin-1(2H)-yl)(cyclopropyl)methanone (“Compound 1”).
Embodiment 43. A pharmaceutical composition for oral administration, comprising a crystalline form of (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate.
Embodiment 44. Solid Form G of (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate (“Compound 2”).
Embodiment 45. The solid form of embodiment 44, wherein Solid Form G of Compound 2 is characterized by an X-ray powder diffraction (XRPD) pattern having diffractions at angles (2 theta±0.2) of 4.4, 6.8, 9.1, 15.3, and 38.8.
Embodiment 46. The solid form of any one of embodiments 44-45, wherein Solid Form G of Compound 2 is characterized by an XRPD pattern having diffractions at angles (2 theta±0.2) of 4.4, 6.8, 9.1, 13.1, 15.3, 16.1, 19.6, 36.6, and 38.8.
Embodiment 47. The solid form of any one of embodiments 44-46, wherein Solid Form G of Compound 2 is characterized by an X-ray Powder Diffraction (XRPD), having diffractions at angles (2 theta±0.2) of 4.4, 6.8, 9.1, 13.1, 15.3, 16.1, 19.6, 36.6, and 38.8, corresponding to d-spacing (angstroms±0.2) of 20.1, 13.0, 9.7, 6.8, 5.8, 5.5, 4.5, 2.5, and 2.3 (respectively).
Embodiment 48. The solid form of any one of embodiments 44-47, wherein Solid Form G of Compound 2 is characterized by an XRPD pattern having diffractions at angles (2 theta±0.2) of:
4.4
6.8
7.7
8.7
9.1
10.5
11.1
11.8
13.1
13.9
14.9
15.3
16.1
16.6
16.9
17.4
18.0
18.2
18.9
19.2
19.6
20.0
20.7
21.1
21.6
21.8
22.1
22.5
23.0
23.5
24.5
25.3
25.8
26.2
26.8
27.5
27.9
28.4
28.7
29.1
29.9
31.0
32.7
33.3
34.0
35.2
36.1
36.6
38.8
   Embodiment 49. The solid form of any one of embodiments 44-48, wherein Solid Form G of Compound 2 is characterized by an XRPD pattern having diffractions at angles (2 theta±0.2) corresponding to d-spacing (angstroms±0.2) of:

| 2 theta | d-spacing |
|---|---|
| 4.4 | 20.1 |
| 6.8 | 13.0 |
| 7.7 | 11.4 |
| 8.7 | 10.1 |
| 9.1 | 9.7 |
| 10.5 | 8.4 |
| 11.1 | 7.9 |
| 11.8 | 7.5 |
| 13.1 | 6.8 |
| 13.9 | 6.4 |
| 14.9 | 5.9 |
| 15.3 | 5.8 |
| 16.1 | 5.5 |
| 16.6 | 5.3 |
| 16.9 | 5.2 |
| 17.4 | 5.1 |
| 18.0 | 4.9 |
| 18.2 | 4.9 |
| 18.9 | 4.7 |
| 19.2 | 4.6 |
| 19.6 | 4.5 |
| 20.0 | 4.4 |
| 20.7 | 4.3 |
| 21.1 | 4.2 |
| 21.6 | 4.1 |
| 21.8 | 4.1 |
| 22.1 | 4.0 |
| 22.5 | 4.0 |
| 23.0 | 3.9 |
| 23.5 | 3.8 |
| 24.5 | 3.6 |
| 25.3 | 3.5 |
| 25.8 | 3.5 |
| 26.2 | 3.4 |
| 26.8 | 3.3 |
| 27.5 | 3.2 |
| 27.9 | 3.2 |
| 28.4 | 3.1 |
| 28.7 | 3.1 |
| 29.1 | 3.1 |
| 29.9 | 3.0 |
| 31.0 | 2.9 |
| 32.7 | 2.7 |
| 33.3 | 2.7 |
| 34.0 | 2.6 |
| 35.2 | 2.6 |
| 36.1 | 2.5 |
| 36.6 | 2.5 |
| 38.8 | 2.3 |

Embodiment 50. The solid form of any one of embodiments 44-49, wherein Solid Form G is characterized by a differential scanning calorimetry (DSC) endotherm having a minima at about 127° C.
Embodiment 51. The solid form of any one of embodiments 44-50, wherein Solid Form G is characterized by a thermogravimetric analysis (TGA) with a weight loss of about 0.2% between 21-150° C.
Embodiment 52. The solid form of any one of embodiments 44-51, wherein Solid Form G is characterized by a dynamic vapor sorption (DVS) of about 0.76% water by weight below 70% relative humidity.
Embodiment 53. A pharmaceutical composition comprising a Form G succinate salt of Compound 1 and a pharmaceutically acceptable excipient.
Embodiment 54. The pharmaceutical composition of any one of embodiments 44-53, wherein the Form G succinate salt of Compound 1 is a Solid Form G of (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate (“Compound 2”).
Embodiment 55. The pharmaceutical composition of any one of embodiments 44-54, wherein Solid Form G is characterized by an X-ray powder diffraction (XRPD) pattern having diffractions at angles (2 theta±0.2) of 4.4, 6.8, 9.1, 15.3, and 38.8.
Embodiment 56. The pharmaceutical composition of any one of embodiments 44-55, wherein Solid Form G of Compound 2 is characterized by an X-ray Powder Diffraction (XRPD), having diffractions at angles (2 theta±0.2) of 4.4, 6.8, 9.1, 15.3, and 38.8, corresponding to d-spacing (angstroms±0.2) of 20.1, 13.0, 9.7, 5.8, and 2.3 (respectively).
Embodiment 57. The pharmaceutical composition of any one of embodiments 44-56, wherein Solid Form G of Compound 2 is characterized by an XRPD pattern having diffractions at angles (2 theta±0.2) of 4.4, 6.8, 9.1, 13.1, 15.3, 16.1, 19.6, 36.6, and 38.8.
Embodiment 58. The pharmaceutical composition of any one of embodiments 44-57, wherein Solid Form G of Compound 2 is characterized by an X-ray Powder Diffraction (XRPD), having diffractions at angles (2 theta±0.2) of 4.4, 6.8, 9.1, 13.1, 15.3, 16.1, 19.6, 36.6, and 38.8, corresponding to d-spacing (angstroms±0.2) of 20.1, 13.0, 9.7, 6.8, 5.8, 5.5, 4.5, 2.5, and 2.3 (respectively).
Embodiment 59. The pharmaceutical composition of any one of embodiments 44-58, wherein Solid Form G of Compound 2 is characterized by an XRPD pattern having diffractions at angles (2 theta±0.2) of:
4.4
6.8
7.7
8.7
9.1
10.5
11.1
11.8
13.1
13.9
14.9
15.3
16.1
16.6
16.9
17.4
18.0
18.2
18.9
19.2
19.6
20.0
20.7
21.1
21.6
21.8
22.1
22.5
23.0
23.5
24.5
25.3
25.8
26.2
26.8
27.5
27.9
28.4
28.7
29.1
29.9
31.0
32.7
33.3
34.0
35.2
36.1
36.6
38.8
   Embodiment 60. The pharmaceutical composition of any one of embodiments 44-59, wherein Solid Form G of Compound 2 is characterized by an XRPD pattern having diffractions at angles (2 theta±0.2) corresponding to d-spacing (angstroms±0.2) of:

| 2 theta | d-spacing |
|---|---|
| 4.4 | 20.1 |
| 6.8 | 13.0 |
| 7.7 | 11.4 |
| 8.7 | 10.1 |
| 9.1 | 9.7 |
| 10.5 | 8.4 |
| 11.1 | 7.9 |
| 11.8 | 7.5 |
| 13.1 | 6.8 |
| 13.9 | 6.4 |
| 14.9 | 5.9 |
| 15.3 | 5.8 |
| 16.1 | 5.5 |
| 16.6 | 5.3 |
| 16.9 | 5.2 |
| 17.4 | 5.1 |
| 18.0 | 4.9 |
| 18.2 | 4.9 |
| 18.9 | 4.7 |
| 19.2 | 4.6 |
| 19.6 | 4.5 |
| 20.0 | 4.4 |
| 20.7 | 4.3 |
| 21.1 | 4.2 |
| 21.6 | 4.1 |
| 21.8 | 4.1 |
| 22.1 | 4.0 |
| 22.5 | 4.0 |
| 23.0 | 3.9 |
| 23.5 | 3.8 |
| 24.5 | 3.6 |
| 25.3 | 3.5 |
| 25.8 | 3.5 |
| 26.2 | 3.4 |
| 26.8 | 3.3 |
| 27.5 | 3.2 |
| 27.9 | 3.2 |
| 28.4 | 3.1 |
| 28.7 | 3.1 |
| 29.1 | 3.1 |
| 29.9 | 3.0 |
| 31.0 | 2.9 |
| 32.7 | 2.7 |
| 33.3 | 2.7 |
| 34.0 | 2.6 |
| 35.2 | 2.6 |
| 36.1 | 2.5 |
| 36.6 | 2.5 |
| 38.8 | 2.3 |

Embodiment 61. The pharmaceutical composition of any one of embodiments 44-60, wherein Solid Form G of Compound 2 is characterized by a differential scanning calorimetry (DSC) endotherm having a minima at about 127° C.
Embodiment 62. A process for preparing Solid Form G of (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate (“Compound 2”) comprising suspending at least one of Form A, Form B, Form I, or Form O of Compound 2 in a solvent to provide a slurry, and maintaining the slurry for a period of time under conditions effective to generate Solid Form G of Compound 2.
Embodiment 63. The process of any one of embodiments 44-62, wherein the solvent is selected from isopropyl acetate (IPAc), methyl tert-butyl ether (MTBE), methyl isobutyl ketone (MIBK), methylene chloride/methyl tert-butyl ether (CHCl₃/MTBE).
Embodiment 64. The process of any one of embodiments 44-63, wherein the slurry is heated to a maximum temperature of about 50° C. after suspension in the solvent.
Embodiment 65. The process of any one of embodiments 44-64, further comprising isolating Solid Form G of Compound 2 from the slurry.
Embodiment 66. A process for preparing Solid Form G of (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate (“Compound 2”) comprising the step of contacting methyl tert-butyl ether with at least one of Form A, Form B, Form I, or Form O of Compound 2 in isopropyl acetate under conditions effective to generate Solid Form G of Compound 2.
Embodiment 67. A composition comprising methyl tert-butyl ether, isopropyl acetate, and at least one of Form A, Form B, Form I, or Form O of (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate.
Embodiment 68. A crystalline solid form of Compound 2:

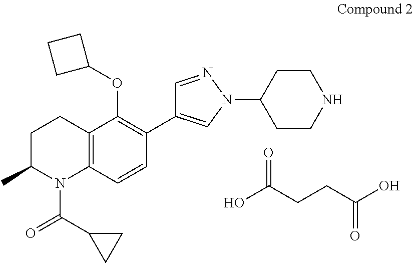

Embodiment 69. The crystalline solid form of any one of embodiments 44-68, wherein the crystalline solid form is Form A (“Solid Form A”) that is characterized by an X-ray powder diffraction (XRPD) pattern having diffractions at angles (2 theta±0.2) of 5.5, 8.3, 9.8, 17.9, 22.2, 24.8, 25.6, 36.1, and 37.3.
Embodiment 70. The crystalline solid form of any one of embodiments 44-69, wherein the crystalline solid form is Solid Form A that is characterized by X-ray Powder Diffraction (XRPD), having diffractions at angles (2 theta±0.2) of 5.5, 8.3, 9.8, 17.9, 22.2, 24.8, 25.6, 36.1, and 37.3, corresponding to d-spacing (angstroms±0.2) of 16.0, 10.7, 9.0, 5.0, 4.0, 3.6, 3.5, 2.5, and 2.4 (respectively).
Embodiment 71. The crystalline solid form of any one of embodiments 44-70, wherein the crystalline solid form is Solid Form A that is characterized by an XRPD pattern having diffractions at angles (2 theta±0.2) of
5.5
8.3
9.8
10.8
12.5
12.6
13.8
14.4
14.6
16.7
17.1
17.9
18.4
18.7
19.0
19.6
20.0
20.5
20.9
21.3
21.8
22.2
22.5
23.1
23.4
24.2
24.8
25.3
25.6
26.5
26.8
27.3
28.1
28.5
29.1
29.7
30.7
32.3
32.9
33.8
34.9
36.1
37.3
   Embodiment 72. The crystalline solid form of any one of embodiments 44-71, wherein the crystalline solid form is Solid Form A that is characterized by an XRPD pattern having diffractions at angles (2 theta±0.2) corresponding to d-spacing (angstroms±0.2) of:

| 2 theta | d-spacing |
|---|---|
| 5.5 | 16.0 |
| 8.3 | 10.7 |
| 9.8 | 9.0 |
| 10.8 | 8.2 |
| 12.5 | 7.1 |
| 12.6 | 7.0 |
| 13.8 | 6.4 |
| 14.4 | 6.1 |
| 14.6 | 6.1 |
| 16.7 | 5.3 |
| 17.1 | 5.2 |
| 17.9 | 5.0 |
| 18.4 | 4.8 |
| 18.7 | 4.7 |
| 19.0 | 4.7 |
| 19.6 | 4.5 |
| 20.0 | 4.4 |
| 20.5 | 4.3 |
| 20.9 | 4.2 |
| 21.3 | 4.2 |
| 21.8 | 4.1 |
| 22.2 | 4.0 |
| 22.5 | 4.0 |
| 23.1 | 3.8 |
| 23.4 | 3.8 |
| 24.2 | 3.7 |
| 24.8 | 3.6 |
| 25.3 | 3.5 |
| 25.6 | 3.5 |
| 26.5 | 3.4 |
| 26.8 | 3.3 |
| 27.3 | 3.3 |
| 28.1 | 3.2 |
| 28.5 | 3.1 |
| 29.1 | 3.1 |
| 29.7 | 3.0 |
| 30.7 | 2.9 |
| 32.3 | 2.8 |
| 32.9 | 2.7 |
| 33.8 | 2.7 |
| 34.9 | 2.6 |
| 36.1 | 2.5 |
| 37.3 | 2.4 |

Embodiment 73. The crystalline solid form of any one of embodiments 44-72, wherein the crystalline solid form is Form B (“Solid Form B”) that is characterized by an X-ray powder diffraction (XRPD) pattern having diffractions at angles (2 theta±0.2) of 4.7, 5.9, 8.7, 11.0, 17.2, and 20.4.
Embodiment 74. The crystalline solid form of any one of embodiments 44-73, wherein the crystalline solid form is Solid Form B that is characterized by X-ray Powder Diffraction (XRPD), having diffractions at angles (2 theta±0.2) of 4.7, 5.9, 8.7, 11.0, 17.2, and 20.4, corresponding to d-spacing (angstroms±0.2) of 18.7, 15.1, 10.1, 8.1, 5.1, and 4.4 (respectively).
Embodiment 75. The crystalline solid form of any one of embodiments 44-74, wherein the solid form is Solid Form B that is characterized by an XRPD pattern having diffractions at angles (2 theta±0.2) of:
4.7
5.9
8.7
9.7
10.3
11.0
11.7
13.8
14.5
16.2
17.2
17.7
18.5
18.8
19.1
20.4
20.6
21.2
21.6
22.6
23.8
24.3
24.9
25.2
26.1
26.9
27.7
28.5
30.0
32.7
33.6
35.1
   Embodiment 76. The crystalline solid form of any one of embodiments 44-75, wherein the crystalline solid form is Solid Form B that is characterized by an XRPD pattern having diffractions at angles (2 theta±0.2) corresponding to d-spacing (angstroms±0.2) of:

| 2 theta | d-spacing |
|---|---|
| 4.7 | 18.7 |
| 5.9 | 15.1 |
| 8.7 | 10.1 |
| 9.7 | 9.1 |
| 10.3 | 8.6 |
| 11.0 | 8.1 |
| 11.7 | 7.5 |
| 13.8 | 6.4 |
| 14.5 | 6.1 |
| 16.2 | 5.5 |
| 17.2 | 5.1 |
| 17.7 | 5.0 |
| 18.5 | 4.8 |
| 18.8 | 4.7 |
| 19.1 | 4.6 |
| 20.4 | 4.4 |
| 20.6 | 4.3 |
| 21.2 | 4.2 |
| 21.6 | 4.1 |
| 22.6 | 3.9 |
| 23.8 | 3.7 |
| 24.3 | 3.7 |
| 24.9 | 3.6 |
| 25.2 | 3.5 |
| 26.1 | 3.4 |
| 26.9 | 3.3 |
| 27.7 | 3.2 |
| 28.5 | 3.1 |
| 30.0 | 3.0 |
| 32.7 | 2.7 |
| 33.6 | 2.7 |
| 35.1 | 2.6 |

Embodiment 77. The crystalline solid form of any one of embodiments 44-76, wherein the crystalline solid form is Form C (“Solid Form C”) that is characterized by an X-ray powder diffraction (XRPD) pattern having diffractions at angles (2 theta±0.2) of 5.4, 8.1, 10.1, 10.9, 16.4, 17.7, and 34.3.
Embodiment 78. The crystalline solid form of any one of embodiments 44-77, wherein the crystalline solid form is Solid Form C that is characterized by X-ray Powder Diffraction (XRPD), having diffractions at angles (2 theta±0.2) of 5.4, 8.1, 10.1, 10.9, 16.4, 17.7, and 34.3, corresponding to d-spacing (angstroms±0.2) of 16.3, 10.9, 8.7, 8.1, 5.4, 5.0, and 2.6 (respectively).
Embodiment 79. The crystalline solid form of any one of embodiments 44-78, wherein the solid form is Solid Form C that is characterized by an XRPD pattern having diffractions at angles (2 theta±0.2) of:
5.4
8.1
9.9
10.1
10.9
12.4
14.2
14.8
16.4
17.1
17.7
18.6
19.3
19.9
20.7
21.2
21.8
22.6
23.2
24.2
24.6
25.2
26.0
27.3
29.3
30.0
34.3
37.5
   Embodiment 80. The crystalline solid form of any one of embodiments 44-79, wherein the crystalline solid form is Solid Form C that is characterized by an XRPD pattern having diffractions at angles (2 theta±0.2) corresponding to d-spacing (angstroms±0.2) of:

| 2 theta | d-spacing |
|---|---|
| 5.4 | 16.3 |
| 8.1 | 10.9 |
| 9.9 | 8.9 |
| 10.1 | 8.7 |
| 10.9 | 8.1 |
| 12.4 | 7.1 |
| 14.2 | 6.2 |
| 14.8 | 6.0 |
| 16.4 | 5.4 |
| 17.1 | 5.2 |
| 17.7 | 5.0 |
| 18.6 | 4.8 |
| 19.3 | 4.6 |
| 19.9 | 4.5 |
| 20.7 | 4.3 |
| 21.2 | 4.2 |
| 21.8 | 4.1 |
| 22.6 | 3.9 |
| 23.2 | 3.8 |
| 24.2 | 3.7 |
| 24.6 | 3.6 |
| 25.2 | 3.5 |
| 26.0 | 3.4 |
| 27.3 | 3.3 |
| 29.3 | 3.0 |
| 30.0 | 3.0 |
| 34.3 | 2.6 |
| 37.5 | 2.4 |

Embodiment 81. The crystalline solid form of any one of embodiments 44-80, wherein the crystalline solid form is Form D (“Solid Form D”) that is characterized by an X-ray powder diffraction (XRPD) pattern having diffractions at angles (2 theta±0.2) of 5.4, 14.7, 18.5, 21.8, and 38.6.
Embodiment 82. The crystalline solid form of any one of embodiments 44-81, wherein the crystalline solid form is Solid Form D that is characterized by an X-ray Powder Diffraction (XRPD), having diffractions at angles (2 theta±0.2) of 5.4, 14.7, 18.5, 21.8, and 38.6 corresponding to d-spacing (angstroms±0.2) of 16.2, 6.0, 4.8, 4.1, and 2.3 (respectively).
Embodiment 83. The crystalline solid form of any one of embodiments 44-82, wherein the crystalline solid form is Solid Form D that is characterized by an XRPD pattern having diffractions at angles (2 theta±0.2) of:
5.4
8.1
9.8
10.0
10.8
12.4
13.6
14.2
14.7
16.8
17.3
18.5
19.1
19.9
20.5
20.9
21.8
23.5
24.1
25.1
25.7
26.1
27.6
29.6
33.4
35.8
38.6
   Embodiment 84. The crystalline solid form of any one of embodiments 44-83, wherein the crystalline solid form is Solid Form D that is characterized by an XRPD pattern having diffractions at angles (2 theta±0.2) corresponding to d-spacing (angstroms±0.2) of:

| 2 theta | d-spacing |
|---|---|
| 5.4 | 16.2 |
| 8.1 | 10.9 |
| 9.8 | 9.0 |
| 10.0 | 8.9 |
| 10.8 | 8.2 |
| 12.4 | 7.2 |
| 13.6 | 6.5 |
| 14.2 | 6.2 |
| 14.7 | 6.0 |
| 16.8 | 5.3 |
| 17.3 | 5.1 |
| 18.5 | 4.8 |
| 19.1 | 4.7 |
| 19.9 | 4.5 |
| 20.5 | 4.3 |
| 20.9 | 4.2 |
| 21.8 | 4.1 |
| 23.5 | 3.8 |
| 24.1 | 3.7 |
| 25.1 | 3.5 |
| 25.7 | 3.5 |
| 26.1 | 3.4 |
| 27.6 | 3.2 |
| 29.6 | 3.0 |
| 33.4 | 2.7 |
| 35.8 | 2.5 |
| 38.6 | 2.3 |

Embodiment 85. The crystalline solid form of any one of embodiments 44-84, wherein the crystalline solid form is Form E (“Solid Form E”) that is characterized by an X-ray powder diffraction (XRPD) pattern having diffractions at angles (2 theta±0.2) of 5.5, 11.3, 13.0, 16.3, and 20.3.
Embodiment 86. The crystalline solid form of any one of embodiments 44-85, wherein the crystalline solid form is Solid Form E that is characterized by an X-ray Powder Diffraction (XRPD), having diffractions at angles (2 theta±0.2) of 5.5, 11.3, 13.0, 16.3, and 20.3, corresponding to d-spacing (angstroms±0.2) of 16.0, 7.8, 6.8, 5.4, and 4.4 (respectively).
Embodiment 87. The crystalline solid form of any one of embodiments 44-86, wherein the crystalline solid form is Solid Form E that is characterized by an XRPD pattern having diffractions at angles (2 theta±0.2) of:
5.5
8.3
9.8
10.1
10.7
11.3
12.2
13.0
13.8
14.1
15.0
16.3
16.5
17.3
18.2
18.6
19.0
19.6
20.3
20.9
21.2
22.1
22.9
23.6
24.2
26.2
27.2
28.1
30.4
32.8
35.3
36.3
37.0
   Embodiment 88. The crystalline solid form of any one of embodiments 44-87, wherein the crystalline solid form is Solid Form E that is characterized by an XRPD pattern having diffractions at angles (2 theta±0.2) corresponding to d-spacing (angstroms±0.2) of:

| 2 theta | d-spacing |
|---|---|
| 5.5 | 16.0 |
| 8.3 | 10.7 |
| 9.8 | 9.1 |
| 10.1 | 8.8 |
| 10.7 | 8.3 |
| 11.3 | 7.8 |
| 12.2 | 7.2 |
| 13.0 | 6.8 |
| 13.8 | 6.4 |
| 14.1 | 6.3 |
| 15.0 | 5.9 |
| 16.3 | 5.4 |
| 16.5 | 5.4 |
| 17.3 | 5.1 |
| 18.2 | 4.9 |
| 18.6 | 4.8 |
| 19.0 | 4.7 |
| 19.6 | 4.5 |
| 20.3 | 4.4 |
| 20.9 | 4.3 |
| 21.2 | 4.2 |
| 22.1 | 4.0 |
| 22.9 | 3.9 |
| 23.6 | 3.8 |
| 24.2 | 3.7 |
| 26.2 | 3.4 |
| 27.2 | 3.3 |
| 28.1 | 3.2 |
| 30.4 | 2.9 |
| 32.8 | 2.7 |
| 35.3 | 2.5 |
| 36.3 | 2.5 |
| 37.0 | 2.4 |

Embodiment 89. The crystalline solid form of any one of embodiments 44-88, wherein the crystalline solid form is Form F (“Solid Form F”) that is characterized by an X-ray powder diffraction (XRPD) pattern having diffractions at angles (2 theta±0.2) of 13.6, 14.2, 16.3, 21.8, and 26.7.
Embodiment 90. The crystalline solid form of any one of embodiments 44-89, wherein the crystalline solid form is Solid Form F that is characterized by X-ray Powder Diffraction (XRPD), having diffractions at angles (2 theta±0.2) of 13.6, 14.2, 16.3, 21.8, and 26.7, corresponding to d-spacing (angstroms±0.2) of 6.5, 6.2, 5.4, 4.1, and 3.3 (respectively).
Embodiment 91. The crystalline solid form of any one of embodiments 44-90, wherein the crystalline solid form is Solid Form F that is characterized by an XRPD pattern having diffractions at angles (2 theta±0.2) of:
5.5
8.2
9.7
9.9
10.7
12.3
13.6
14.2
14.6
16.3
16.8
18.6
18.9
19.1
19.5
20.3
20.8
21.0
21.8
22.9
23.5
23.8
24.1
26.1
26.7
27.4
29.4
   Embodiment 92. The crystalline solid form of any one of embodiments 44-91, wherein the crystalline solid form is Solid Form F that is characterized by an XRPD pattern having diffractions at angles (2 theta±0.2) corresponding to d-spacing (angstroms±0.2) of:

| 2 theta | d-spacing |
|---|---|
| 5.5 | 16.1 |
| 8.2 | 10.8 |
| 9.7 | 9.1 |
| 9.9 | 8.9 |
| 10.7 | 8.3 |
| 12.3 | 7.2 |
| 13.6 | 6.5 |
| 14.2 | 6.2 |
| 14.6 | 6.0 |
| 16.3 | 5.4 |
| 16.8 | 5.3 |
| 18.6 | 4.8 |
| 18.9 | 4.7 |
| 19.1 | 4.6 |
| 19.5 | 4.6 |
| 20.3 | 4.4 |
| 20.8 | 4.3 |
| 21.0 | 4.2 |
| 21.8 | 4.1 |
| 22.9 | 3.9 |
| 23.5 | 3.8 |
| 23.8 | 3.7 |
| 24.1 | 3.7 |
| 26.1 | 3.4 |
| 26.7 | 3.3 |
| 27.4 | 3.3 |
| 29.4 | 3.0 |

Embodiment 93. The crystalline solid form of any one of embodiments 44-92, wherein the crystalline solid form is Form I (“Solid Form I”) that is characterized by an X-ray powder diffraction (XRPD) pattern having diffractions at angles (2 theta±0.2) of 3.4, 5.9, 9.2, 10.3, 11.0, and 25.4.
Embodiment 94. The crystalline solid form of any one of embodiments 44-93, wherein the crystalline solid form is Solid Form I that is characterized by an X-ray Powder Diffraction (XRPD), having diffractions at angles (2 theta±0.2) of 3.4, 5.9, 9.2, 10.3, 11.0, and 25.4, corresponding to d-spacing (angstroms±0.2) of 25.8, 15.0, 9.7, 8.6, 8.0, and 3.5 (respectively).
Embodiment 95. The crystalline solid form of any one of embodiments 44-94, wherein the crystalline solid form is Solid Form I that is characterized by an XRPD pattern having diffractions at angles (2 theta±0.2) of
3.4
5.9
6.7
8.0
8.8
9.2
9.5
9.8
10.1
10.3
10.8
11.0
12.5
12.9
13.2
13.9
14.5
15.7
16.8
17.4
18.3
18.9
19.8
20.7
21.4
23.6
25.4
27.0
28.3
29.1
29.9
   Embodiment 96. The crystalline solid form of any one of embodiments 44-95, wherein the crystalline solid form is Solid Form I that is characterized by an XRPD pattern having diffractions at angles (2 theta±0.2) corresponding to d-spacing (angstroms±0.2) of:

| 2 theta | d-spacing |
|---|---|
| 3.4 | 25.8 |
| 5.9 | 15.0 |
| 6.7 | 13.1 |
| 8.0 | 11.1 |
| 8.8 | 10.0 |
| 9.2 | 9.7 |
| 9.5 | 9.4 |
| 9.8 | 9.0 |
| 10.1 | 8.8 |
| 10.3 | 8.6 |
| 10.8 | 8.2 |
| 11.0 | 8.0 |
| 12.5 | 7.1 |
| 12.9 | 6.9 |
| 13.2 | 6.7 |
| 13.9 | 6.4 |
| 14.5 | 6.1 |
| 15.7 | 5.6 |
| 16.8 | 5.3 |
| 17.4 | 5.1 |
| 18.3 | 4.8 |
| 18.9 | 4.7 |
| 19.8 | 4.5 |
| 20.7 | 4.3 |
| 21.4 | 4.2 |
| 23.6 | 3.8 |
| 25.4 | 3.5 |
| 27.0 | 3.3 |
| 28.3 | 3.2 |
| 29.1 | 3.1 |
| 29.9 | 3.0 |

Embodiment 97. The crystalline solid form of any one of embodiments 44-96, wherein the crystalline solid form is Form J (“Solid Form J”) that is characterized by an X-ray powder diffraction (XRPD) pattern having diffractions at angles (2 theta±0.2) of 5.4, 8.1, 12.4, 13.5, 15.6, 21.0, and 21.7.
Embodiment 98. The crystalline solid form of any one of embodiments 44-97, wherein the crystalline solid form is Solid Form J that is characterized by an X-ray Powder Diffraction (XRPD), having diffractions at angles (2 theta±0.2) of 5.4, 8.1, 12.4, 13.5, 15.6, 21.0, and 21.7, corresponding to d-spacing (angstroms±0.2) of 16.3, 10.9, 7.1, 6.5, 5.7, 4.2, and 4.1 (respectively).
Embodiment 99. The crystalline solid form of any one of embodiments 44-98, wherein the crystalline solid form is Solid Form J that is characterized by an XRPD pattern having diffractions at angles (2 theta±0.2) of:
5.4
8.1
9.8
10.0
10.8
12.4
13.5
15.6
18.7
19.0
21.0
21.7
23.5
35.6
38.5
   Embodiment 100. The crystalline solid form of any one of embodiments 44-99, wherein the crystalline solid form is Solid Form J that is characterized by an XRPD pattern having diffractions at angles (2 theta±0.2) corresponding to d-spacing (angstroms±0.2) of:

| 2 theta | d-spacing |
|---|---|
| 5.4 | 16.3 |
| 8.1 | 10.9 |
| 9.8 | 9.0 |
| 10.0 | 8.8 |
| 10.8 | 8.2 |
| 12.4 | 7.1 |
| 13.5 | 6.5 |
| 15.6 | 5.7 |
| 18.7 | 4.7 |
| 19.0 | 4.7 |
| 21.0 | 4.2 |
| 21.7 | 4.1 |
| 23.5 | 3.8 |
| 35.6 | 2.5 |
| 38.5 | 2.3 |

Embodiment 101. The crystalline solid form of any one of embodiments 44-100, wherein the crystalline solid form is Form K (“Solid Form K”) that is characterized by an X-ray powder diffraction (XRPD) pattern having diffractions at angles (2 theta±0.2) of 5.5, 8.7, 9.7, 22.2, and 24.4.
Embodiment 102. The crystalline solid form of any one of embodiments 44-101, wherein the crystalline solid form is Solid Form K that is characterized by an X-ray Powder Diffraction (XRPD), having diffractions at angles (2 theta±0.2) of 5.5, 8.7, 9.7, 22.2, and 24.4, corresponding to d-spacing (angstroms±0.2) of 15.9, 10.2, 9.1, 4.0, and 3.7 (respectively).
Embodiment 103. The crystalline solid form of any one of embodiments 44-102, wherein the crystalline solid form is Solid Form K that is characterized by an XRPD pattern having diffractions at angles (2 theta±0.2) of:
5.5
8.3
8.7
9.7
10.9
14.5
16.8
17.6
18.7
20.5
22.2
24.4
   Embodiment 104. The crystalline solid form of any one of embodiments 44-103, wherein the crystalline solid form is Solid Form K that is characterized by an XRPD pattern having diffractions at angles (2 theta±0.2) corresponding to d-spacing (angstroms±0.2) of:

| 2 theta | d-spacing |
|---|---|
| 5.5 | 15.9 |
| 8.3 | 10.7 |
| 8.7 | 10.2 |
| 9.7 | 9.1 |
| 10.9 | 8.1 |
| 14.5 | 6.1 |
| 16.8 | 5.3 |
| 17.6 | 5.0 |
| 18.7 | 4.7 |
| 20.5 | 4.3 |
| 22.2 | 4.0 |
| 24.4 | 3.7 |

Embodiment 105. The crystalline solid form of any one of embodiments 44-104, wherein the crystalline solid form is Form L (“Solid Form L”) characterized by an X-ray powder diffraction (XRPD) pattern having diffractions at angles (2 theta±0.2) of 5.4, 9.8, 12.6, 17.8, 18.9, 21.0, 22.2, and 29.2.
Embodiment 106. The crystalline solid form of any one of embodiments 44-105, wherein the crystalline solid form is Solid Form L that is characterized by an X-ray Powder Diffraction (XRPD), having diffractions at angles (2 theta±0.2) of 5.5, 9.8, 12.6, 17.8, 18.9, 21.0, 22.2, and 29.2, corresponding to d-spacing (angstroms±0.2) of 16.0, 9.0, 7.0, 5.0, 4.7, 4.2, 4.0, and 3.1 (respectively).
Embodiment 107. The crystalline solid form of any one of embodiments 44-106, wherein the crystalline solid form is Solid Form L that is characterized by an XRPD pattern having diffractions at angles (2 theta±0.2) of:
5.5
8.3
9.8
11.0
12.6
13.9
14.6
16.6
17.0
17.8
18.7
18.9
19.5
19.8
21.0
22.2
22.8
23.7
24.1
25.0
25.8
26.5
26.8
28.2
29.2
   Embodiment 108. The crystalline solid form of any one of embodiments 44-107, wherein the crystalline solid form is Solid Form L that is characterized by an XRPD pattern having diffractions at angles (2 theta±0.2) corresponding to d-spacing (angstroms±0.2) of:

| 2 theta | d-spacing |
|---|---|
| 5.5 | 16.0 |
| 8.3 | 10.6 |
| 9.8 | 9.0 |
| 11.0 | 8.1 |
| 12.6 | 7.0 |
| 13.9 | 6.4 |
| 14.6 | 6.1 |
| 16.6 | 5.3 |
| 17.0 | 5.2 |
| 17.8 | 5.0 |
| 18.7 | 4.7 |
| 18.9 | 4.7 |
| 19.5 | 4.6 |
| 19.8 | 4.5 |
| 21.0 | 4.2 |
| 22.2 | 4.0 |
| 22.8 | 3.9 |
| 23.7 | 3.8 |
| 24.1 | 3.7 |
| 25.0 | 3.6 |
| 25.8 | 3.5 |
| 26.5 | 3.4 |
| 26.8 | 3.3 |
| 28.2 | 3.2 |
| 29.2 | 3.1 |

Embodiment 109. The crystalline solid form of any one of embodiments 44-108, wherein the crystalline solid form is Form M (“Solid Form M”) that is characterized by an X-ray powder diffraction (XRPD) pattern having diffractions at angles (2 theta±0.2) of 5.5, 8.3, 14.5, 19.4, 22.1, 24.9, and 37.7.
Embodiment 110. The crystalline solid form of any one of embodiments 44-109, wherein the crystalline solid form is Solid Form M that is characterized by an X-ray Powder Diffraction (XRPD), having diffractions at angles (2 theta±0.2) of 5.5, 8.3, 14.5, 19.4, 22.1, 24.9, and 37.7, corresponding to d-spacing (angstroms±0.2) of 16.0, 10.7, 6.1, 4.6, 4.0, 3.6, and 2.4 (respectively).
Embodiment 111. The crystalline solid form of any one of embodiments 44-110, wherein the crystalline solid form is Solid Form M that is characterized by an XRPD pattern having diffractions at angles (2 theta±0.2) of:
5.5
8.3
9.8
10.8
12.4
13.8
14.5
16.7
17.8
19.0
19.4
21.5
22.1
24.0
24.2
24.9
26.7
36.4
37.7
   Embodiment 112. The crystalline solid form of any one of embodiments 44-111, wherein the crystalline solid form is Solid Form M that is characterized by an XRPD pattern having diffractions at angles (2 theta±0.2) corresponding to d-spacing (angstroms±0.2) of:

| 2 theta | d-spacing |
|---|---|
| 5.5 | 16.0 |
| 8.3 | 10.7 |
| 9.8 | 9.0 |
| 10.8 | 8.2 |
| 12.4 | 7.1 |
| 13.8 | 6.4 |
| 14.5 | 6.1 |
| 16.7 | 5.3 |
| 17.8 | 5.0 |
| 19.0 | 4.7 |
| 19.4 | 4.6 |
| 21.5 | 4.1 |
| 22.1 | 4.0 |
| 24.0 | 3.7 |
| 24.2 | 3.7 |
| 24.9 | 3.6 |
| 26.7 | 3.3 |
| 36.4 | 2.5 |
| 37.7 | 2.4 |

Embodiment 113. The crystalline solid form of any one of embodiments 44-112, wherein the crystalline solid form is Form O (“Solid Form O”) that is characterized by an X-ray powder diffraction (XRPD) pattern having diffractions at angles (2 theta±0.2) of 3.4, 4.6, 6.8, 9.2, 10.1, 10.9, 12.0, and 20.2.
Embodiment 114. The crystalline solid form of any one of embodiments 44-113, wherein the crystalline solid form is Solid Form O that is characterized by an X-ray Powder Diffraction (XRPD) having diffractions at angles (2 theta±0.2) of 3.4, 4.6, 6.8, 9.2, 10.1, 10.9, 12.0, and 20.2, corresponding to d-spacing (angstroms±0.2) of 25.7, 19.2, 13.0, 9.7, 8.7, 8.1, 7.4, and 4.4 (respectively).
Embodiment 115. The crystalline solid form of any one of embodiments 44-114, wherein the crystalline solid form is Solid Form O that is characterized by an XRPD pattern having diffractions at angles (2 theta±0.2) of:
3.4
4.6
5.5
6.8
8.0
8.7
9.2
9.4
9.9
10.1
10.9
12.0
12.5
12.9
13.2
13.7
14.1
14.6
15.8
16.8
17.4
18.1
18.3
18.6
18.9
19.8
20.2
20.7
21.7
22.3
22.8
23.6
27.0
27.6
28.3
29.1
29.9
31.8
34.6
35.1
   Embodiment 116. The crystalline solid form of any one of embodiments 44-115, wherein the crystalline solid form is Solid Form O that is characterized by an XRPD pattern having diffractions at angles (2 theta±0.2) corresponding to d-spacing (angstroms±0.2) of:

| 2 theta | d-spacing |
|---|---|
| 3.4 | 25.7 |
| 4.6 | 19.2 |
| 5.5 | 16.2 |
| 6.8 | 13.0 |
| 8.0 | 11.1 |
| 8.7 | 10.2 |
| 9.2 | 9.7 |
| 9.4 | 9.4 |
| 9.9 | 8.9 |
| 10.1 | 8.7 |
| 10.9 | 8.1 |
| 12.0 | 7.4 |
| 12.5 | 7.1 |
| 12.9 | 6.9 |
| 13.2 | 6.7 |
| 13.7 | 6.4 |
| 14.1 | 6.3 |
| 14.6 | 6.1 |
| 15.8 | 5.6 |
| 16.8 | 5.3 |
| 17.4 | 5.1 |
| 18.1 | 4.9 |
| 18.3 | 4.8 |
| 18.6 | 4.8 |
| 18.9 | 4.7 |
| 19.8 | 4.5 |
| 20.2 | 4.4 |
| 20.7 | 4.3 |
| 21.7 | 4.1 |
| 22.3 | 4.0 |
| 22.8 | 3.9 |
| 23.6 | 3.8 |
| 27.0 | 3.3 |
| 27.6 | 3.2 |
| 28.3 | 3.2 |
| 29.1 | 3.1 |
| 29.9 | 3.0 |
| 31.8 | 2.8 |
| 34.6 | 2.6 |
| 35.1 | 2.6 |

Embodiment 117. The crystalline solid form of any one of embodiments 44-116, wherein the crystalline solid form is Form P (“Solid Form P”) that is characterized by an X-ray powder diffraction (XRPD) pattern having diffractions at angles (2 theta±0.2) of 4.3, 6.8, 12.3, 15.2, and 39.6.
Embodiment 118. The crystalline solid form of any one of embodiments 44-117, wherein the crystalline solid form is Solid Form P that is characterized by an X-ray Powder Diffraction (XRPD), having diffractions at angles (2 theta±0.2) of 4.3, 6.8, 12.3, 15.2, and 39.6, corresponding to d-spacing (angstroms±0.2) of 20.6, 13.0, 7.2, 5.8, and 2.3 (respectively).
Embodiment 119. The crystalline solid form of any one of embodiments 44-118, wherein the crystalline solid form is Solid Form P that is characterized by an XRPD pattern having diffractions at angles (2 theta±0.2) of:
4.3
6.8
7.6
8.6
9.0
10.3
11.0
11.7
12.3
12.9
13.8
14.0
14.8
15.2
16.0
16.5
16.8
17.3
17.9
18.1
18.8
19.1
19.5
19.9
20.5
20.9
21.5
21.7
22.0
22.3
23.0
23.1
23.4
24.4
24.6
25.2
25.6
26.1
26.7
27.1
27.3
27.8
28.2
28.9
29.4
29.8
30.1
30.5
30.8
31.5
32.1
32.5
33.1
33.8
35.0
36.0
36.5
36.9
37.5
38.7
39.6
   Embodiment 120. The crystalline solid form of any one of embodiments 44-119, wherein the crystalline solid form is Solid Form P that is characterized by an XRPD pattern having diffractions at angles (2 theta±0.2) corresponding to d-spacing (angstroms±0.2) of:

| 2 theta | d-spacing |
|---|---|
| 4.3 | 20.6 |
| 6.8 | 13.0 |
| 7.6 | 11.7 |
| 8.6 | 10.3 |
| 9.0 | 9.8 |
| 10.3 | 8.5 |
| 11.0 | 8.0 |
| 11.7 | 7.5 |
| 12.3 | 7.2 |
| 12.9 | 6.8 |
| 13.8 | 6.4 |
| 14.0 | 6.3 |
| 14.8 | 6.0 |
| 15.2 | 5.8 |
| 16.0 | 5.5 |
| 16.5 | 5.4 |
| 16.8 | 5.3 |
| 17.3 | 5.1 |
| 17.9 | 5.0 |
| 18.1 | 4.9 |
| 18.8 | 4.7 |
| 19.1 | 4.6 |
| 19.5 | 4.6 |
| 19.9 | 4.5 |
| 20.5 | 4.3 |
| 20.9 | 4.2 |
| 21.5 | 4.1 |
| 21.7 | 4.1 |
| 22.0 | 4.0 |
| 22.3 | 4.0 |
| 23.0 | 3.9 |
| 23.1 | 3.8 |
| 23.4 | 3.8 |
| 24.4 | 3.7 |
| 24.6 | 3.6 |
| 25.2 | 3.5 |
| 25.6 | 3.5 |
| 26.1 | 3.4 |
| 26.7 | 3.3 |
| 27.1 | 3.3 |
| 27.3 | 3.3 |
| 27.8 | 3.2 |
| 28.2 | 3.2 |
| 28.9 | 3.1 |
| 29.4 | 3.0 |
| 29.8 | 3.0 |
| 30.1 | 3.0 |
| 30.5 | 2.9 |
| 30.8 | 2.9 |
| 31.5 | 2.8 |
| 32.1 | 2.8 |
| 32.5 | 2.7 |
| 33.1 | 2.7 |
| 33.8 | 2.6 |
| 35.0 | 2.6 |
| 36.0 | 2.5 |
| 36.5 | 2.5 |
| 36.9 | 2.4 |
| 37.5 | 2.4 |
| 38.7 | 2.3 |
| 39.6 | 2.3 |

Embodiment 121. A crystalline solid form of (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate (“Compound 2”).
Embodiment 122. A crystalline solid Form G of (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate (“Compound 2”).
Embodiment 123. A pharmaceutical composition comprising a crystalline solid form of (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate (“Compound 2”).
Embodiment 124. A pharmaceutical composition comprising crystalline Solid Form G of (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate (“Compound 2”).
Embodiment 125. A pharmaceutical composition for oral administration comprising the crystalline solid Form G of (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate (“Compound 2”).

### EXAMPLES

The present teachings include descriptions provided in the Examples that are not intended to limit the scope of any claim. The following non-limiting examples are provided to further illustrate the present teachings. Those of skill in the art, in light of the present application, will appreciate that many changes can be made in the specific embodiments that are provided herein and still obtain a like or similar result without departing from the spirit and scope of the present teachings.

### Abbreviations

ACN acetonitrile
AcOH acetic acid
δ chemical shift
DCM dichloromethane
DMAc N,N-dimethylacetamide
DMSO dimethylsulfoxide
EtOAc ethyl acetate
¹H NMR proton nuclear magnetic resonance
IPA isopropyl alcohol
IPAc isopropyl acetate
2-MeTHF 2-methyltetrahydrofuran
MEK methyl ethyl ketone
MIBK methyl isobutyl ketone
MTBE methyl tert-butyl ether
THF tetrahydrofuran

### Instrumentation and Methods

Unless otherwise indicated, the following instrumentation and methods were used in the working examples described herein.

### X-Ray Powder Diffraction (XRPD)

High resolution X-ray Powder Diffraction experiments were performed with Panalytical X'Pert³ Powder XRPD on a Si zero-background holder. The 20 position was calibrated against Panalytical 640 Si powder standard. Details of the XRPD method are listed in Table 1 below:

TABLE 1

| | Parameters for Reflection Mode |
|---|---|
| X-Ray wavelength | Cu, kα, Kα1 (Å): 1.540598, Kα2 (Å): 1.544426 |
| | Kα2/Kα1 intensity ratio: 0.50 |
| X-Ray tube setting | 45 kV, 40 mA |
| Divergence slit | Automatic |
| Scan mode | Continuous |
| Scan range (°2TH) | 3°-40° |
| Step size (°2TH) | 0.0131 |
| Scan speed (°/s) | 0.033 |

Peaks are reported as diffraction angles at 2 theta, with d-spacing measured in angstroms.

### Thermal Analysis

Thermo Gravimetric Analysis (TGA) experiments were performed on TA Q500 TGA from TA Instruments. Samples were heated at 10° C./min from about 20° C. to about 250° C. using dry nitrogen to purge the system. The details of the method are providing in Table 2, below:

TABLE 2

| Parameters | TGA |
|---|---|
| Pan Type | Platinum plate, open |
| Temperature | RT-250° C. |
| Ramp rate | 10° C./min |
| Purge gas | N₂ |

Differential Scanning Calorimetry (DSC) experiments were performed on TA Q2000 DSC from TA Instruments. Samples were heated at 10° C./min from about 20° C. to about 250° C. using dry nitrogen to purge the system. The details of the method are providing in Table 3, below:

TABLE 3

| Parameters | DSC |
|---|---|
| Pan Type | Aluminum pan, closed |
| Temperature | RT-250° C. |
| Ramp rate | 10° C./min |
| Purge gas | N₂ |

### Modulated Differential Scanning Calorimetry

Modulated Differential Scanning Calorimetry (mDSC) experiments were performed on TA Q2000 DSC from TA Instruments according to the following conditions reported in Table 4:

TABLE 4

| Parameters | Settings/Values |
|---|---|
| Method | Conventional MDSC |
| Temperature amplitude | 1.0° C. |
| Modulation | 60 s |
| Ramp rate | 3.0° C./min |
| Purge gas | N₂ at 50.00 mL/min |

### Dynamic Vapor Sorption

Dynamic Vapor Sorption (DVS) was obtained using a Surface Measurement Systems (SMS) DVS Intrinsic. The relative humidity at 25° C. were calibrated against deliquescence point of LiCl, Mg (NO₃)₂ and KCl. Typical parameters for DVS tests are listed in Table 5, below:

TABLE 5

| Parameters | Values |
|---|---|
| Temperature | 25° C. |
| Sample size | ~10-20 mg |
| Gas and flow rate | N₂, 200 mL/min |
| dm/dt | 0.002%/min |
| Min. dm/dt stability | 10 min |
| duration | |
| Max. equilibrium time | 180 min or 360 min |
| RH range | 20% RH-95% RH-0% RH-95% RH; |
| | 90% RH-0% RH-90% RH |
| RH step size | 10% (90% RH-0% RH-90% RH); |
| | 5% (95% RH-90% RH and 90% RH-95% RH) |

### High Pressure Liquid Chromatography

High Pressure Liquid Chromatography (HPLC) data were obtained according to Tables 6 or 7, below:

TABLE 6

| HPLC | Parameters | |
|---|---|---|
| Instrument | Agilent 1100 HPLC with DAD detector | |
| Column | Waters Sunfire C18, 4.6 × 150 mm, 5 μm | |
| Mobile Phase | A: 0.05% Trifluoroacetic acid (TFA) in water | |
| | B: 0.05% TFA in acetonitrile | |
| | Time (min) | B % |
| Gradient | 0.0 | 5 |
| | 2.0 | 20 |
| | 15 | 40 |
| | 21 | 90 |
| | 25 | 90 |
| | 28 | 5 |

| | |
|---|---|
| Flow Rate | 1.2 mL/min |
| Wave length | UV at 275 nm, UV at 254 nm |
| Injection Volume | 10 μL |
| Run time | 30 min |
| Post time | 0.0 min |
| Column Temperature | 35° C. |
| Sample temperature | RT |
| Diluent | 25% Acetonitrile in water |
| Target analytical | 0.25 mg/mL |
| concentration | |

TABLE 7

| HPLC | Parameters | |
|---|---|---|
| Instrument | Agilent 1100 HPLC with DAD detector | |
| Column | Kromasil, C¹⁸, 4.6*100 mm, 5 μm | |
| Wave length | 275 nm | |
| Injection volume | 10 μL | |
| Flow rate | 1.0 mL/min | |
| Column temp. | 35° C. | |
| Sampler temp. | RT | |
| Mobile phase | A: 0.1% H₃PO₄ in H₂O | |
| | B: 0.1% H₃PO₄ in ACN | |
| | Time (min) | B % |
| Gradient | 0 | 20 |
| | 10 | 30 |
| | 15 | 90 |
| | 18 | 90 |
| | 20 | 20 |

| | |
|---|---|
| Run Time | 20 min |

A standard stock was dissolved with diluent in a volumetric flask (e.g., 25 mg in 25 mL). This stock was vortexed and sonicated 10 sec to dissolve, and then diluted 1:10, 1:4, and 1:2 either in volumetric flasks or in HPLC vials to generate a standard curve. This standard curve had a minimum of 3 points and a minimum correlation of r²=0.999. Where appropriate, a blank was created to accurately replicate the matrix of the analytical sample, and peaks appearing in the blank were identified before integration and quantification using Chemstation software.

### Example 1—Synthesis of (S)-(5-cyclobutoxy-2-methyl-6-(1-(piperidin-4-yl)-1H-pyrazol-4-yl)-3,4-dihydroquinolin-1(2H)-yl)(cyclopropyl)methanone (Compound 1)

The synthesis of (S)-(5-cyclobutoxy-2-methyl-6-(1-(piperidin-4-yl)-1H-pyrazol-4-yl)-3,4-dihydroquinolin-1(2H)-yl)(cyclopropyl)methanone (“Compound 1”) was previously reported in PCT Application Publication No. WO 2015/074064, the entirety of which is incorporated by reference herein, and is repeated here.

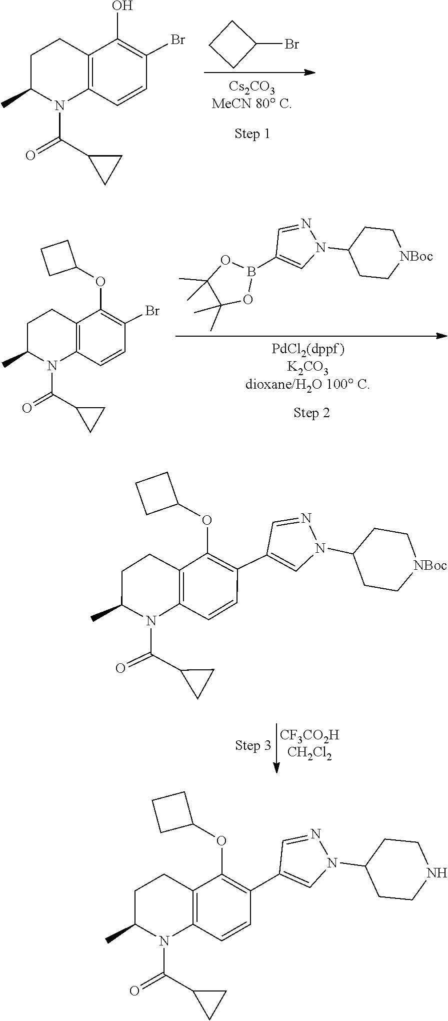

### Step 1—(2S)-6-bromo-5-cyclobutoxy-1-cyclopropanecarbonyl-2-methyl-1,2,3,4-tetrahydroquinoline

A 250-mL round-bottom flask was charged with (2S)-6-bromo-1-cyclopropanecarbonyl-2-methyl-1,2,3,4-tetrahydroquinolin-5-ol (2.00 g, 6.45 mmol), bromocyclobutane (1.81 mL, 2.60 g, 19.3 mmol), cesium carbonate (6.3 g, 19.34 mmol) and acetonitrile (100 mL). The resulting mixture was stirred for 6 h at 80° C. The reaction mixture was filtered through a pad of Celite and concentrated under vacuum. The residue was purified via column chromatography on silica gel (gradient elution with 0-10% ethyl acetate-petroleum ether) to afford (2S)-6-bromo-5-cyclobutoxy-1-cyclopropanecarbonyl-2-methyl-1,2,3,4-tetrahydroquinoline (2.00 g, 85%) as a colorless oil. MS (ES, m z): 364,366 [M+H]⁺.

### Step 2 (S)-tert-butyl 4-(4-(5-cyclobutoxy-1-(cyclopropanecarbonyl)-2-methyl-1,2,3,4-tetrahydroquinolin-6-yl)-1H-pyrazol-1-yl)piperidine-1-carboxylate

A 250-mL round-bottom flask was purged and maintained with an inert atmosphere of nitrogen, and charged with (2S)-6-bromo-5-cyclobutoxy-1-cyclopropanecarbonyl-2-methyl-1,2,3,4-tetrahydroquinoline (2.0 g, 5.5 mmol), tert-butyl 4-[4-(tetramethyl-1,3,2-dioxaborolan-2-yl)-1H-pyrazol-1-yl]piperidine-1-carboxylate (2.5 g, 6.63 mmol), [1,1′-bis(diphenylphosphino)ferrocene]dichloropalladium(II) dichloromethane adduct (0.45 g, 0.55 mmol), potassium carbonate (2.3 g, 16.64 mmol), 1,4-dioxane (50 mL) and water (5 mL). The resulting mixture stirred overnight at 100° C. The reaction mixture was cooled to room temperature and then filtered through a pad of Celite. The filtrate was concentrated, and the residue was purified via column chromatography on silica gel (gradient elution with 0-30% ethyl acetate-petroleum ether) to afford (S)-tert-butyl 4-(4-(5-cyclobutoxy-1-(cyclopropanecarbonyl)-2-methyl-1,2,3,4-tetrahydroquinolin-6-yl)-1H-pyrazol-1-yl)piperidine-1-carboxylate (2.2 g, 75%) as a light yellow solid. MS (ES, m z): 535 [M+H]⁺.

### Step 3 (2S)-5-cyclobutoxy-1-cyclopropanecarbonyl-2-methyl-6-[1-(piperidin-4-yl)-1H-pyrazol-4-yl]-1,2,3,4-tetrahydroquinoline

Trifluoroacetic acid (0.5 mL, 6.49 mmol) was added to a 0° C. solution of (S)-tert-butyl 4-(4-(5-cyclobutoxy-1-(cyclopropanecarbonyl)-2-methyl-1,2,3,4-tetrahydroquinolin-6-yl)-1H-pyrazol-1-yl)piperidine-1-carboxylate (0.032 g, 0.060 mmol) in dichloromethane (2.0 mL). The ice bath was removed, and the mixture stirred at rt for 1.5 h. The reaction mixture was concentrated, and the residue was partitioned between dichloromethane and saturated aqueous sodium bicarbonate solution. The layers were separated and the organic phase was washed with brine, dried over anhydrous sodium sulfate, filtered, and concentrated to afford (S)-(5-cyclobutoxy-2-methyl-6-(1-(piperidin-4-yl)-1H-pyrazol-4-yl)-3,4-dihydroquinolin-1(2H)-yl)(cyclopropyl)methanone (Compound 1) (0.022 g, 85%) as an off-white solid. ¹H NMR (300 MHz, CHLOROFORM-d) 6 ppm: 0.57-0.71 (m, 1H), 0.78-0.91 (m, 1H), 0.92-1.04 (m, 1H), 1.13 (d, J=6.45 Hz, 3H), 1.19-1.45 (m, 3H), 1.53-1.71 (m, 2H), 1.79-2.42 (m, 11H), 2.74-2.92 (m, 2H), 2.94-3.09 (m, 1H), 3.30 (br d, J=12.61 Hz, 2H), 4.04-4.21 (m, 1H), 4.28 (ddt, J=11.43, 7.62, 3.96, 3.96 Hz, 1H), 4.66-4.84 (m, 1H), 7.12 (d, J=8.21 Hz, 1H), 7.28 (d, J=8.21 Hz, 1H), 7.81 (s, 1H), 7.88 (s, 1H). MS (ESI, pos. ion) m/z 435 [M+H]⁺.

### Example 2—Salt Screening of Compound 1

In an effort to find salts of Compound 1, a salt/co-crystal screen was conducted under 127 different conditions with 25 salt/co-crystal co-formers and five solvent systems. For each condition, about 20 mg of Compound 1 was dispersed in the selected solvent in a glass vial, and then salt/co-crystal co-former was added. After sonication and stirring at room temperature (RT), resulting solids were isolated and analyzed by XRPD. The results are summarized in Table 8, below:

TABLE 8

| Acids, molar ratio | | | | MeOH/H₂O | |
|---|---|---|---|---|---|
| (Compound 1:acid) | ACN | Acetone | EtOAc | (19:1, v/v) | 1,4-Dioxane |
| Hydrochloric acid, (1:1) | E | E | E | E | E |
| Hydrochloric acid, (1:2) | N/A | E | E | E | E |
| Sulfuric acid, (1:0.5) | N/A | N/A | N/A | Amorphous⁺ | E |
| Sulfuric acid, (1:1) | N/A | N/A | N/A | E | N/A |
| Phosphoric acid, (1:1) | Amorphous* | E | Amorphous* | Amorphous⁺ | N/A |
| Maleic Acid, (1:1) | E | E | Maleate | E | E |
| | | | Form A | | |
| Nicotinic Acid, (1:1) | E | E | E | E | E |
| Citric Acid, (1:1) | N/A | N/A | N/A | E | E |
| Glycolic Acid/hydroxyacetic | N/A | E | N/A | E | E |
| acid, (1:1) | | | | | |
| Fumaric Acid, (1:1) | Fumarate | Fumarate | Fumarate | E | Amorphous |
| | Form A | Form B | Form C | | |
| L-Tartaric Acid, (1:1) | N/A | N/A | N/A | E | N/A |
| (R)-(−)-Mandelic acid, (1:1) | E | E | E | E | E |
| Hippuric Acid, (1:1) | N/A | E | E | E | E |
| Succinic Acid, (1:1) | N/A | Succinate | N/A | E | E |
| | | Form A* | | | |
| Glutaric Acid, (1:1) | N/A | N/A | N/A | E | E |
| Adipic Acid, (1:1) | N/A | N/A | N/A | E | Adipate |
| | | | | | Form A* |
| Acetic acid, (1:1) | E | E | E | E | E |
| 2,5-Dihydroxybenzoic, (1:1) | E | E | N/A | E | E |
| Methansulfonic acid, (1:1) | E | E | E | E | E |
| Malonic acid, (1:1) | E | E | N/A | E | E |
| benzensulfonic acid, (1:1) | E | E | E | E | E |
| Nicotinamide, (1:1) | Nicotinamide* | Nicotinamide⁺ | Nicotinamide* | E | E |
| 1,2-Ethanedisulfonic | N/A | N/A | N/A | E | N/A |
| acid dihydrate, (1:1) | | | | | |
| Mucic acid, (1:1) | Mucic acid + | Mucic acid + | Mucic acid | Mucic acid + | Mucic acid + |
| | new peaks | new peaks | | new peaks | new peaks |
| L-Glutamide, (1:1) | L-Glutamide | L-Glutamide | L-Glutamide | L-Glutamide | L-Glutamide |

| | |
|---|---|
| NaOH, (1:1) | E (MeOH/H₂O, 3/1 v/v as solvent) |
| KOH, (1:1) | E (MeOH/H₂O, 3/1 v/v as solvent) |

N/A: Jell or oil obtained after stirring
E: Jell or oil obtained after evaporation at RT
*Solid obtained from slurry at 4° C.
⁺Solid obtained from evaporation

A total of six crystalline salt/co-crystals were identified: Compound 1 Fumarate Form A, Compound 1 Fumarate Form B, Compound 1 Fumarate Form C, Compound 1 Succinate Form A, Compound 1 Adipate, Form A, and Compound 1 Maleate Form A.

### Example 3—Characterization of Salt Forms of Compound 1 Compound 1 Fumarate Form A

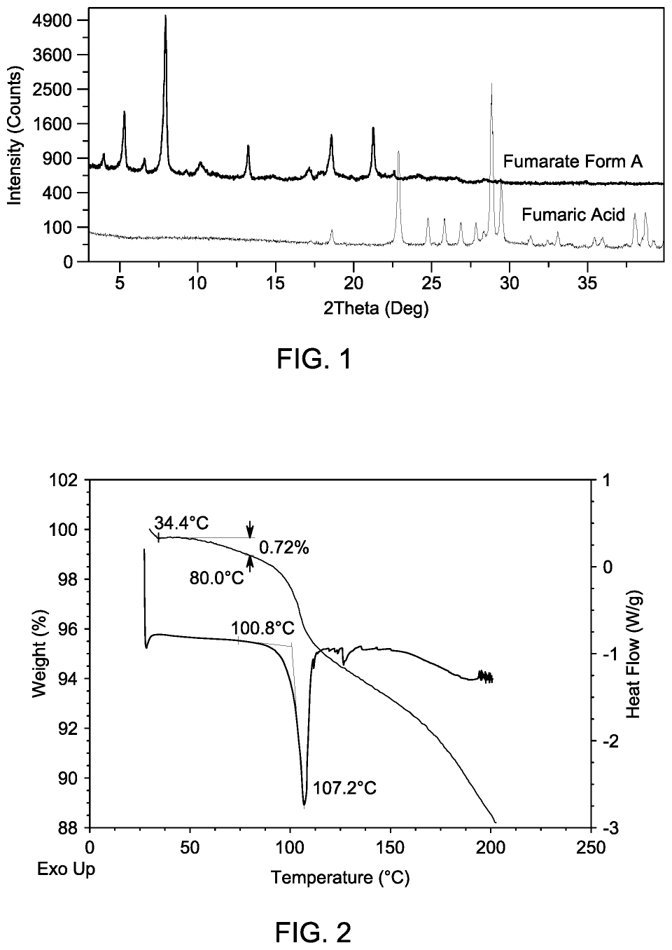

Compound 1 Fumarate Form A (“Fumarate Form A”) was obtained from a slurry of fumaric acid and Compound 1 (1:1 molar ratio) in ACN. The solid was isolated and characterized by XRPD, TGA, and DSC. As shown by the XRPD pattern in FIG. 1, Fumarate Form A is crystalline and has a distinguished pattern from that of fumaric acid. The XRPD pattern of the crystalline Compound 1 Fumarate Form A is depicted in FIG. 1, and the corresponding data is summarized below:

| 2 theta | d-spacing |
|---|---|
| 4.0 | 22.1 |
| 5.3 | 16.7 |
| 6.6 | 13.4 |
| 7.9 | 11.1 |
| 10.2 | 8.7 |
| 13.3 | 6.7 |
| 14.8 | 6.0 |
| 17.2 | 5.2 |
| 18.6 | 4.8 |
| 21.2 | 4.2 |
| 24.1 | 3.7 |
| 26.5 | 3.4 |
| 28.4 | 3.1 |

As shown by TGA and DSC curves in FIG. 2, the sample had 0.7% weight loss before 80° C. followed by continuous weight loss and an endothermic peak at 107.2° C. (peak temperature).

### Compound 1 Fumarate Form B

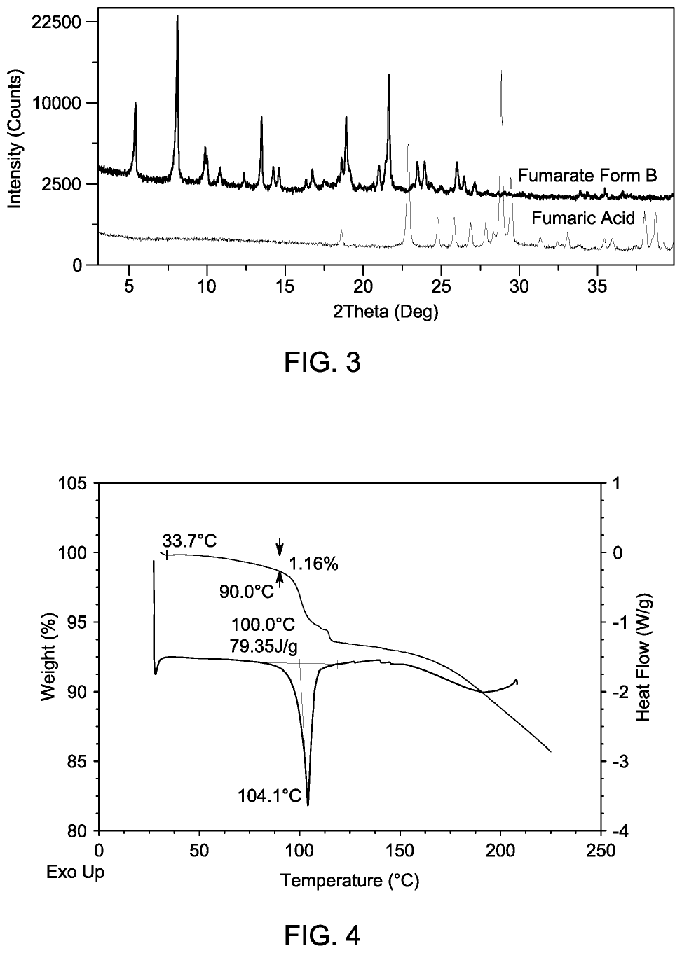

Compound 1 Fumarate Form B (“Fumarate Form B”) was observed from slurry of fumaric acid and Compound 1 (1:1 molar ratio) in acetone. The solid was isolated and characterized by XRPD, TGA, and DSC. As shown by the XRPD pattern in FIG. 3, Fumarate Form B is crystalline and has a distinguished pattern from that of fumaric acid. The XRPD pattern of the crystalline Compound 1 Fumarate Form B is depicted in FIG. 3, and the corresponding data is summarized below:

| 2 theta | d-spacing |
|---|---|
| 5.4 | 16.4 |
| 8.1 | 10.9 |
| 9.9 | 9.0 |
| 10.0 | 8.8 |
| 10.9 | 8.1 |
| 12.4 | 7.2 |
| 13.5 | 6.6 |
| 14.2 | 6.2 |
| 14.6 | 6.1 |
| 16.3 | 5.4 |
| 16.8 | 5.3 |
| 17.5 | 5.1 |
| 18.6 | 4.8 |
| 18.9 | 4.7 |
| 21.1 | 4.2 |
| 21.6 | 4.1 |
| 23.5 | 3.8 |
| 23.9 | 3.7 |
| 26.0 | 3.4 |
| 26.5 | 3.4 |
| 27.1 | 3.3 |
| 34.1 | 2.6 |
| 35.5 | 2.5 |
| 36.8 | 2.4 |

As shown by TGA and DSC curves in FIG. 4, Fumarate Form B had 1.2% weight loss before 90° C. followed by continuous weight loss and an endothermic peak at 104.1° C. (peak temperature).

### Compound 1 Fumarate Form C

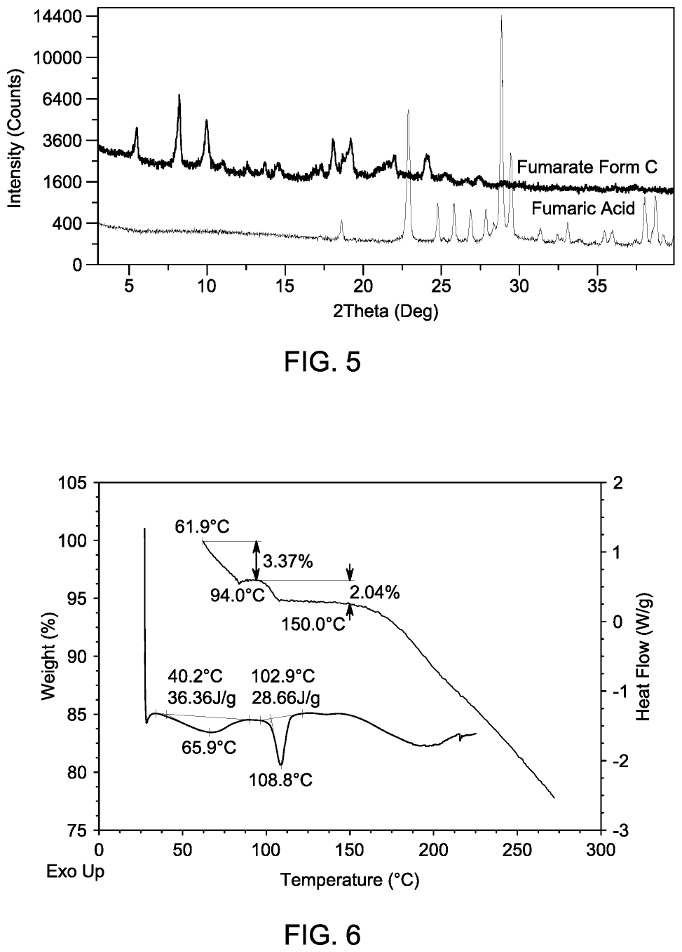

Compound 1 Fumarate Form C (“Fumarate Form C”) was observed from slurry of fumaric acid and Compound 1 (1:1 molar ratio) in EtOAc. The solid was isolated and characterized by XRPD, TGA, and DSC. As shown by the XRPD pattern in FIG. 5, Fumarate Form C is crystalline and has a distinguished pattern from that of fumaric acid. The XRPD pattern of the crystalline Compound 1 Fumarate Form C is depicted in FIG. 5, and the corresponding data is summarized below:

| 2 theta | d-spacing |
|---|---|
| 5.5 | 16.1 |
| 8.2 | 10.7 |
| 10.0 | 8.9 |
| 13.7 | 6.5 |
| 14.6 | 6.1 |
| 18.1 | 4.9 |
| 19.2 | 4.6 |
| 22.0 | 4.0 |
| 24.1 | 3.7 |
| 25.3 | 3.5 |
| 27.4 | 3.3 |

As shown by TGA curve in FIG. 6, Fumarate Form C showed stepwise weight loss of 3.4% before 94° C. and 2.0% from 94° C. to 150° C., followed by continuous weight loss. Two endothermic peaks at 65.9° C. and 108.8° C. (peak temperature) were observed in DSC curve shown in FIG. 6.

### Compound 1 Adipate Form A

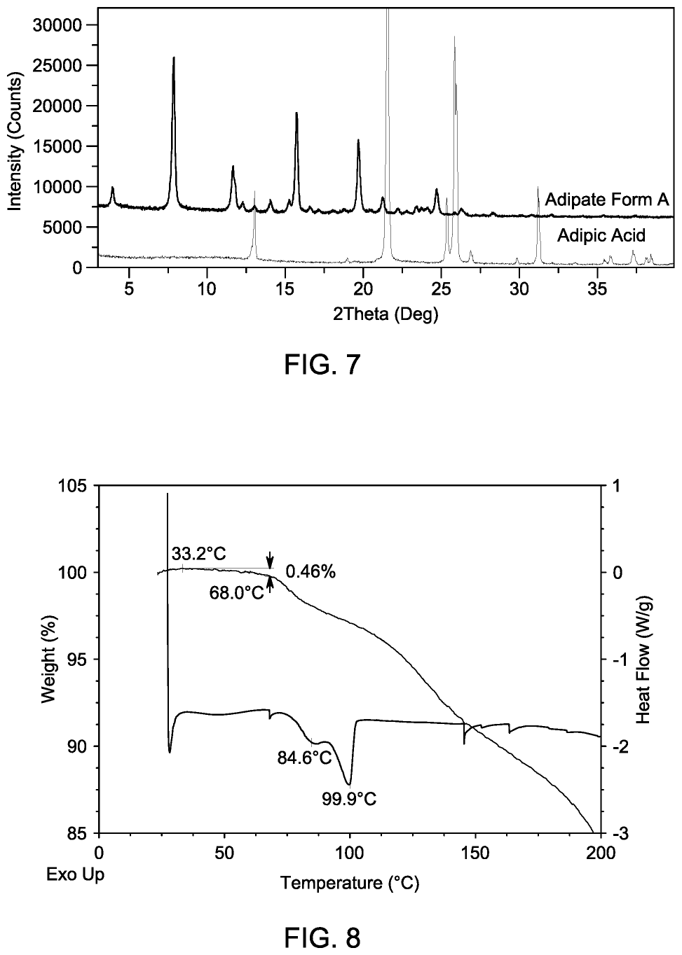

Compound 1 Adipate Form A (“Adipate Form A”) was observed from slurry of adipic acid and Compound 1 (1:1 molar ratio) in 1,4-dioxane. The solid was isolated and characterized by XRPD, TGA, and DSC. As shown by the XRPD result in FIG. 7, Adipate Form A is crystalline and has a distinguished pattern from that of adipic acid. The XRPD pattern of the crystalline Compound 1 Adipate Form A is depicted in FIG. 7, and the corresponding data is summarized below:

| 2 theta | d-spacing |
|---|---|
| 4.0 | 22.3 |
| 7.9 | 11.2 |
| 11.6 | 7.6 |
| 12.3 | 7.2 |
| 13.1 | 6.8 |
| 14.1 | 6.3 |
| 15.3 | 5.8 |
| 15.7 | 5.6 |
| 16.6 | 5.3 |
| 18.8 | 4.7 |
| 19.7 | 4.5 |
| 21.2 | 4.2 |
| 22.2 | 4.0 |
| 23.4 | 3.8 |
| 24.1 | 3.7 |
| 24.7 | 3.6 |
| 26.3 | 3.4 |
| 28.3 | 3.2 |
| 30.8 | 2.9 |

As shown by TGA and DSC curves in FIG. 8, Adipate Form A had 0.5% weight loss before 68° C., followed by continuous weight loss and two endothermic peaks at 84.6° C. and 99.9° C. (peak temperature).

### Compound 1 Succinate Form A (i.e., Compound 2 Form A)

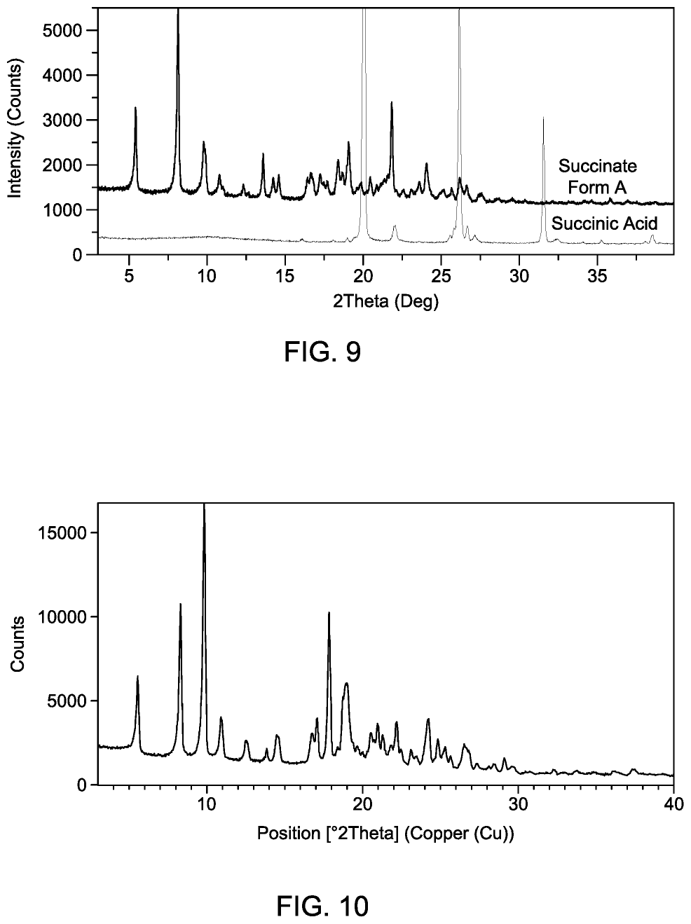

Compound 1 Succinate Form A (i.e., Compound 2 Form A, “Succinate Form A”) was observed from slurry of succinic acid and Compound 1 (1:1 molar ratio) in acetone. The solid was isolated at 4° C. and characterized by XRPD, TGA, and DSC. As shown by the XRPD result in FIG. 9, Succinate Form A is crystalline and has a distinguished pattern from that of succinic acid. The XRPD pattern of the crystalline Compound 1 Succinate Form A, i.e., Compound 2 Form A, is depicted in FIG. 9 and FIG. 10, and the corresponding data is summarized below:

| 2 theta | d-spacing |
|---|---|
| 5.5 | 16.0 |
| 8.3 | 10.7 |
| 9.8 | 9.0 |
| 10.8 | 8.2 |
| 12.5 | 7.1 |
| 12.6 | 7.0 |
| 13.8 | 6.4 |
| 14.4 | 6.1 |
| 14.6 | 6.1 |
| 16.7 | 5.3 |
| 17.1 | 5.2 |
| 17.9 | 5.0 |
| 18.4 | 4.8 |
| 18.7 | 4.7 |
| 19.0 | 4.7 |
| 19.6 | 4.5 |
| 20.0 | 4.4 |
| 20.5 | 4.3 |
| 20.9 | 4.2 |
| 21.3 | 4.2 |
| 21.8 | 4.1 |
| 22.2 | 4.0 |
| 22.5 | 4.0 |
| 23.1 | 3.8 |
| 23.4 | 3.8 |
| 24.2 | 3.7 |
| 24.8 | 3.6 |
| 25.3 | 3.5 |
| 25.6 | 3.5 |
| 26.5 | 3.4 |
| 26.8 | 3.3 |
| 27.3 | 3.3 |
| 28.1 | 3.2 |
| 28.5 | 3.1 |
| 29.1 | 3.1 |
| 29.7 | 3.0 |
| 30.7 | 2.9 |
| 32.3 | 2.8 |
| 32.9 | 2.7 |
| 33.8 | 2.7 |
| 34.9 | 2.6 |
| 36.1 | 2.5 |
| 37.3 | 2.4 |

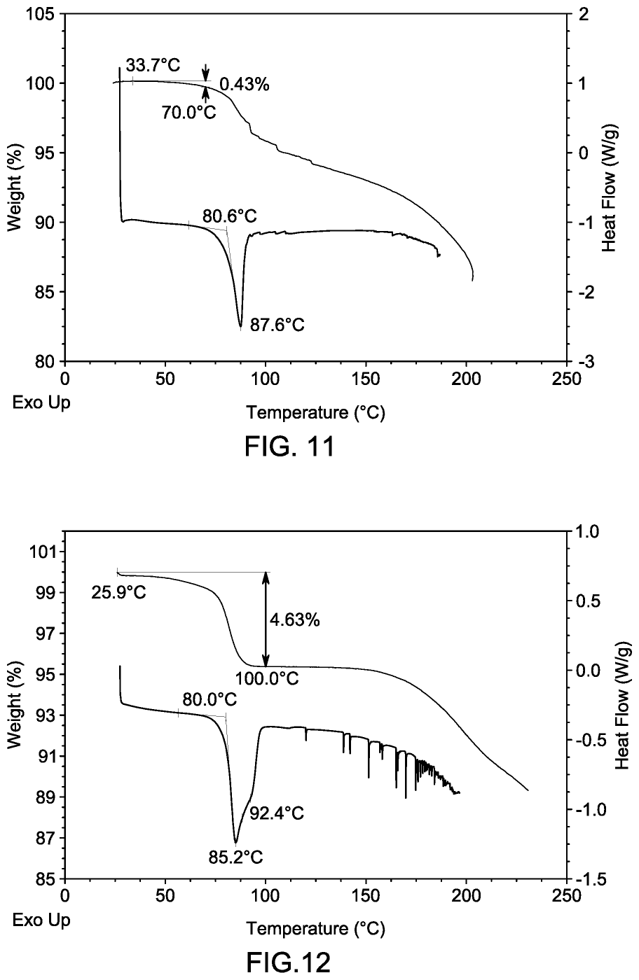

As shown by TGA and DSC curves in FIG. 11, Succinate Form A had 0.4% weight loss before 70° C., followed by continuous weight loss and an endothermic peak at 87.6° C. (peak temperature).

As shown by TGA and DSC curves in FIG. 12, Succinate Form A showed a weight loss of 5.1% up to 100° C. and one endotherm at 84.0° C. (onset temperature) before decomposition.

The chemical stability of Succinate Form A was determined using HPLC-UV at 275 nm and 254 nm.

As seen in Table 9, Succinate Form A showed no significant chemical degradation after 42 days at temperatures below 40° C., and did not show significant chemical degradation after 42 days at 25° C./60% RH.

TABLE 9

| | | | | | % Purity (254 nm) | | | | | | |
|---|---|---|---|---|---|---|---|---|---|---|---|
| | | | | | Retention Time (min) | | | | | | |
| | | | | | 4.5 | 5.3 | 6.9 | 7.5 | 8.0 | 9.3 | 11.1 |
| | Succinate | | % Claim | % Purity | Relative Retention Time | | | | | | |
| Ex. No. | Form | Condition | (275 nm) | (275 nm) | 0.36 | 0.42 | 0.55 | 0.59 | 0.64 | 0.74 | 0.89 |
| 1 | A | initial | 95.2% | 98.73% | | 0.30 | 0.78 | 1.03 | | | |
| 2 | A | 25° C./60% RH | 99.8% | 98.78% | | 0.26 | 0.79 | 0.84 | | | |
| 3 | A | 25° C./60% RH | 98.3% | 98.64% | 0.08 | 0.08 | 0.79 | 0.86 | | | |
| 4 | G | initial | 102% | 98.84% | | 0.20 | 0.62 | 0.93 | | | |
| 5 | G | RT | 106% | 98.85% | | 0.20 | 0.63 | 0.86 | | | |
| 6 | G | RT | 103% | 98.88% | | 0.18 | 0.62 | 0.85 | | | |
| 7 | G | RT | 106% | 98.82% | 0.04 | 0.18 | 0.63 | 0.90 | | | |
| 8 | G | RT | 107% | 98.86% | 0.04 | 0.15 | 0.63 | 0.90 | | | |
| 9 | G | 25° C./60% RH | 104% | 98.84% | | 0.19 | 0.63 | 0.87 | | | |
| 10 | G | 25° C./60% RH | 106% | 98.84% | | 0.18 | 0.62 | 0.84 | | | |
| 11 | G | 25° C./60% RH | 107% | 98.84% | 0.05 | 0.14 | 0.63 | 0.88 | | | |
| 12 | G | 25° C./60% RH | 103% | 98.86% | 0.04 | 0.12 | 0.62 | 0.89 | | | |
| 13 | G | 40° C./75% RH | 106% | 98.51% | 0.12 | | 0.62 | 0.88 | | | |
| 14 | G | 40° C./75% RH | 106% | 98.33% | 0.12 | | 0.61 | 0.87 | | 0.05 | |
| 15 | G | 40° C./75% RH | 96.3% | 97.37% | 0.12 | | 0.63 | 0.83 | 0.05 | 0.08 | |
| 16 | G | 40° C./75% RH | 101% | 96.72% | 0.13 | | 0.62 | 0.84 | 0.05 | 0.07 | |
| 17 | G | 50° C. | 100% | 98.69% | | 0.19 | 0.62 | 0.83 | | | |
| 18 | G | 50° C. | 101% | 97.37% | 0.05 | 0.14 | 0.61 | 0.79 | | | |
| 19 | G | 50° C. | 104% | 97.79% | 0.05 | 0.15 | 0.61 | 0.78 | | | 0.06 |
| 20 | G | 50° C.* | 98.1% | 94.24% | 0.07 | 0.64 | 0.54 | 0.55 | | | 0.14 |
| 21 | G | Post DVS | 73.5% | 98.91% | | 0.20 | 0.62 | 0.97 | | | |

| | % Purity (254 nm) | | | | | | | | | |
|---|---|---|---|---|---|---|---|---|---|---|
| | Retention Time (min) | | | | | | | | | |
| | 11.5 | 12.2 | 12.3 | 12.6 | 14.2 | 14.5 | 15.8 | 17.4 | 19.2 | 20.1 |
| | Relative Retention Time | | | | | | | | | |
| Ex. No. | 0.91 | 0.97 | 0.98 | 1.00 | 1.13 | 1.15 | 1.26 | 1.38 | 1.52 | 1.59 |
| 1 | | | 0.47 | 96.98 | | 0.30 | 0.04 | | | 0.10 |
| 2 | | | 0.47 | 97.35 | | 0.30 | | | | |
| 3 | | | 0.47 | 97.24 | | 0.30 | 0.05 | | 0.13 | |
| 4 | | | 0.45 | 97.47 | | 0.21 | 0.07 | | | 0.05 |
| 5 | | | 0.45 | 97.58 | | 0.21 | | | 0.07 | |
| 6 | | | 0.46 | 97.60 | | 0.21 | | | | 0.08 |
| 7 | | | 0.45 | 97.51 | | 0.21 | | | 0.08 | |
| 8 | | | 0.45 | 97.55 | | 0.21 | | | 0.07 | |
| 9 | | | 0.45 | 97.52 | | 0.21 | | | 0.07 | 0.07 |
| 10 | | | 0.46 | 97.57 | | 0.21 | | | 0.08 | 0.04 |
| 11 | | | 0.45 | 97.56 | | 0.21 | | | 0.09 | |
| 12 | | | 0.44 | 97.59 | | 0.21 | | | 0.08 | |
| 13 | 0.06 | | 0.45 | 97.37 | | 0.21 | | | 0.25 | 0.05 |
| 14 | 0.06 | | 0.45 | 97.21 | | 0.21 | | | 0.42 | |
| 15 | 0.08 | | 0.44 | 96.31 | | 0.21 | | | 1.08 | 0.16 |
| 16 | 0.06 | | 0.45 | 95.81 | | 0.20 | | | 1.77 | |
| 17 | | | 0.45 | 97.17 | | 0.21 | | | 0.14 | 0.39 |
| 18 | | | 0.48 | 96.25 | | 0.21 | | | 1.47 | |
| 19 | | | 0.49 | 96.24 | | 0.21 | | | 0.90 | 0.52 |
| 20 | | 0.10 | 0.44 | 93.41 | 0.06 | 0.18 | | 0.05 | 4.40 | |
| 21 | | | 0.45 | 97.47 | | 0.21 | | | | 0.07 |

### Compound 1 Maleate Form A

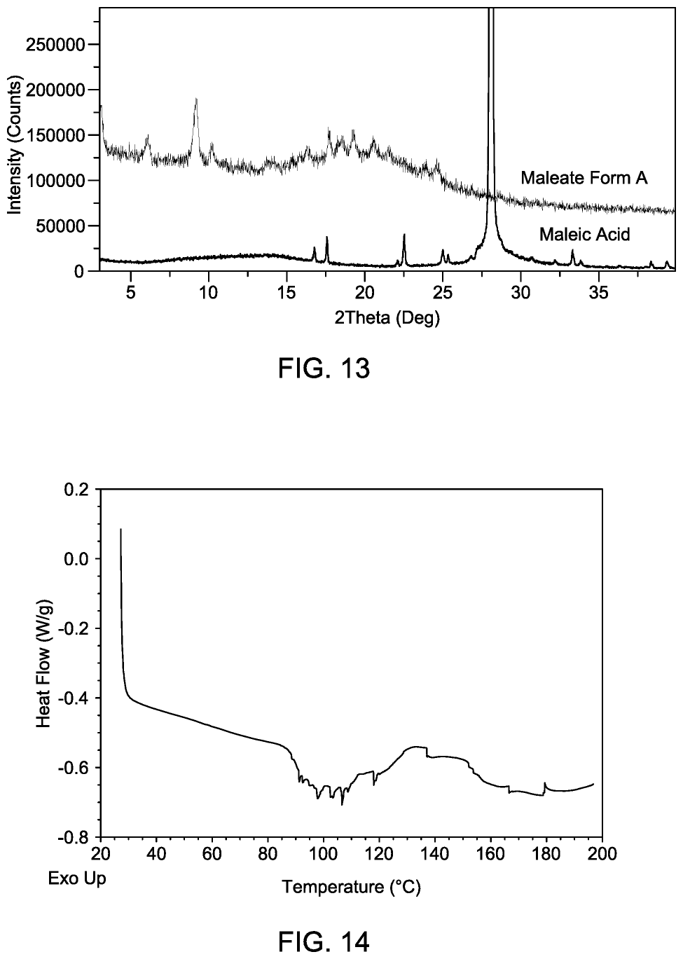

Compound 1 Maleate Form A (“Maleate Form A”) was observed from slurry of maleic acid and Compound 1 (1:1 molar ratio) in EtOAc. The solid was isolated at 4° C. and characterized by XRPD and DSC. As shown by the XRPD pattern in FIG. 13, Maleate Form A has a distinguished pattern from that of maleic acid. The XRPD pattern of the crystalline Compound 1 Maleate Form A is depicted in FIG. 13, and the corresponding data is summarized below:

| 2 theta | d-spacing |
|---|---|
| 3.1 | 28.6 |
| 6.1 | 14.5 |
| 9.2 | 9.6 |
| 10.2 | 8.6 |
| 13.9 | 6.4 |
| 16.3 | 5.5 |
| 17.7 | 5.0 |
| 18.5 | 4.8 |
| 19.3 | 4.6 |
| 20.6 | 4.3 |
| 21.5 | 4.1 |
| 24.5 | 3.6 |

The DSC curve of Maleate Form A is provided in FIG. 14.

### Example 4—Hygroscopicity Evaluation of Compound 1 Salt Forms

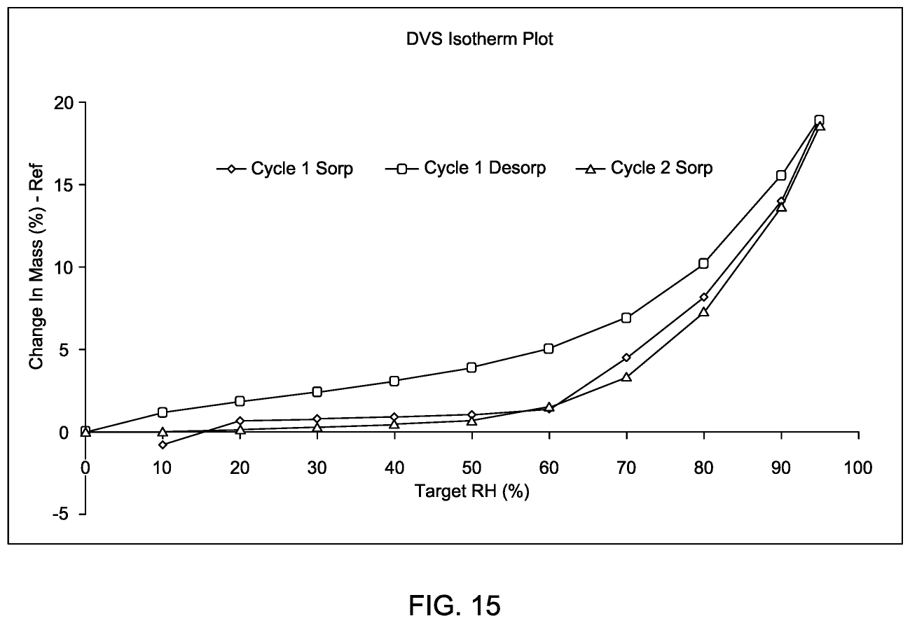

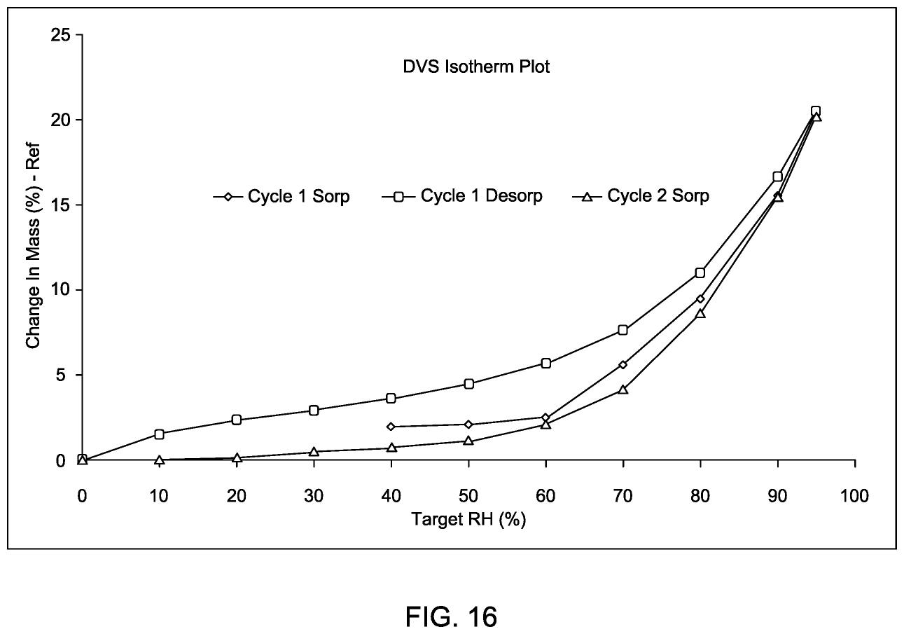

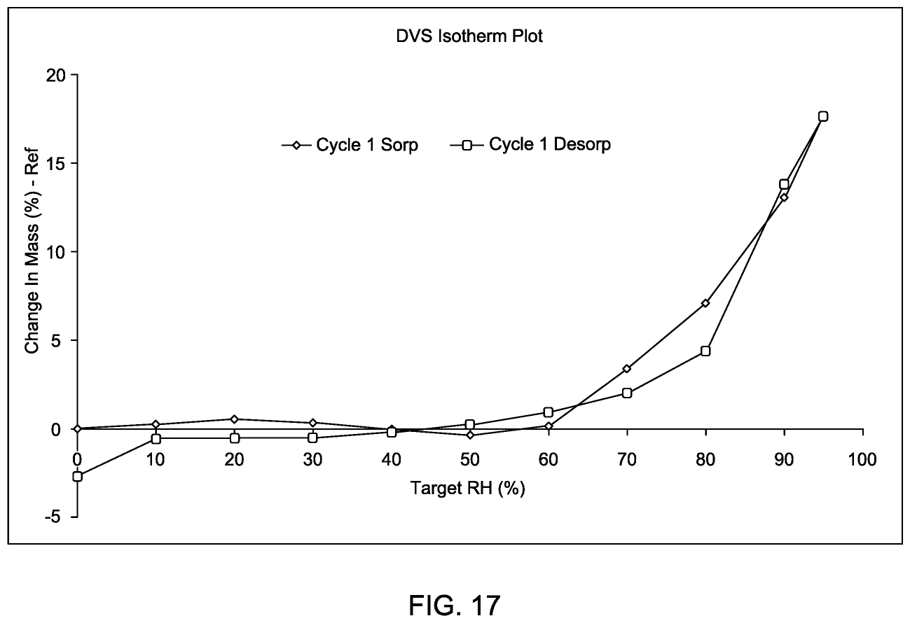

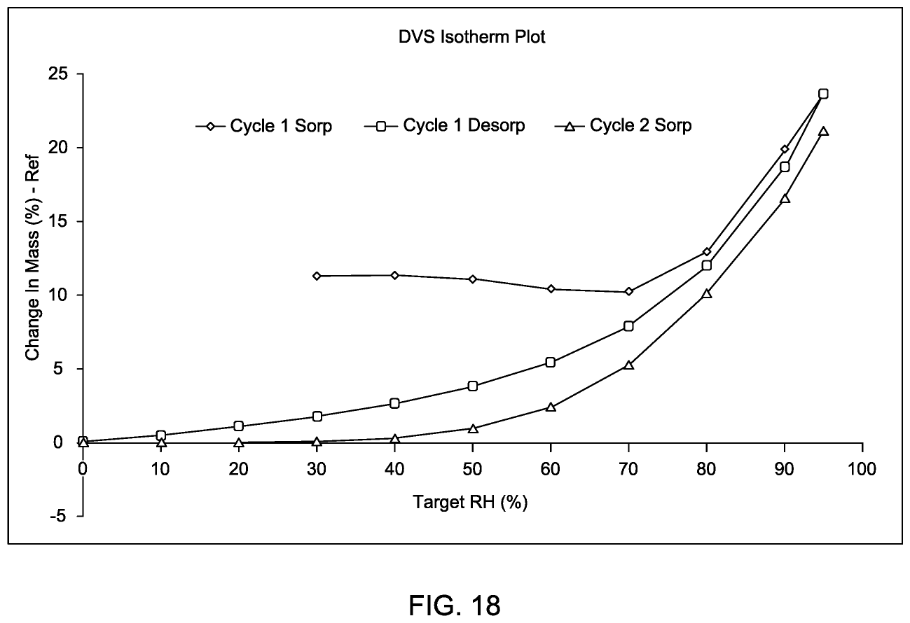

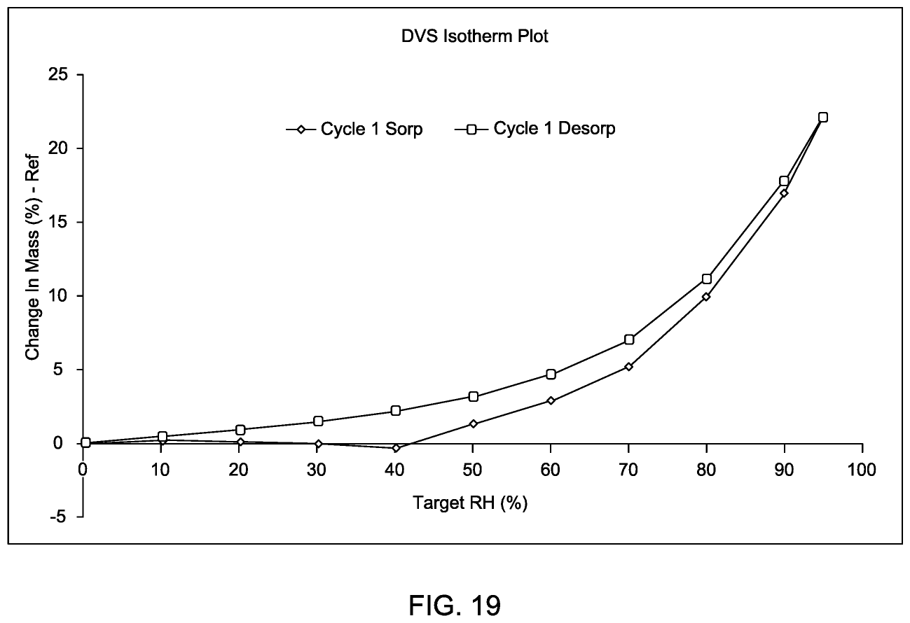

Hygroscopicity of five solid forms of Compound 1 salts (Fumarate Form A, Fumarate Form B, Fumarate Form C, Adipate Form A, and Succinate Form A) was evaluated by DVS and XRPD analysis (for the solids after DVS). The results are shown in FIGS. 15-19. Based on current data, all five solid forms became deliquescent at high humidity (>60% RH), resulting in amorphous or jelly solids after the DVS test.

### Example 5—Aqueous Solubility Evaluation of Compound 1 Salt Forms

Aqueous solubility of five solid forms of Compound 1 salts (Fumarate Form A, Fumarate Form B, Fumarate Form C, Adipate Form A, and Succinate Form A) was evaluated. For each of the experiments 1 through 5 in Table 10, about 100 mg of solid form was suspended in 1.5 mL of water, and the vial was rolled at RT for about 24 hours (25 r/min). Clear solutions were observed after rolling for three hours for all the samples except experiment 3 (Fumarate Form C). The solutions remained clear after 24 hours of rolling at RT. Supernatant of experiment 3 was collected by filtration through a 0.45 μm membrane for HPLC analysis. For each of the experiments 6 through 10 in Table 10, about 40 mg of solid form was dissolved in 0.2 mL (0.9 mL for experiment 7) water and then analyzed by HPLC after 24-hour storage at RT. As shown in Table 10, below, Fumarate Form A, Fumarate Form B, Adipate Form A and Succinate Form A indicated relatively high solubility (>100 mg FB/mL) in water while Fumarate Form C showed lower solubility (˜25-50 mg FB/mL) compared with others.

TABLE 10

| | | | Solubility, |
|---|---|---|---|
| Exp. ID | Method | Starting Salt Form | mg FB/mL |
| 1 | ~100 mg of salt | Fumarate Form A | Clear |
| 2 | in 1.5 mL water | Fumarate Form B | Clear |
| 3 | | Fumarate Form C | 45.3* |
| 4 | | Adipate Form A | Clear |
| 5 | | Succinate Form A | Clear |
| 6 | ~40 mg of salt in | Fumarate Form A | >127.9** |
| 7 | 0.2 mL (0.9 mL for | Fumarate Form B | >102.3** |
| 8 | Fumarate Form C) | Fumarate Form C | >25.8** |
| 9 | water | Adipate Form A | >108.9** |
| 10 | | Succinate Form A | >108.7** |

*Identity of remaining solids unknown due to sample scarcity
**Clear and jelly solution with no obvious solid particles

### Example 6—Slurry Conversions of Certain Compound 1 Salt Forms

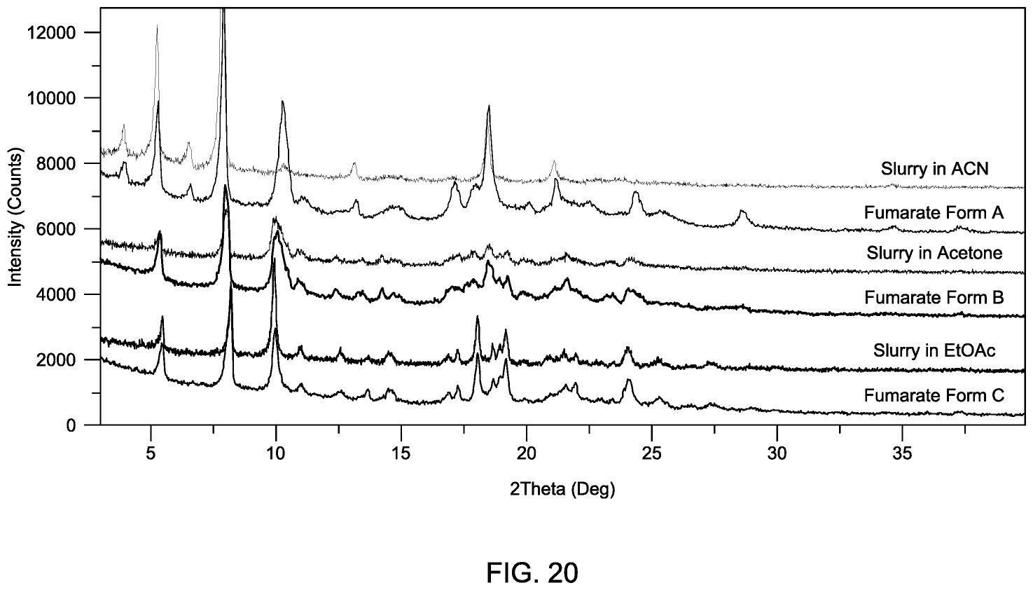

Slurry conversion of Fumarate Forms A, B, and C was conducted in three different solvents. A mixture of Fumarate Form A/B/C (mass ratio 1:1:1) was stirred in ACN, acetone and EtOAc at RT for about 4 days. As shown by the results (Table 11 and FIG. 20), three fumarate salt forms were obtained respectively in correspondent solvents, suggesting the solvated states of Fumarate A, B, and C.

TABLE 11

| Starting material | Solvent | Solid form |
|---|---|---|
| Fumarate Type A, B and C | ACN | Fumarate Form A |
| (mass ratio: 1:1:1) | Acetone | Fumarate Form B |
| | EtOAc | Fumarate Form C |

### Example 7—Recrystallization of Adipate Form A

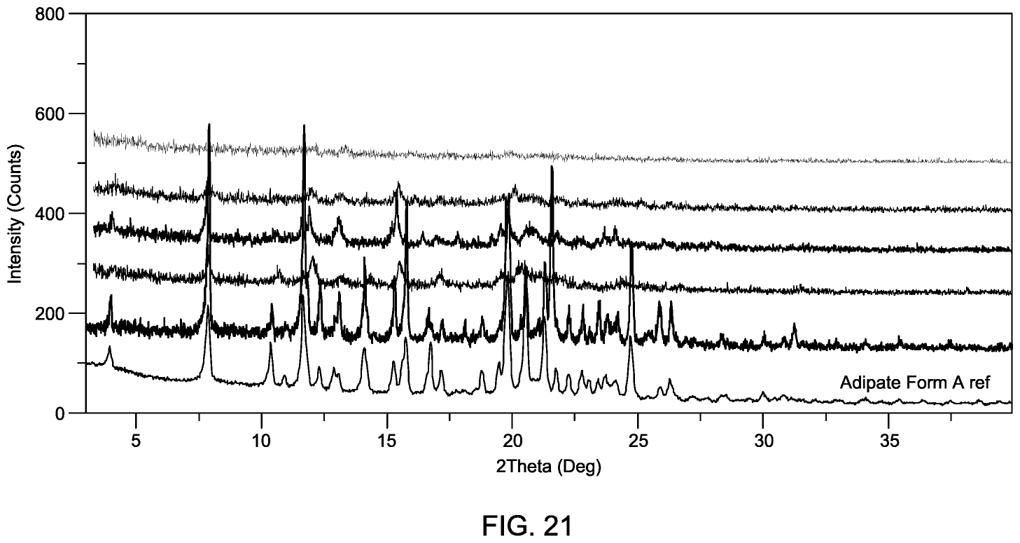

Recrystallization of adipate was conducted in several solvent systems with or without Adipate Form A seeds. A mixture of Compound 1 and adipic acid (molar ratio 1:1) was stirred with or without Adipate Form A at RT overnight. Remaining solids were isolated for XRPD analysis. As shown in Table 12 and FIG. 21, no new crystalline form was observed (Adipate Form A labeled, remaining series not labeled because no forms were observed).

TABLE 12

| Exp. ID | Method | Solvent, v/v | Solid Form |
|---|---|---|---|
| 1 | Without | Acetone | N/A |
| 2 | seeds | EtOAc/1,4-dioxane, | Adipate Form A + |
| | | 3:1 | acid |
| 3 | | EtOH | N/A |
| 4 | | IPA | N/A |
| 5 | With seeds | EtOH | N/A |
| 6 | of adipate | IPAc | Poor crystallinity |
| 7 | Form A | MIBK | Poor crystallinity |
| 8 | | MTBE | Amorphous |
| 9 | | Toluene | Poor crystallinity |

N/A: Clear solution resulted after stirring at RT overnight

### Example 8—Polymorph Screening of Compound 2

Polymorph screening for Compound 2 ((S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate) was conducted under at least 100 different conditions, including anti-solvent addition, evaporation, slurry, solid/liquid vapor diffusion, crash cooling and grinding. A summary of the experiments performed is reported in Table 13:

TABLE 13

| | No. of | |
|---|---|---|
| Method | Experiments | Isolated Crystal Forms |
| Anti-solvent addition | 22 | Form A, C, B, D, G |
| Slow evaporation | 13 | Form F, J |
| Crash cooling | 12 | Form A, C, D, K |
| Slurry at RT | 12 | Form A, B, G, J, L |
| Slurry at 50° C. | 12 | Form A, B, G, I, J |
| Solid vapor diffusion | 13 | Form A, C, D, E |
| Solution vapor diffusion | 14 | Form A, B, J |
| Grinding | 2 | Form A |
| Total | 100 | Form A, B, C, D, E, F, |
| | | G, I, J, K, L, M |

A total of 22 anti-solvent addition experiments were carried out. About 20 mg of Compound 2 Form A was dissolved in 0.1-0.3 mL solvent to obtain a clear solution, and the solution was magnetically stirred, followed by addition of anti-solvent until precipitate appeared or the total amount of anti-solvent reached 15.0 mL. The precipitate was isolated for XRPD analysis. Clear solutions were allowed to stir at 5° C. for 4 days, and solids were then tested by XRPD. The final clear solutions were allowed to evaporate under ambient conditions. The results are summarized in Table 14:

TABLE 14

| Solvent | Anti-solvent | Solid Form |
|---|---|---|
| MeOH | IPAc | Form B |
| MeOH | MTBE | N/A |
| MeOH | toluene | Form B** |
| EtOH | EtOAc | Form D |
| EtOH | n-heptane | Form A |
| EtOH | MTBE | Form A |
| EtOH | MIBK | Form A |
| Acetic acid | EtOAc | N/A |
| Acetic acid | MTBE | N/A |
| Acetic acid | n-heptane | N/A |
| CHCl₃ | IPAc | amorphous |
| CHCl₃ | MTBE | amorphous |
| CHCl₃ | n-heptane | amorphous |
| DCM | MIBK | Form B |
| DCM | toluene | Form B** |
| DCM | EtOAc | Form A |
| H₂O | ACN | Form C** |
| H₂O | Acetone | N/A |
| DMSO | IPAc | Form B |
| DMSO | n-heptane | N/A |
| DMAc | toluene | Form B |
| DMAc | MTBE | amorphous* |

N/A: No solid was obtained after cooling at 5° C. followed by evaporation under ambient conditions.
*Amorphous sample converted into Form G after storage under ambient conditions overnight.
**Solid was obtained by evaporation of the clear solution under ambient conditions.

Slow evaporation experiments were performed under 13 conditions. In general, about 20 mg of Compound 2 Form A was dissolved in 0.5 or 1.0 mL of corresponding solvent in a 2-mL glass vial. The resulting visually clear solutions were subjected to slow evaporation under ambient conditions to induce precipitation. The solids were isolated for XRPD analysis, and the results summarized in Table 15:

TABLE 15

| Solvent (v:v) | Solid Form |
|---|---|
| MeOH | gel |
| EtOH | gel |
| IPA | Form J |
| CHCl₃ | gel |
| DCM | N/A |
| H₂O | N/A |
| THF | Form F |
| MeOH/MTBE (4:1) | gel |
| EtOH/n-heptane (4:1) | gel |
| EtOH/MTBE (4:1) | N/A |
| DCM/EtOAc (4:1) | gel |
| CHCl₃/IPAc (4:1) | gel |
| CHCl₃/n-heptane (4:1) | gel |

N/A: No solid was obtained after evaporation under ambient conditions.

Crash cooling experiments were conducted in 12 solvent systems. About 20 mg of Compound 2 Form A was suspended in 1.0 mL of solvent in a 2-mL glass vial at RT. The suspension was then heated to 50° C., equilibrated for 2 hours and filtered to a new vial using a Nylon membrane (pore size of 0.45 μm). Filtrates were cooled from 50° C. to 5° C. and stored at 5° C. without stirring. The obtained solids were kept isothermal at 5° C. before isolated for XRPD analysis. Clear solutions were evaporated to dryness at ambient conditions and then solids were tested by XRPD. The results are summarized in Table 16:

TABLE 16

| Solvent (v:v) | Solid Form |
|---|---|
| IPA | N/A |
| ACN | Form C* |
| THF | Form D |
| 2-MeTHF | Form K |
| Acetone | Form A* |
| EtOAc | N/A |
| EtOH/EtOAc (1:4) | Form M* |
| MeOH/MTBE (1:4) | N/A |
| MeOH/IPAc (1:4) | N/A |
| DMAc/MTBE (1:3) | N/A |
| DCM/MIBK (1:3) | Form A |
| CHCl₃/n-heptane (1:3) | N/A |

N/A: No solid was obtained after evaporation under ambient conditions.
*Solid was obtained by evaporation of the clear solution.

Slurry conversion experiments were conducted at room temperature in 12 different solvent systems. About 20 mg of Compound 2 Form A was suspended in 0.3 mL of solvent in each 2-mL glass vial. After the suspension was stirred for 6 days at RT, the remaining solids were isolated for XRPD analysis. Results are summarized in Table 17:

TABLE 17

| Solvent (v:v) | Solid Form |
|---|---|
| IPAc | Form G |
| MTBE | Form G |
| Toluene | Form B |
| Heptane | Form A |
| MIBK | Form B |
| EtOAc | Form L |
| ACN | Form J |
| Acetone | Form A |
| H₂O/ACN (9:991, a_{w} = 0.195) | Form J |
| Acetic acid/n-heptane (1:9) | amorphous |
| MeOH/toluene (1:9) | amorphous |
| CHCl₃/MTBE (1:9) | Form G |

Slurry conversion experiments were also conducted at 50° C. in 12 different solvent systems. About 20 mg of Compound 2 Form A was suspended in 0.3 mL of solvent in each 2-mL glass vial. After the suspension was stirred for 6 days at 50° C., the remaining solids were isolated for XRPD analysis. Results summarized in Table 18:

TABLE 18

| Solvent (v:v) | Solid Form |
|---|---|
| IPAc | Form G |
| MTBE | Form G |
| Toluene | Form B |
| Heptane | Form B |
| MIBK | Form G |
| EtOAc | Form B |
| ACN | Form J |
| Acetone | Form A |
| H₂O/ACN (9:991, a_{w} = 0.195) | Form I |
| Acetic acid/n-heptane (1:9) | amorphous |
| MeOH/toluene (1:9) | Form B |
| CHCl₃/MTBE (1:9) | Form G |

Solid vapor diffusion experiments were conducted using 13 different solvents. About 15 mg of Compound 2 Form A was weighed into a 4-mL vial, which was placed into a 20-mL vial with 3 mL of volatile solvent. The 20-mL vial was sealed with a cap and kept at RT for 6 days allowing solvent vapor to interact with sample. The solids were tested by XRPD. The results are summarized in Table 19:

TABLE 19

| Solvent | Solid Form |
|---|---|
| H₂O | N/A |
| DCM | N/A |
| EtOH | Form D |
| MeOH | N/A |
| ACN | Form C |
| THF | Form A |
| CHCl₃ | N/A |
| Acetone | Form A |
| DMF | Form E |
| EtOAc | Form A |
| 1,4-Dioxane | Form A |
| IPA | Form D |
| DMSO | N/A |

N/A: No solid was obtained after evaporation under ambient conditions.

Fourteen solution vapor diffusion experiments were conducted. Approximate 20 mg of Compound 2 Form A was dissolved in 0.5 mL of appropriate solvent to obtain a clear solution in a 4-mL vial. This solution was then placed into a 20-mL vial with 3 mL of volatile solvents. The 20-mL vial was sealed with a cap and kept at RT allowing sufficient time for organic vapor to interact with the solution. The precipitates were isolated for XRPD analysis. The results are summarized in Table 20:

TABLE 20

| Solvent | Anti-solvent | Solid Form |
|---|---|---|
| MeOH | IPAc | N/A |
| MeOH | MTBE | N/A |
| MeOH | toluene | N/A |
| EtOH | n-heptane | N/A |
| EtOH | MTBE | Form J |
| Acetic acid | n-heptane | N/A |
| CHCl₃ | IPAc | Form B |
| CHCl₃ | MTBE | N/A |
| CHCl₃ | n-heptane | N/A |
| DCM | MIBK | Form B |
| DCM | toluene | Form B |
| DCM | EtOAc | Form L + extra peak* |
| DMSO | IPAc | N/A |
| DMAc | MTBE | N/A |

N/A: No solid was obtained.
*DSC curve displayed two endotherms at 83.3° C. and 114.1° C. (peak temperature) before decomposition.

Grinding experiments was performed in two conditions with or without additive. About 15 mg of Compound 2 Form A was weighed into a mortar and then ground manually using a pestle for about 5 minutes. The solid was analyzed by XRPD and the results are summarized in Table 21:

TABLE 21

| Additive | Solid Form |
|---|---|
| N/A | Form A |
| H₂O | Form A |

N/A: No additive was added.

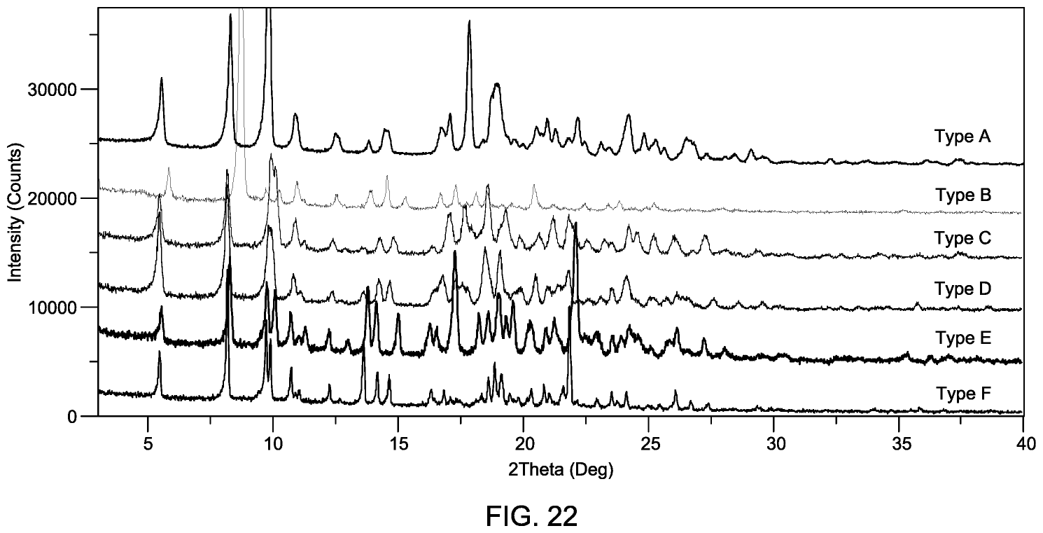

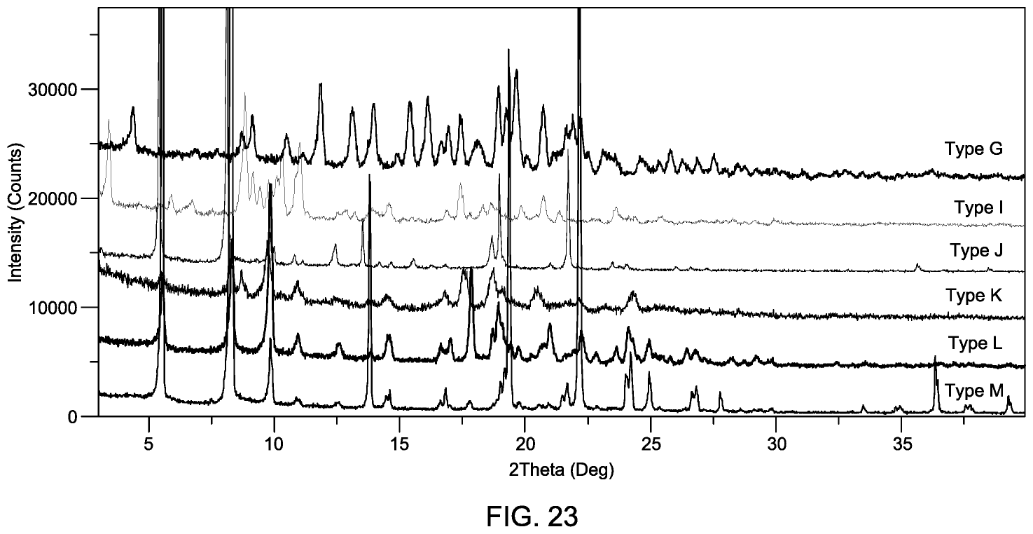

After comparison of XRPD patterns, eleven new forms were observed and characterized by TGA and DSC. Detailed characterization results are summarized in FIG. 22, FIG. 23, and Table 22.

TABLE 22

| | Weight loss | Thermal events |
|---|---|---|
| | in TGA | in DSC |
| Crystal Form | (%) | (° C., peak) |
| Form B | 2.8 | 95.7, 121.9 |
| Form G | 0.2 | 120.1 |
| Form I | 1.4 | 126.8 |
| Form K | 4.7 | 121.1 |
| Form M | 0.2 | 90.4 |
| Form A | 5.1 | 84.0 |
| Form C | 6.2 | 59.2, 76.7 |
| Form D | 6.1 | 89.5, 107.1 |
| Form E | 2.5 | 76.5 |
| Form F | 3.9 | 74.6 |
| Form J | 2.3 | 57.9, 84.1 |
| Form L | 5.9 | 73.1, 80.8, 87.5 |

### Example 9—Characterization of Solid Forms of Compound 2 Compound 2 Form A

Compound 2 Form A (i.e., Compound 1 Succinate Form A) was prepared and characterized as described above in Example 3.

### Compound 2 Form B

Compound 2 Form B was prepared by anti-solvent addition, slurrying at room temperature or at 50° C., or by solution vapor diffusion, as reported above.

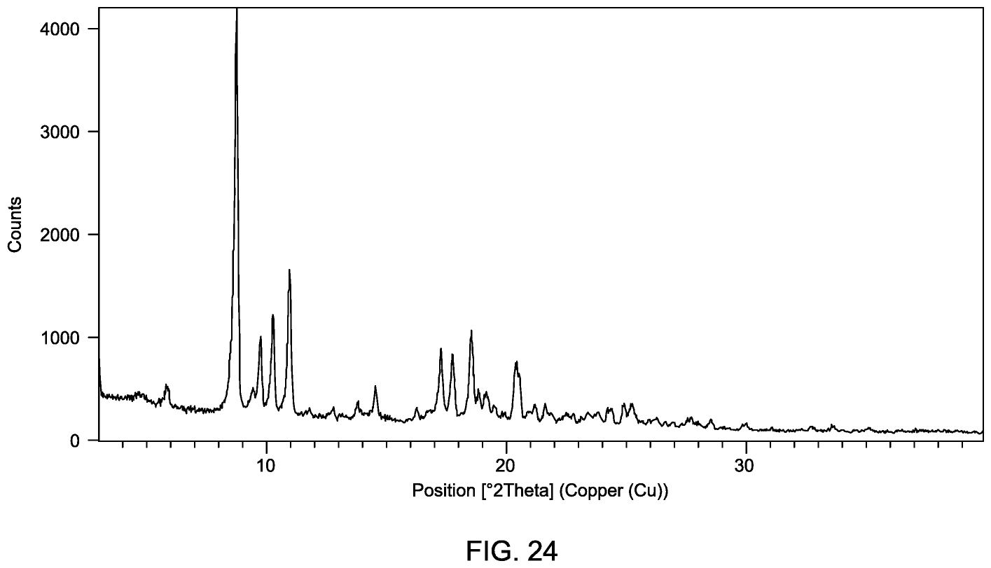

The X-ray powder diffraction pattern of the crystalline Compound 2 Form B is depicted in FIG. 24, and the corresponding data is summarized below:

| 2 theta | d-spacing |
|---|---|
| 4.7 | 18.7 |
| 5.9 | 15.1 |
| 8.7 | 10.1 |
| 9.7 | 9.1 |
| 10.3 | 8.6 |
| 11.0 | 8.1 |
| 11.7 | 7.5 |
| 13.8 | 6.4 |
| 14.5 | 6.1 |
| 16.2 | 5.5 |
| 17.2 | 5.1 |
| 17.7 | 5.0 |
| 18.5 | 4.8 |
| 18.8 | 4.7 |
| 19.1 | 4.6 |
| 20.4 | 4.4 |
| 20.6 | 4.3 |
| 21.2 | 4.2 |
| 21.6 | 4.1 |
| 22.6 | 3.9 |
| 23.8 | 3.7 |
| 24.3 | 3.7 |
| 24.9 | 3.6 |
| 25.2 | 3.5 |
| 26.1 | 3.4 |
| 26.9 | 3.3 |
| 27.7 | 3.2 |
| 28.5 | 3.1 |
| 30.0 | 3.0 |
| 32.7 | 2.7 |
| 33.6 | 2.7 |
| 35.1 | 2.6 |

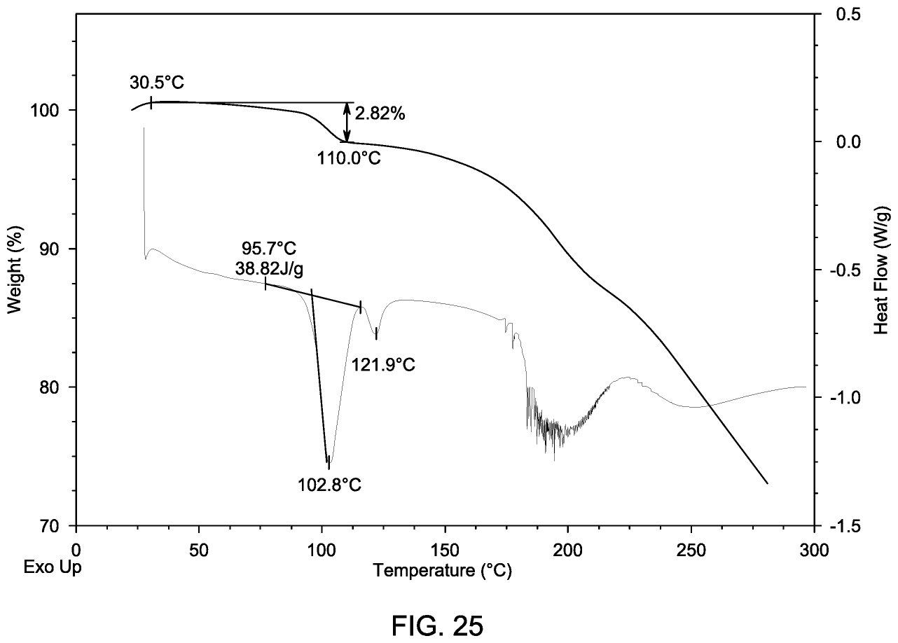

As shown by TGA and DSC curves in FIG. 25, Form B showed a weight loss of 2.8% up to 110° C. and two endothermic peaks at 102.8° C. and 121.9° C. (peak temperature) before decomposition.

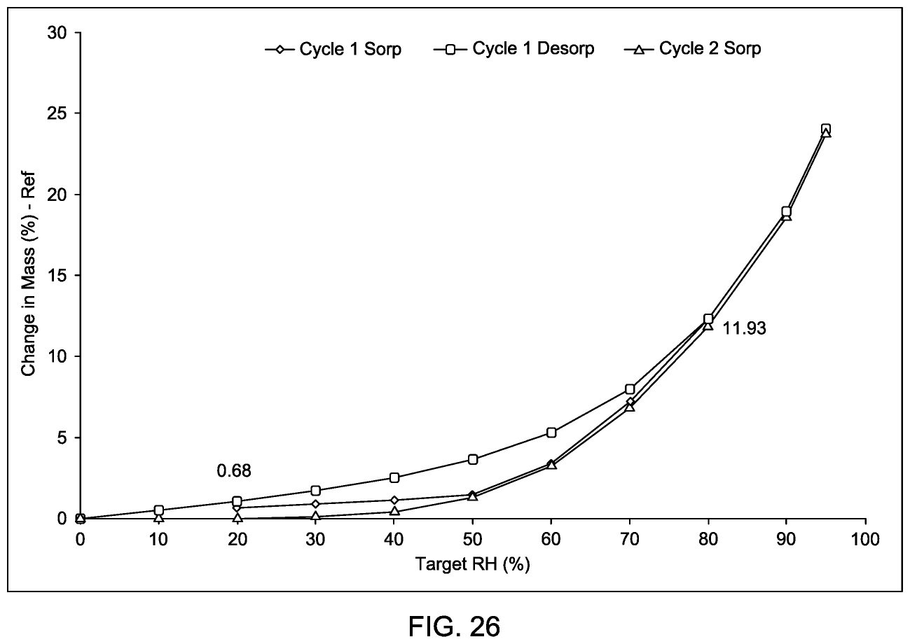

A DVS analysis of Form B is reported in FIG. 26. Form B is highly hygroscopic with a large absorption up to 80% RH. After the DVS test, Form B becomes amorphous.

### Compound 2 Form C

Compound 2 Form C was prepared by anti-solvent addition, crash cooling, or solid vapor diffusion, as reported above

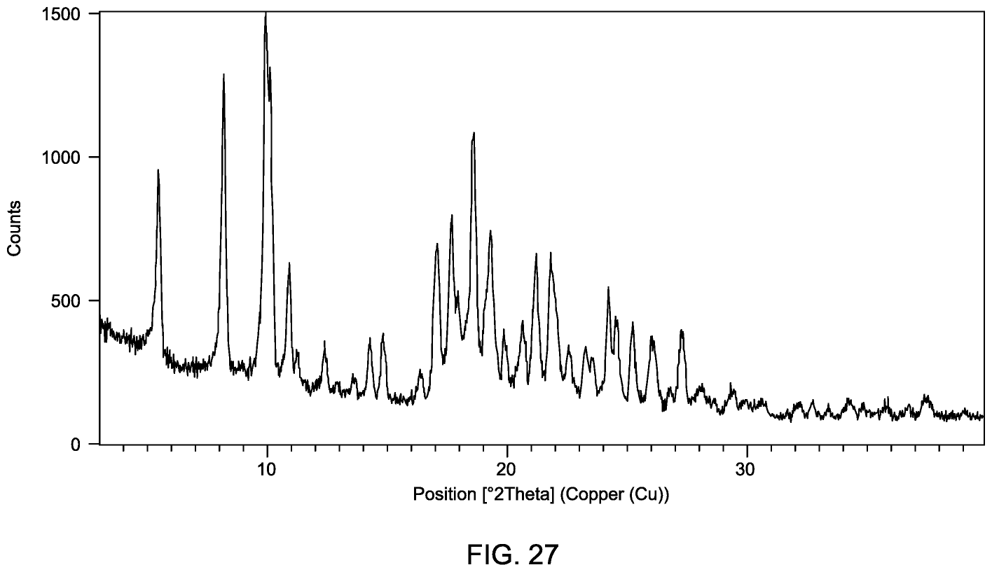

The X-ray powder diffraction pattern of the crystalline Compound 2 Form C is depicted in FIG. 27, and the corresponding data is summarized below:

| 2 theta | d-spacing |
|---|---|
| 5.4 | 16.3 |
| 8.1 | 10.9 |
| 9.9 | 8.9 |
| 10.1 | 8.7 |
| 10.9 | 8.1 |
| 12.4 | 7.1 |
| 14.2 | 6.2 |
| 14.8 | 6.0 |
| 16.4 | 5.4 |
| 17.1 | 5.2 |
| 17.7 | 5.0 |
| 18.6 | 4.8 |
| 19.3 | 4.6 |
| 19.9 | 4.5 |
| 20.7 | 4.3 |
| 21.2 | 4.2 |
| 21.8 | 4.1 |
| 22.6 | 3.9 |
| 23.2 | 3.8 |
| 24.2 | 3.7 |
| 24.6 | 3.6 |
| 25.2 | 3.5 |
| 26.0 | 3.4 |
| 27.3 | 3.3 |
| 29.3 | 3.0 |
| 30.0 | 3.0 |
| 34.3 | 2.6 |
| 37.5 | 2.4 |

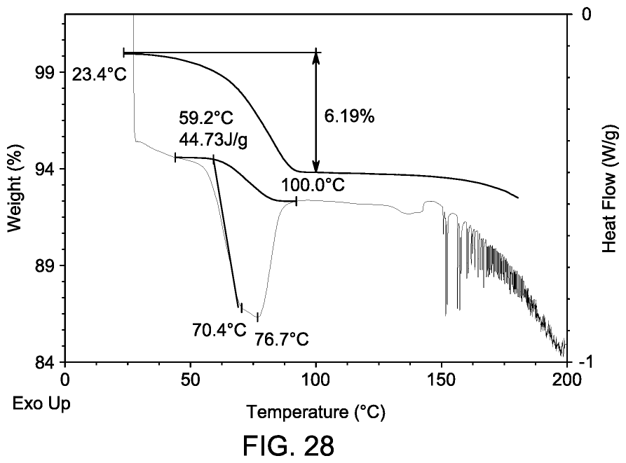

As shown by TGA and DSC curves in FIG. 28, Form C showed a weight loss of 6.2% up to 100° C. and one endotherm at 59.2° C. (onset temperature) before decomposition.

### Compound 2 Form D

Compound 2 Form D was prepared by anti-solvent addition, crash cooling, or solid vapor diffusion, as reported above

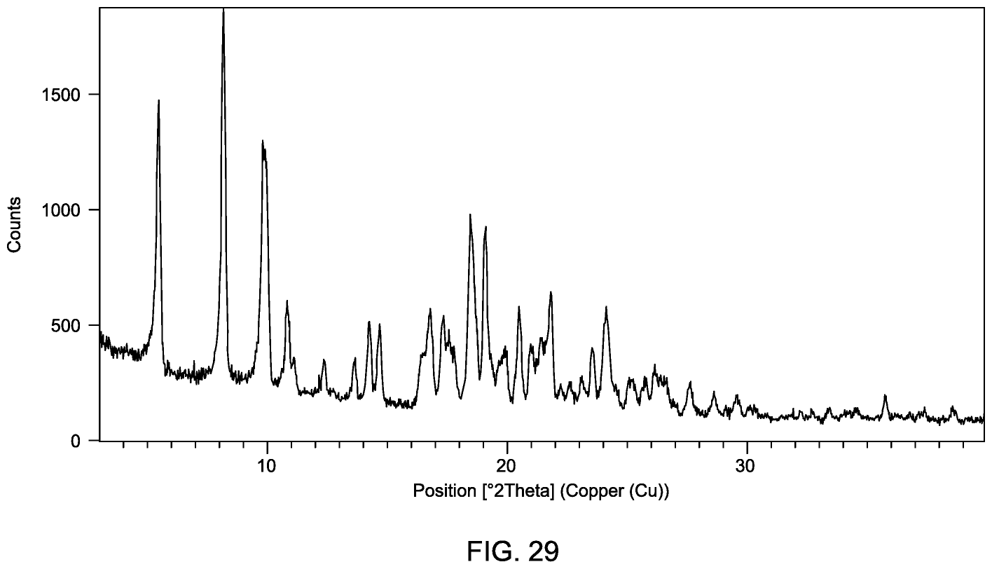

The X-ray powder diffraction pattern of the crystalline Compound 2 Form D is depicted in FIG. 29, and the corresponding data is summarized below:

| 2 theta | d-spacing |
|---|---|
| 5.4 | 16.2 |
| 8.1 | 10.9 |
| 9.8 | 9.0 |
| 10.0 | 8.9 |
| 10.8 | 8.2 |
| 12.4 | 7.2 |
| 13.6 | 6.5 |
| 14.2 | 6.2 |
| 14.7 | 6.0 |
| 16.8 | 5.3 |
| 17.3 | 5.1 |
| 18.5 | 4.8 |
| 19.1 | 4.7 |
| 19.9 | 4.5 |
| 20.5 | 4.3 |
| 20.9 | 4.2 |
| 21.8 | 4.1 |
| 23.5 | 3.8 |
| 24.1 | 3.7 |
| 25.1 | 3.5 |
| 25.7 | 3.5 |
| 26.1 | 3.4 |
| 27.6 | 3.2 |
| 29.6 | 3.0 |
| 33.4 | 2.7 |
| 35.8 | 2.5 |
| 38.6 | 2.3 |

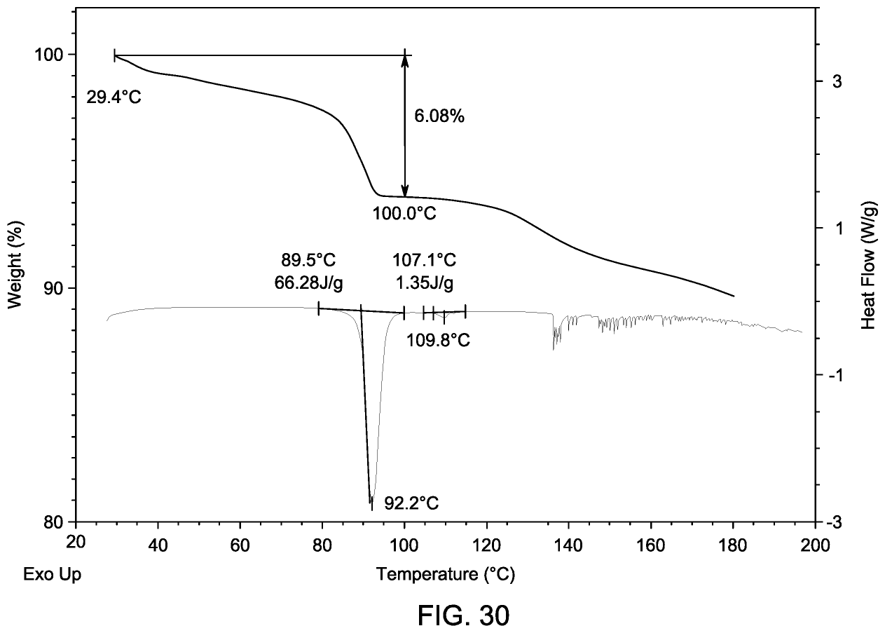

As shown by TGA and DSC curves in FIG. 30, Form D showed a weight loss of 6.18% up to 100° C. and one endotherm at 89.5° C. (onset temperature) before decomposition.

### Compound 2 Form E

Compound 2 Form E was prepared by solid vapor diffusion, as reported above.

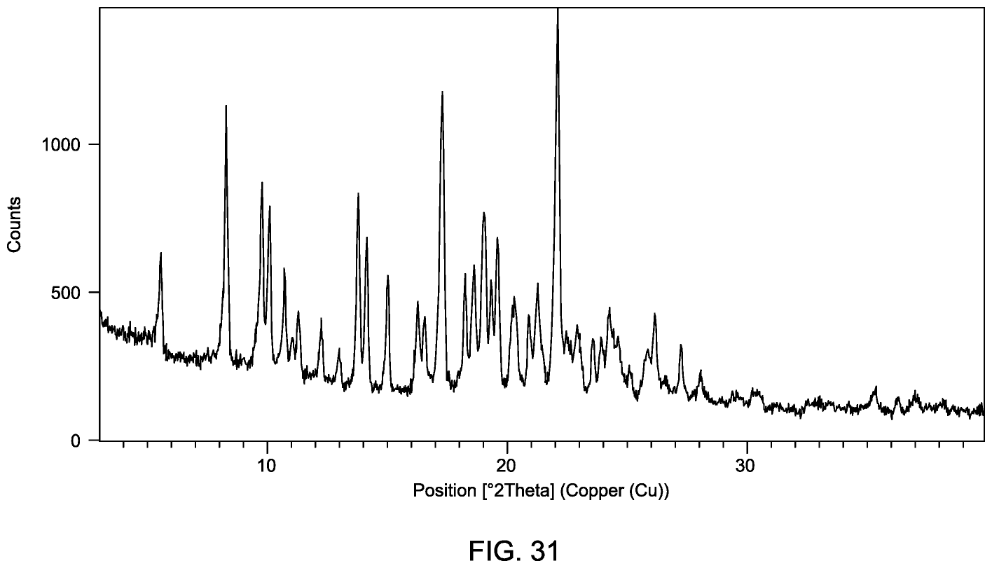

The X-ray powder diffraction pattern of the crystalline Compound 2 Form E is depicted in FIG. 31, and the corresponding data is summarized below:

| 2 theta | d-spacing |
|---|---|
| 5.5 | 16.0 |
| 8.3 | 10.7 |
| 9.8 | 9.1 |
| 10.1 | 8.8 |
| 10.7 | 8.3 |
| 11.3 | 7.8 |
| 12.2 | 7.2 |
| 13.0 | 6.8 |
| 13.8 | 6.4 |
| 14.1 | 6.3 |
| 15.0 | 5.9 |
| 16.3 | 5.4 |
| 16.5 | 5.4 |
| 17.3 | 5.1 |
| 18.2 | 4.9 |
| 18.6 | 4.8 |
| 19.0 | 4.7 |
| 19.6 | 4.5 |
| 20.3 | 4.4 |
| 20.9 | 4.3 |
| 21.2 | 4.2 |
| 22.1 | 4.0 |
| 22.9 | 3.9 |
| 23.6 | 3.8 |
| 24.2 | 3.7 |
| 26.2 | 3.4 |
| 27.2 | 3.3 |
| 28.1 | 3.2 |
| 30.4 | 2.9 |
| 32.8 | 2.7 |
| 35.3 | 2.5 |
| 36.3 | 2.5 |
| 37.0 | 2.4 |

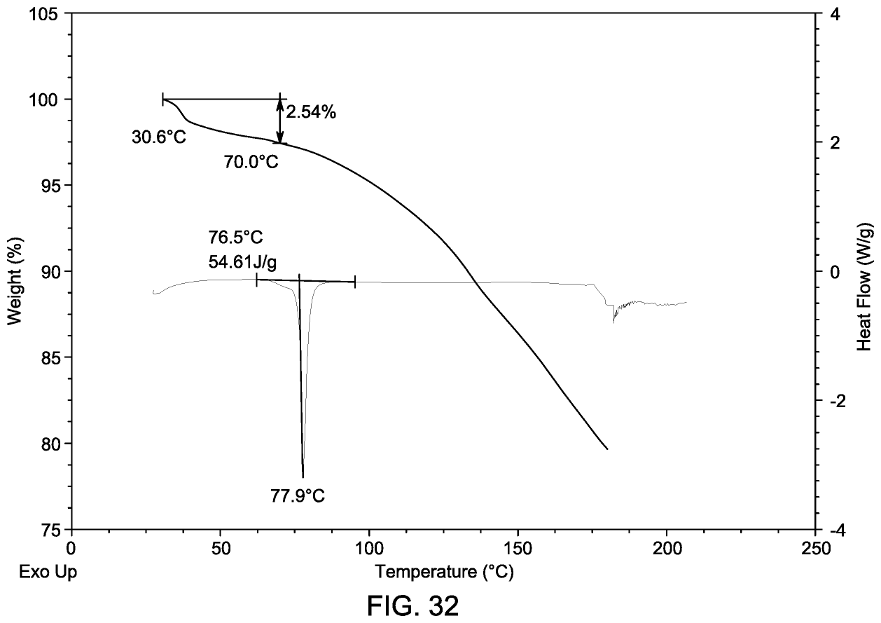

As shown by TGA and DSC curves in FIG. 32, Form E showed a weight loss of 2.5% up to 70° C. and one endotherm at 76.5° C. (onset temperature) before decomposition.

### Compound 2 Form F

Compound 2 Form F was prepared by slow evaporation, as reported above.

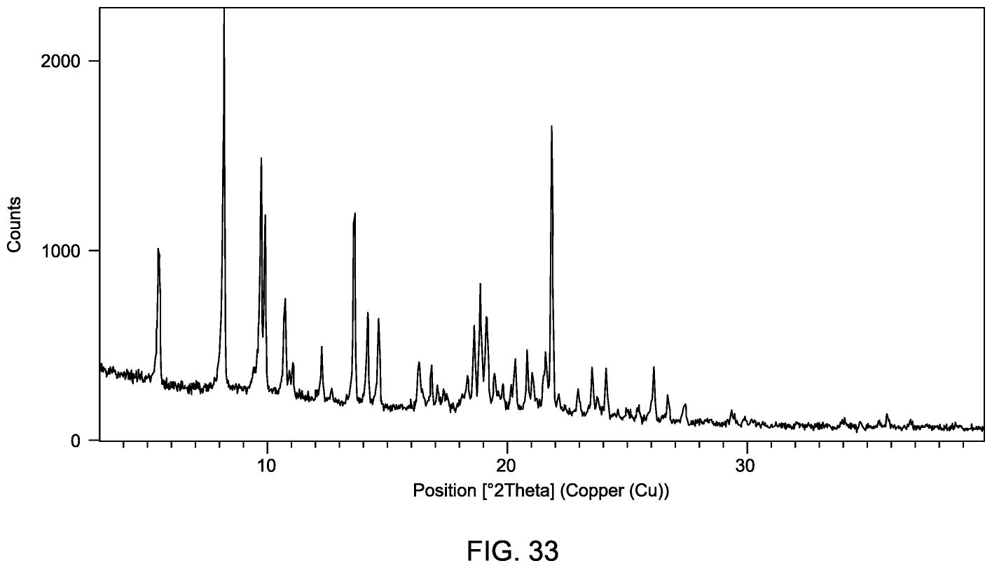

The X-ray powder diffraction pattern of the crystalline Compound 2 Form F is depicted in FIG. 33, and the corresponding data is summarized below:

| 2 theta | d-spacing |
|---|---|
| 5.5 | 16.1 |
| 8.2 | 10.8 |
| 9.7 | 9.1 |
| 9.9 | 8.9 |
| 10.7 | 8.3 |
| 12.3 | 7.2 |
| 13.6 | 6.5 |
| 14.2 | 6.2 |
| 14.6 | 6.0 |
| 16.3 | 5.4 |
| 16.8 | 5.3 |
| 18.6 | 4.8 |
| 18.9 | 4.7 |
| 19.1 | 4.6 |
| 19.5 | 4.6 |
| 20.3 | 4.4 |
| 20.8 | 4.3 |
| 21.0 | 4.2 |
| 21.8 | 4.1 |
| 22.9 | 3.9 |
| 23.5 | 3.8 |
| 23.8 | 3.7 |
| 24.1 | 3.7 |
| 26.1 | 3.4 |
| 26.7 | 3.3 |
| 27.4 | 3.3 |
| 29.4 | 3.0 |

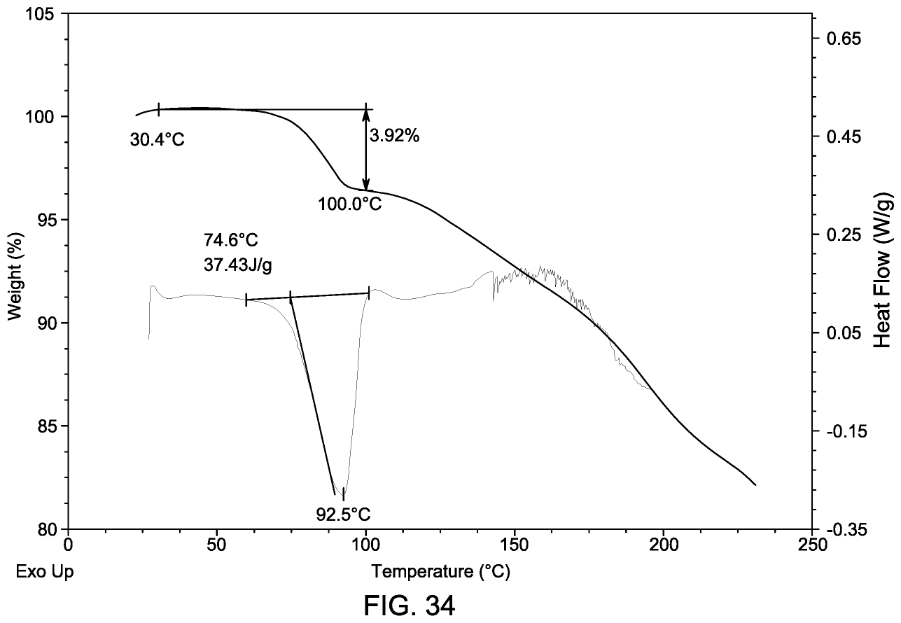

As shown by TGA and DSC curves in FIG. 34, Form F showed a weight loss of 3.9% up to 100° C. and one endotherm at 74.6° C. (onset temperature) before decomposition.

### Compound 2 Form G

Compound 2 Form G was prepared by at least the following methods:

Method A. About 20 mg of Compound 2 Form A was suspended in 0.3 mL of a solvent. The suspension was stirred for 6 days at room temperature. The solids were then isolated and analyzed by XRPD. Example solvents that generate Form G include IPAc, MTBE, and CHCl₃/MTBE (1:9).

Method B. About 20 mg of Compound 2 Form A was suspended in 0.3 mL of a solvent. The suspension was stirred for 6 days at 50° C. The solids were then isolated and analyzed by XRPD. Example solvents that generate Form G include IPAc, MTBE, MIBK, and CHCl₃/MTBE (1:9).

Method C: About 500 g of Compound 2 Form A was added to a reaction vessel with about 9980 mL (20:1 molar ratio) of isopropyl acetate. The suspension was heated to 50° C. and stirred for 18 hours. The temperature was raised to 70° C. and the solvent was distilled until the final volume of the suspension was about 5 L (about 10:1 molar ratio). The mixture was cooled to room temperature over the course of 1 hour or more. The mixture was then stirred for about 14 hours. The suspension was then filtered under vacuum and washed with isopropyl acetate and dried under vacuum to yield Compound 2 Form G.

Method D: About 20 mg of Compound 2 Form A was dissolved in 0.1-0.3 mL of a solvent to obtain a clear solution. The solution was magnetically stirred followed by addition of an anti-solvent until a precipitate appeared or the total solvent volume reached 15.0 mL. The solids were isolated and stored overnight under ambient conditions.

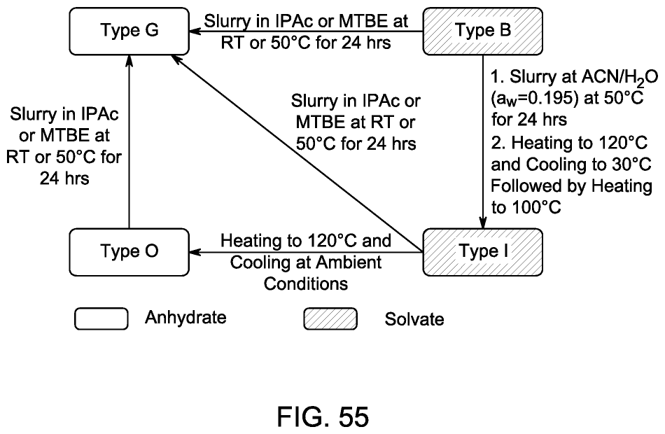

Additionally, Forms B, I, and O can be converted to Form G according as illustrated in FIG. 55. In particular, any of Forms B, I, or O can be used in place of Form A under Method A to obtain Form G.

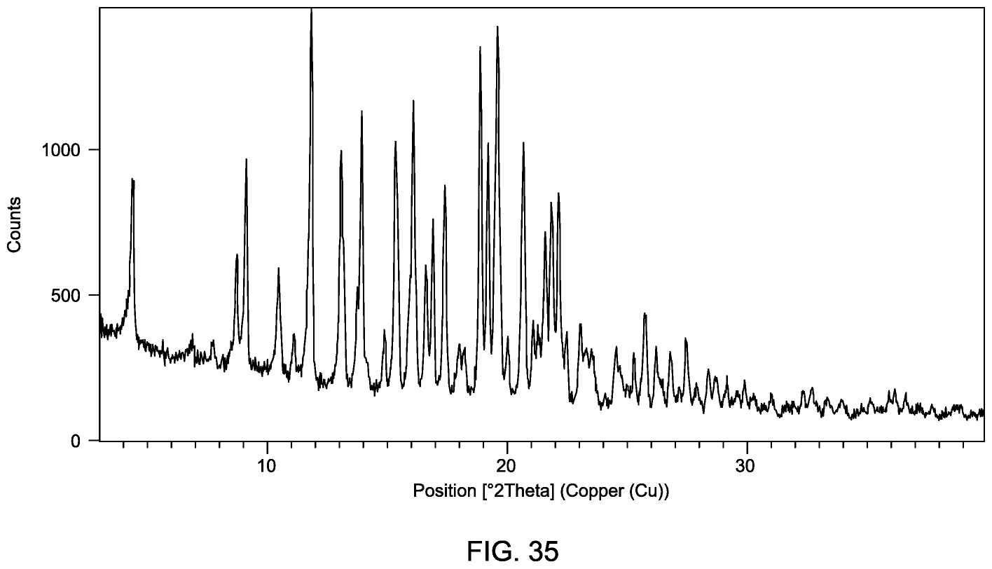

The X-ray powder diffraction pattern of the crystalline Compound 2 Form G is depicted in FIG. 35, and the corresponding data is summarized below:

| 2 theta | d-spacing |
|---|---|
| 4.4 | 20.1 |
| 6.8 | 13.0 |
| 7.7 | 11.4 |
| 8.7 | 10.1 |
| 9.1 | 9.7 |
| 10.5 | 8.4 |
| 11.1 | 7.9 |
| 11.8 | 7.5 |
| 13.1 | 6.8 |
| 13.9 | 6.4 |
| 14.9 | 5.9 |
| 15.3 | 5.8 |
| 16.1 | 5.5 |
| 16.6 | 5.3 |
| 16.9 | 5.2 |
| 17.4 | 5.1 |
| 18.0 | 4.9 |
| 18.2 | 4.9 |
| 18.9 | 4.7 |
| 19.2 | 4.6 |
| 19.6 | 4.5 |
| 20.0 | 4.4 |
| 20.7 | 4.3 |
| 21.1 | 4.2 |
| 21.6 | 4.1 |
| 21.8 | 4.1 |
| 22.1 | 4.0 |
| 22.5 | 4.0 |
| 23.0 | 3.9 |
| 23.5 | 3.8 |
| 24.5 | 3.6 |
| 25.3 | 3.5 |
| 25.8 | 3.5 |
| 26.2 | 3.4 |
| 26.8 | 3.3 |
| 27.5 | 3.2 |
| 27.9 | 3.2 |
| 28.4 | 3.1 |
| 28.7 | 3.1 |
| 29.1 | 3.1 |
| 29.9 | 3.0 |
| 31.0 | 2.9 |
| 32.7 | 2.7 |
| 33.3 | 2.7 |
| 34.0 | 2.6 |
| 35.2 | 2.6 |
| 36.1 | 2.5 |
| 36.6 | 2.5 |
| 38.8 | 2.3 |

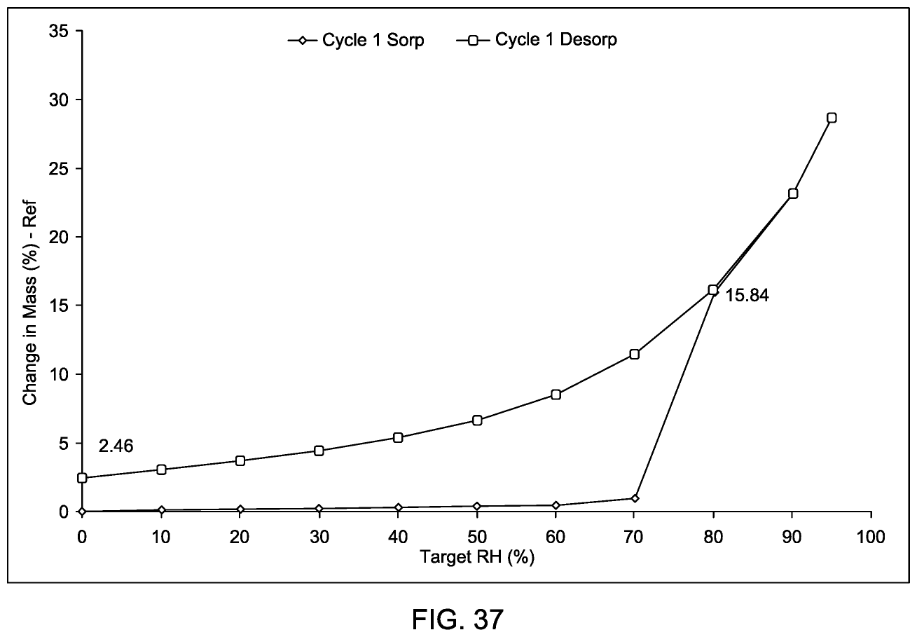

The moisture sorption/desorption property of Compound 2 From G is depicted in FIG. 37. Results of the DVS testing indicate that Compound 2 Form G absorbed only 0.76% water by weight below 70% relative humidity (RH), and absorbed about 25.19% (wt/wt) water above 70% RH. Referring to the DVS isotherm plot in FIG. 37, the lower curve corresponds to the sorption of water by increasing relative humidity for a sample of Compound 2 Form G, and the upper curve corresponds to desorption by decreasing relative humidity of a sample of Compound 2 Form G. As noted, results of the DVS testing indicate that Compound 2 Form G absorbed only 0.76% water by weight below 70% relative humidity (RH), and absorbed about 25.19% (wt/wt) water above 70% RH.

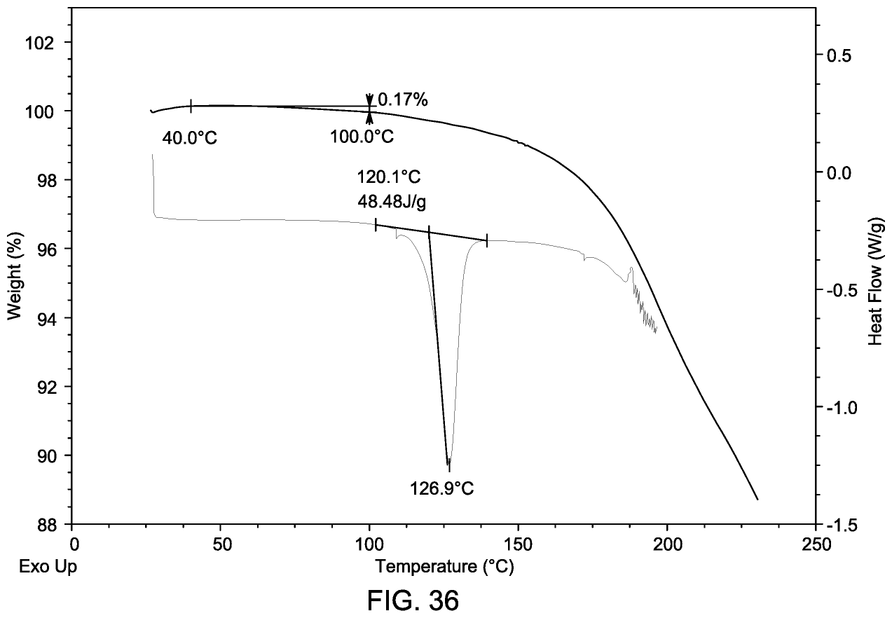

Thermogravimetric analysis (TGA) and differential scanning calorimetry (DSC) thermograms are shown in FIG. 36. An initial weight loss of 0.2% is observed in the TGA thermogram from initial temperature to 150° C., and was followed by decomposition. The DSC thermogram exhibited an endotherm at about 127° C. (onset temperature).

At storage conditions below 75% RH, Form G showed no significant change in physical form after 42 days by XRPD, PLM or DSC/TGA (Table 23). The stability samples stored at ambient condition for 42 days, 25° C./75% RH for 42 days, and 50° C. for 21 days showed good flow properties by visual assessment.

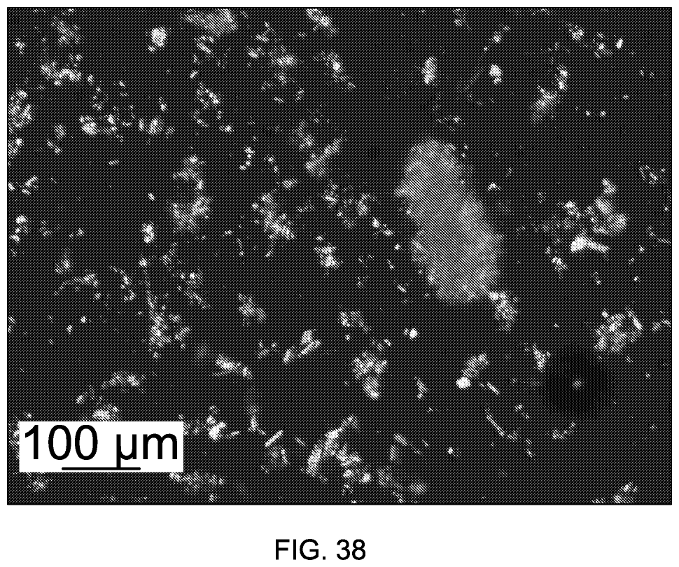

Polarized-light microscopy (PLM) is shown in FIG. 38, and shows that Compound 2 Form G is composed of 1-50 μm birefringent irregular and rectangular plates.

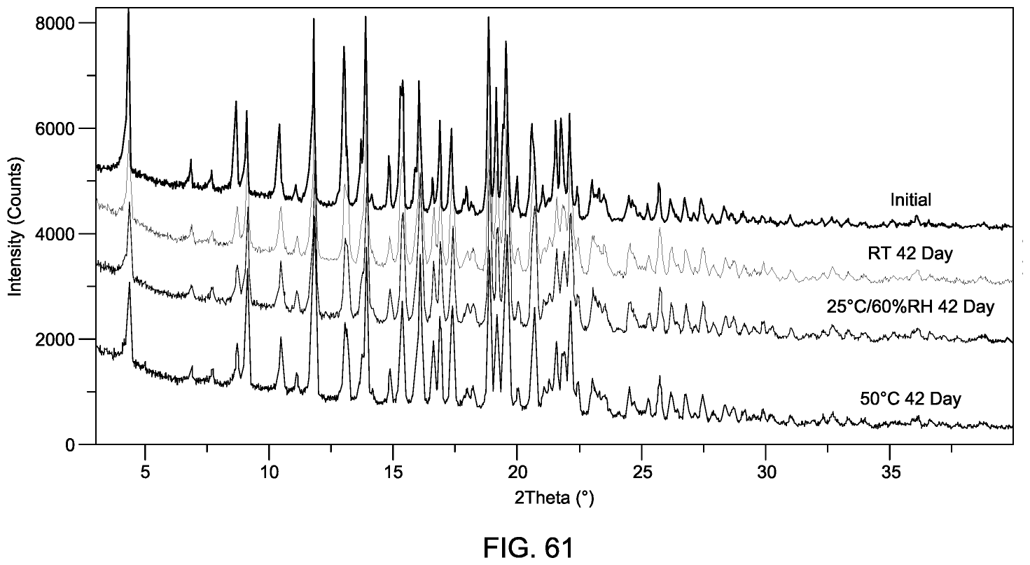

The stability of Compound 2 Form G was tested over 42 days at varying conditions and is reported below in Table 23 and in FIG. 61:

TABLE 23

| | | DSC | | |
|---|---|---|---|---|
| | | endotherm | | TGA % |
| Storage | Time | Temp | ΔH | weight loss |
| Conditions | (days) | (° C.) | (J/g) | before 150° C. |
| Initial | 0 | 125.51 | 63.56 | 0.5459 |
| ambient, closed | 1 | 125.59 | 62.59 | 0.5454 |
| | 3 | 125.61 | 63.01 | 0.5338 |
| | 7 | 125.41 | 63.00 | 0.5540 |
| | 14 | 125.52 | 63.86 | 0.4133 |
| | 21 | 125.38 | 67.51 | 0.8085 |
| | 42 | 125.35 | 66.56 | 0.4578 |
| 25° C./60% RH | 1 | 125.46 | 66.76 | 0.4580 |
| open | 3 | 125.61 | 63.86 | 0.5433 |
| | 7 | 125.68 | 64.19 | 0.5129 |
| | 14 | 125.64 | 65.95 | 0.5232 |
| | 21 | 125.94 | 64.03 | 0.6097 |
| | 42 | 126.50 | 57.76 | 0.6016 |
| 50° C., closed | 1 | 125.53 | 67.41 | 0.4716 |
| | 3 | 125.97 | 59.90 | 0.4670 |
| | 7 | 125.97 | 65.47 | 0.5022 |
| | 14 | 125.16 | 64.27 | 0.5893 |
| | 21 | 125.21 | 64.69 | 0.5702 |
| | 42* | 121.80 | 51.32 | 0.7707 |

As can be seen, stability samples at 50° C. experienced a temperature excursion. On Day 38, stability samples at 50° C. were exposed to temperatures of approximately 85° C. for 24 hours. However, even with increased stress on this sample, the samples appeared physically stable, with no significant change to XRPD, DSC/TGA or PLM. By visual assessment, the 50° C. sample did show a slight reduction in flowability after being exposed to higher temperatures.

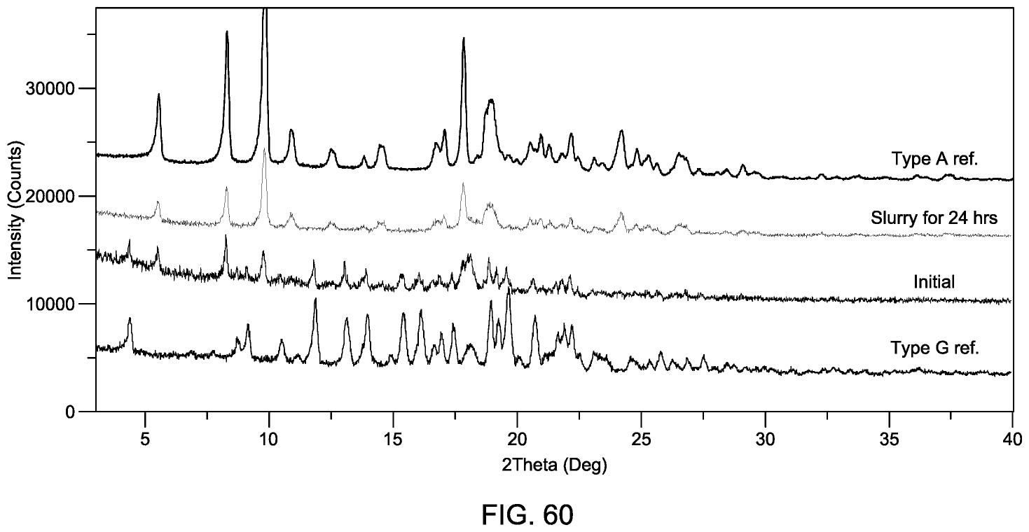

To test the solubility of Form A and G, Form A was dissolved in acetone to get a saturated solution, then 0.4 mg of Form G in 0.6 mL was added into the saturated solution, solid was still observed after vibration and ultra-sonication. The suspension was heated to 50° C. until the solid was mostly dissolved, then the solution was kept in a cold room (5° C.) for about 1 hour, and the precipitated solid was analyzed by XRPD. The result displayed in FIG. 60 showed that most peaks came from Form A and only two peaks belong to Form G.

The chemical stability of Form G was determined using HPLC-UV at 275 nm and 254 nm.

As seen in Table 9 (reported under Compound 2 Form A, above), Form G showed no significant chemical degradation after 42 days at temperatures below 40° C. All stability samples showed 95-107% claim of the expected Compound 2 content by HPLC-UV analysis at 275 nm. Stability samples at ambient conditions and 25° C./60% RH open for 42 days showed no reduction in purity and no significant impurity increase by HPLC-UV analysis at 254 nm.

For reference, the chemical stability of Compound 2 Form A was also evaluated at 25° C./60% RH for 42 days and did not show significant chemical degradation by HPLC-UV.

### Compound 2 Form I

Compound 2 Form I was prepared by slurrying at room 50° C., as reported above.

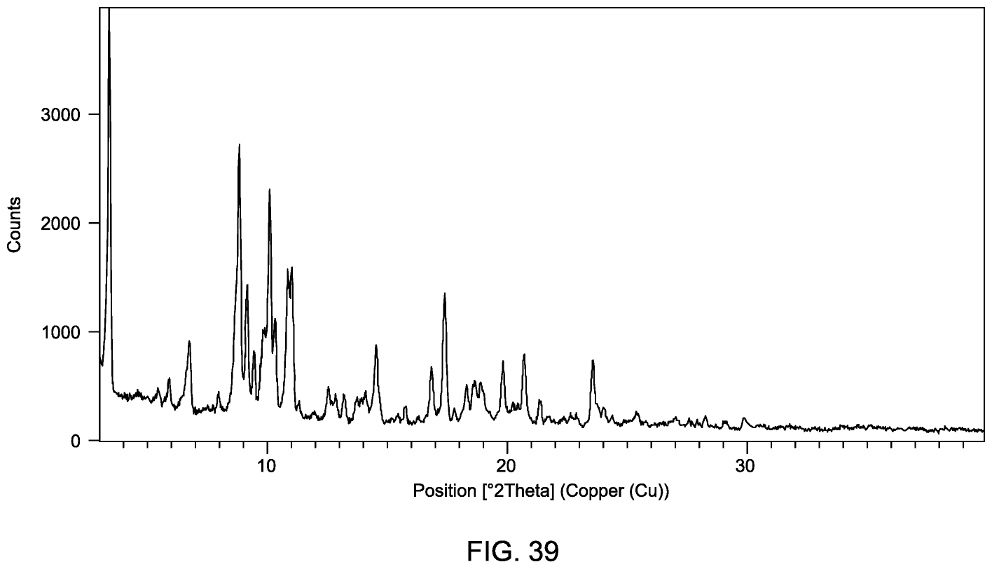

The X-ray powder diffraction pattern of the crystalline Compound 2 Form I is depicted in FIG. 39, and the corresponding data is summarized below:

| 2 theta | d-spacing |
|---|---|
| 3.4 | 25.8 |
| 5.9 | 15.0 |
| 6.7 | 13.1 |
| 8.0 | 11.1 |
| 8.8 | 10.0 |
| 9.2 | 9.7 |
| 9.5 | 9.4 |
| 9.8 | 9.0 |
| 10.1 | 8.8 |
| 10.3 | 8.6 |
| 10.8 | 8.2 |
| 11.0 | 8.0 |
| 12.5 | 7.1 |
| 12.9 | 6.9 |
| 13.2 | 6.7 |
| 13.9 | 6.4 |
| 14.5 | 6.1 |
| 15.7 | 5.6 |
| 16.8 | 5.3 |
| 17.4 | 5.1 |
| 18.3 | 4.8 |
| 18.9 | 4.7 |
| 19.8 | 4.5 |
| 20.7 | 4.3 |
| 21.4 | 4.2 |
| 23.6 | 3.8 |
| 25.4 | 3.5 |
| 27.0 | 3.3 |
| 28.3 | 3.2 |
| 29.1 | 3.1 |
| 29.9 | 3.0 |

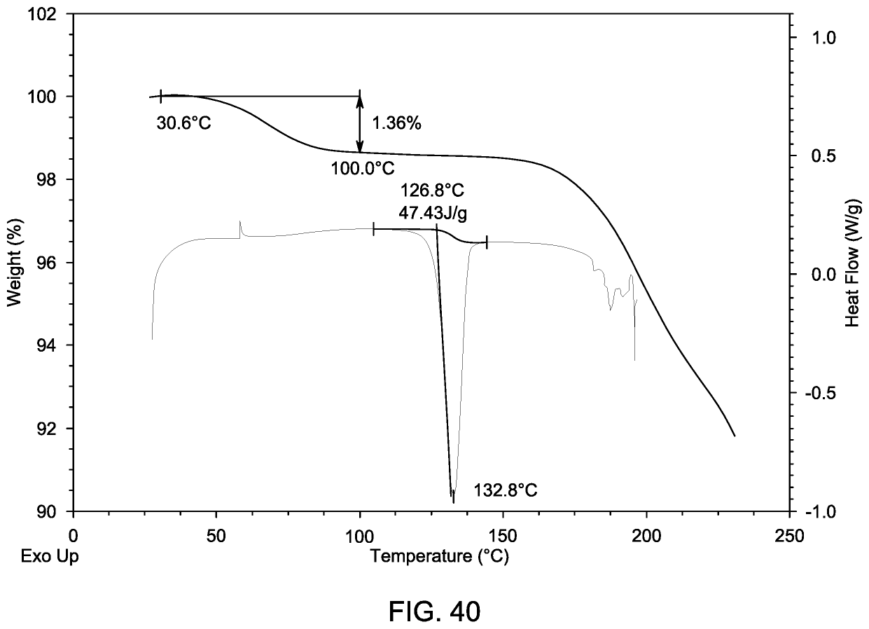

As shown by TGA and DSC curves in FIG. 40, Form I showed a weight loss of 1.4% up to 100° C. and one endotherm at 126.8° C. (onset temperature) before decomposition.

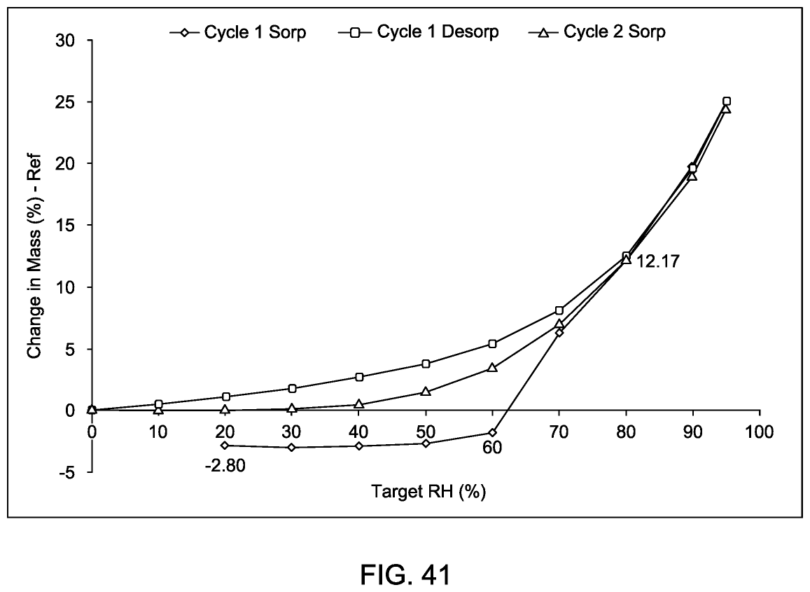

A DVS analysis of Form I is reported in FIG. 41. DVS analysis shows that Form I exhibits a minor weight change before 60% RH while more absorption was observed at up to 80% RH, indicating Form I's high hygroscopicity. After DVS, Form I becomes amorphous.

### Compound 2 Form J

Compound 2 Form J was prepared by slurrying at 50° C. or by solution vapor diffusion, as reported above.

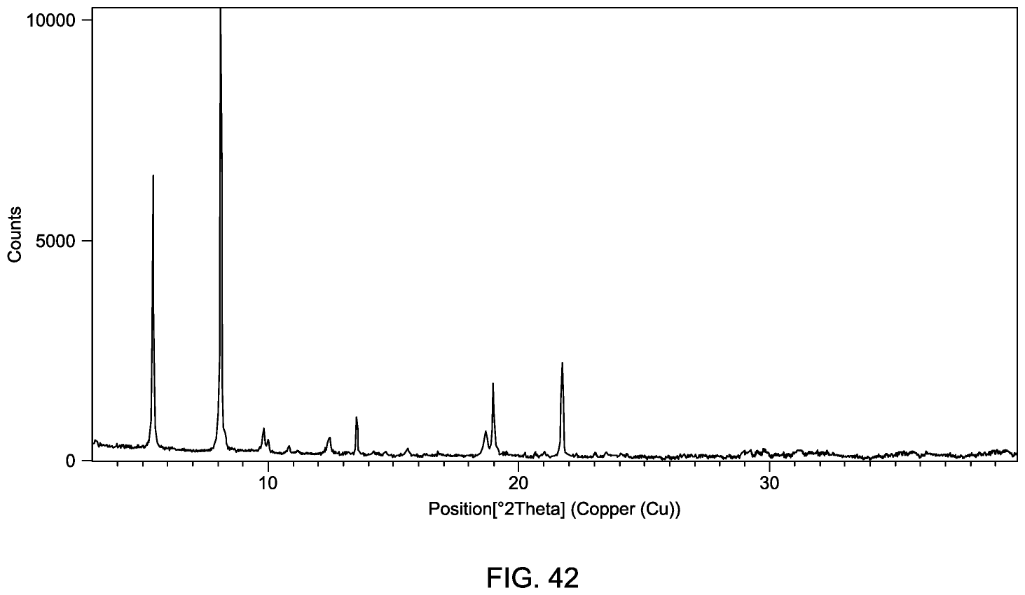

The X-ray powder diffraction pattern of the crystalline Compound 2 Form J is depicted in FIG. 42, and the corresponding data is summarized below:

| 2 theta | d-spacing |
|---|---|
| 5.4 | 16.3 |
| 8.1 | 10.9 |
| 9.8 | 9.0 |
| 10.0 | 8.8 |
| 10.8 | 8.2 |
| 12.4 | 7.1 |
| 13.5 | 6.5 |
| 15.6 | 5.7 |
| 18.7 | 4.7 |
| 19.0 | 4.7 |
| 21.0 | 4.2 |
| 21.7 | 4.1 |
| 23.5 | 3.8 |
| 35.6 | 2.5 |
| 38.5 | 2.3 |

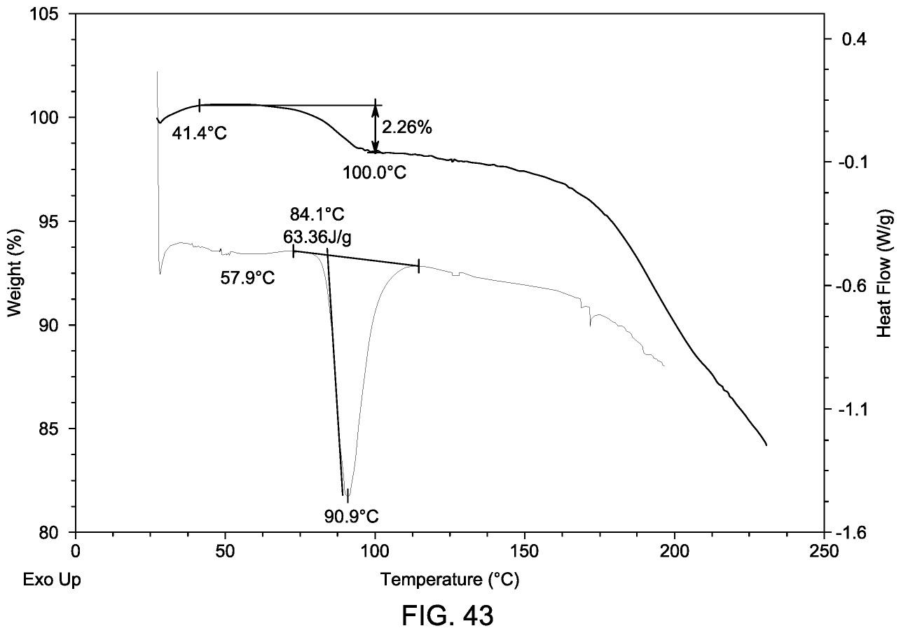

As shown by TGA and DSC curves in FIG. 43, Form J showed a weight loss of 2.3% up to 100° C. and one endothermic peak at 57.9° C. (peak temperature) and another at 84.1° (peak temperature) before decomposition.

### Compound 2 Form K

Compound 2 Form K was prepared by crash cooling, as reported above.

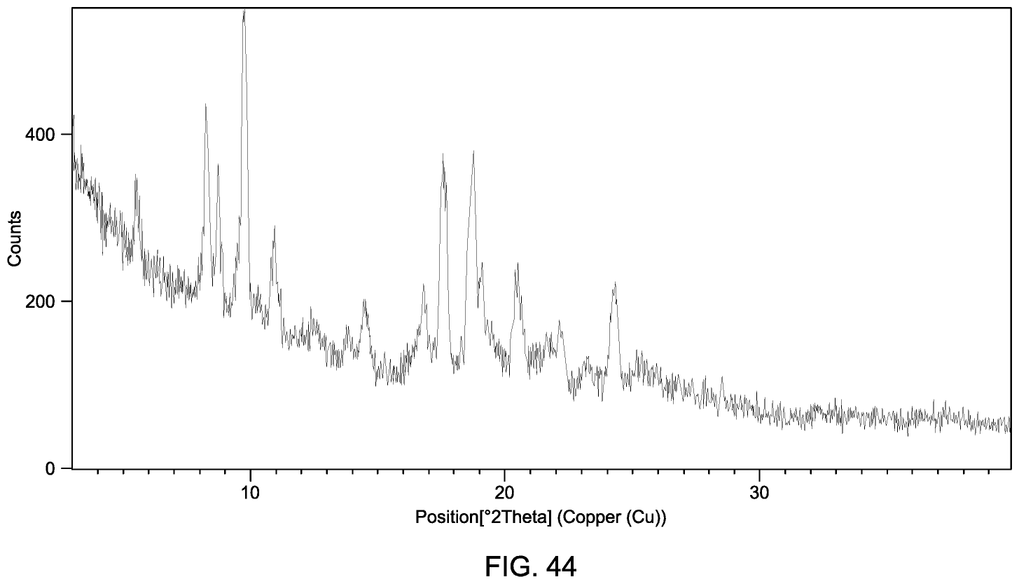

The X-ray powder diffraction pattern of the crystalline Compound 2 Form K is depicted in FIG. 44, and the corresponding data is summarized below

| 2 theta | d-spacing |
|---|---|
| 5.5 | 15.9 |
| 8.3 | 10.7 |
| 8.7 | 10.2 |
| 9.7 | 9.1 |
| 10.9 | 8.1 |
| 14.5 | 6.1 |
| 16.8 | 5.3 |
| 17.6 | 5.0 |
| 18.7 | 4.7 |
| 20.5 | 4.3 |
| 22.2 | 4.0 |
| 24.4 | 3.7 |

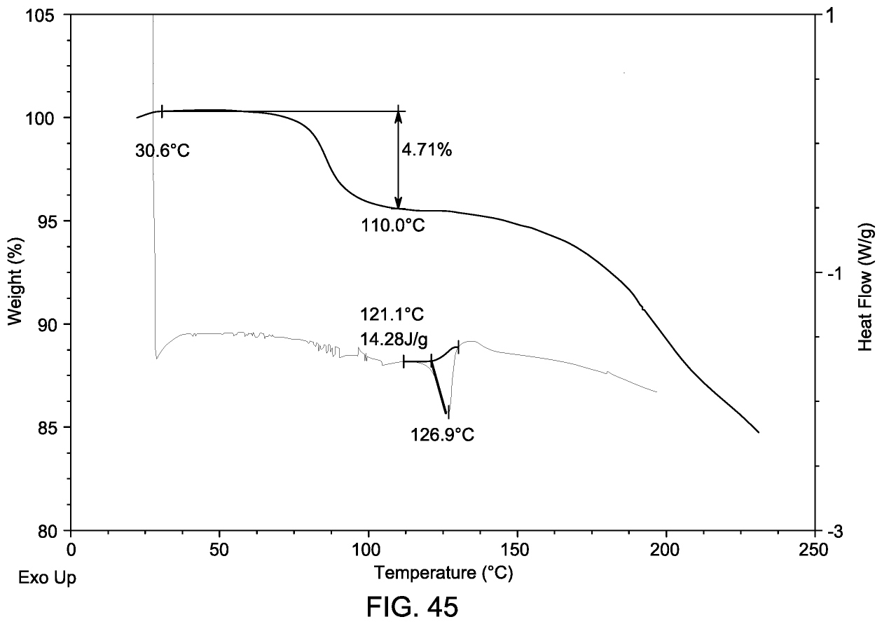

As shown by TGA and DSC curves in FIG. 45, Form K showed a weight loss of 4.7% up to 110° C. and one endotherm at 121.1° C. (onset temperature) before decomposition.

### Compound 2 Form L

Compound 2 Form L was prepared by at least by a slurrying at room temperature, as reported above.

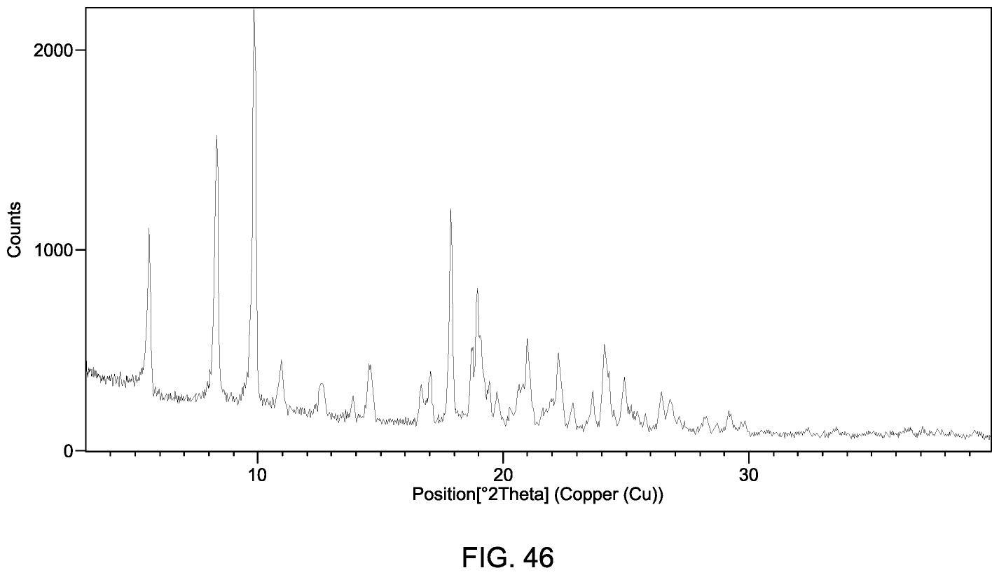

The X-ray powder diffraction pattern of the crystalline Compound 2 Form L is depicted in FIG. 46, and the corresponding data is summarized below:

| 2 theta | d-spacing |
|---|---|
| 5.5 | 16.0 |
| 8.3 | 10.6 |
| 9.8 | 9.0 |
| 11.0 | 8.1 |
| 12.6 | 7.0 |
| 13.9 | 6.4 |
| 14.6 | 6.1 |
| 16.6 | 5.3 |
| 17.0 | 5.2 |
| 17.8 | 5.0 |
| 18.7 | 4.7 |
| 18.9 | 4.7 |
| 19.5 | 4.6 |
| 19.8 | 4.5 |
| 21.0 | 4.2 |
| 22.2 | 4.0 |
| 22.8 | 3.9 |
| 23.7 | 3.8 |
| 24.1 | 3.7 |
| 25.0 | 3.6 |
| 25.8 | 3.5 |
| 26.5 | 3.4 |
| 26.8 | 3.3 |
| 28.2 | 3.2 |
| 29.2 | 3.1 |

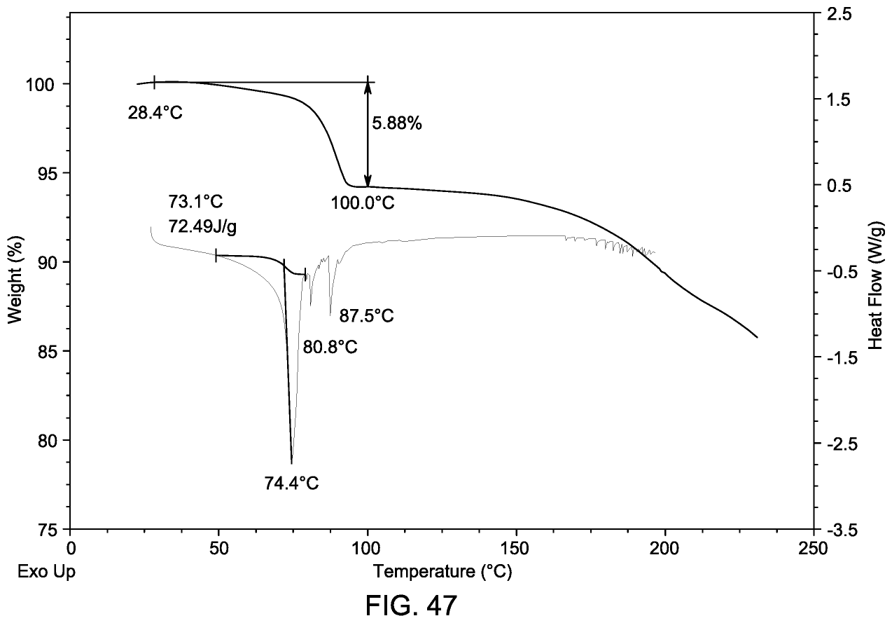

As shown by TGA and DSC curves in FIG. 47, Form L showed a weight loss of 5.9% up to 100° C. and three endothermic peaks at 74.4° C., 80.8° C. and 87.5° C. (peak temperatures) before decomposition.

### Compound 2 Form M

Compound 2 Form M was prepared by evaporating Compound 2 Form A from a solution in EtOH/EtOAc (1:4 v/v) that had been crash cooled.

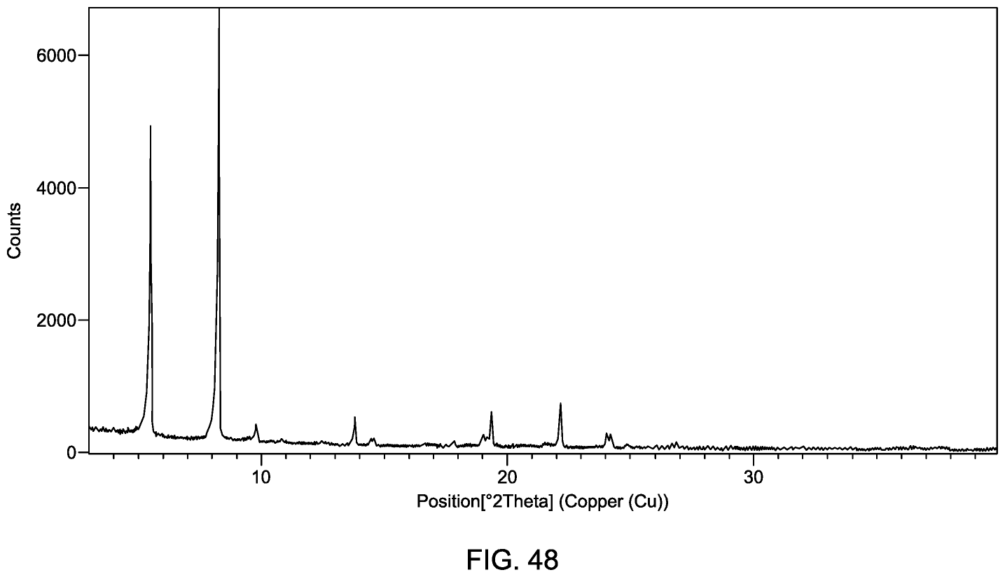

The X-ray powder diffraction pattern of the crystalline Compound 2 Form M is depicted in FIG. 48, and the corresponding data is summarized below:

| 2 theta | d-spacing |
|---|---|
| 5.5 | 16.0 |
| 8.3 | 10.7 |
| 9.8 | 9.0 |
| 10.8 | 8.2 |
| 12.4 | 7.1 |
| 13.8 | 6.4 |
| 14.5 | 6.1 |
| 16.7 | 5.3 |
| 17.8 | 5.0 |
| 19.0 | 4.7 |
| 19.4 | 4.6 |
| 21.5 | 4.1 |
| 22.1 | 4.0 |
| 24.0 | 3.7 |
| 24.2 | 3.7 |
| 24.9 | 3.6 |
| 26.7 | 3.3 |
| 36.4 | 2.5 |
| 37.7 | 2.4 |

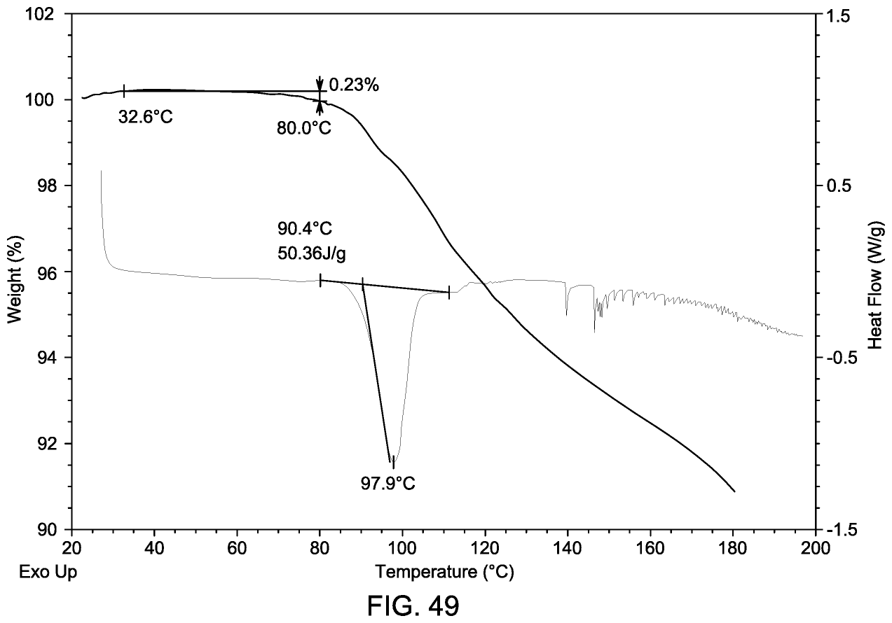

As shown by TGA and DSC curves in FIG. 49, Form M showed a weight loss of 0.2% before 80° C. and one endotherm at 90.4° C. (onset temperature) before decomposition.

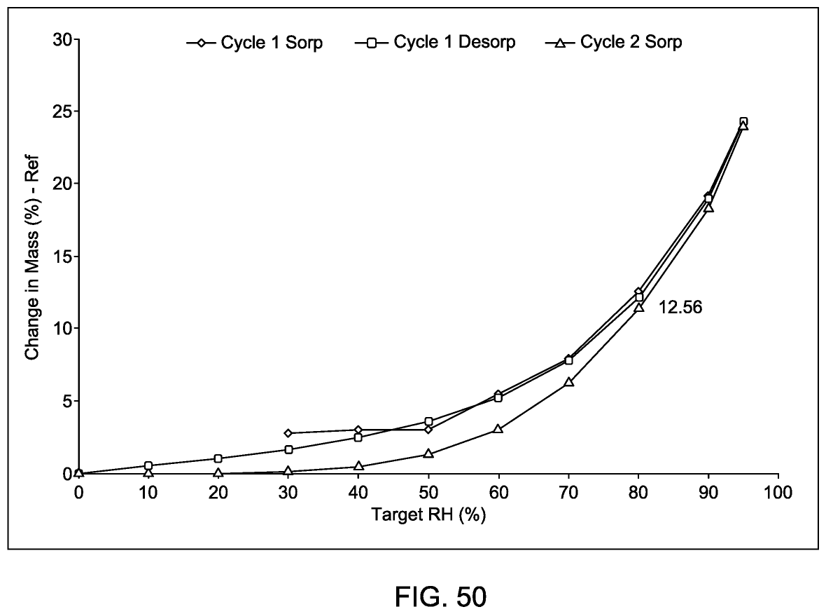

A DVS analysis of Form M is reported in FIG. 50. Form M is highly hygroscopic with a large absorption up to 80% RH. After the DVS test, Form M becomes amorphous.

### Compound 2 Form O

Compound 2 Form O was prepared by slurrying Compound 2 Form A at 50° C. in IPAc.

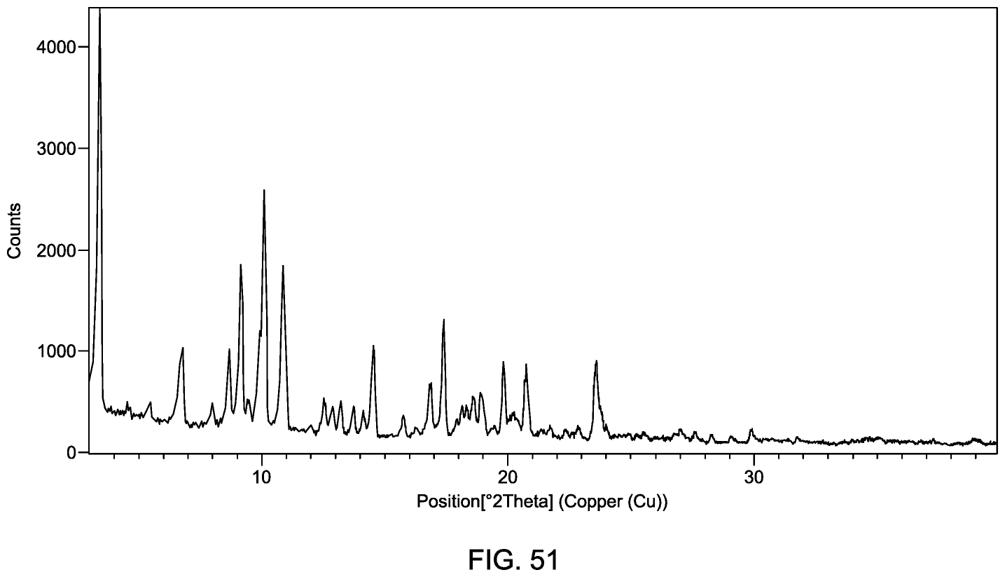

The X-ray powder diffraction pattern of the crystalline Compound 2 Form O is depicted in FIG. 51, and the corresponding data is summarized below:

| 2 theta | d-spacing |
|---|---|
| 3.4 | 25.7 |
| 4.6 | 19.2 |
| 5.5 | 16.2 |
| 6.8 | 13.0 |
| 8.0 | 11.1 |
| 8.7 | 10.2 |
| 9.2 | 9.7 |
| 9.4 | 9.4 |
| 9.9 | 8.9 |
| 10.1 | 8.7 |
| 10.9 | 8.1 |
| 12.0 | 7.4 |
| 12.5 | 7.1 |
| 12.9 | 6.9 |
| 13.2 | 6.7 |
| 13.7 | 6.4 |
| 14.1 | 6.3 |
| 14.6 | 6.1 |
| 15.8 | 5.6 |
| 16.8 | 5.3 |
| 17.4 | 5.1 |
| 18.1 | 4.9 |
| 18.3 | 4.8 |
| 18.6 | 4.8 |
| 18.9 | 4.7 |
| 19.8 | 4.5 |
| 20.2 | 4.4 |
| 20.7 | 4.3 |
| 21.7 | 4.1 |
| 22.3 | 4.0 |
| 22.8 | 3.9 |
| 23.6 | 3.8 |
| 27.0 | 3.3 |
| 27.6 | 3.2 |
| 28.3 | 3.2 |
| 29.1 | 3.1 |
| 29.9 | 3.0 |
| 31.8 | 2.8 |
| 34.6 | 2.6 |
| 35.1 | 2.6 |

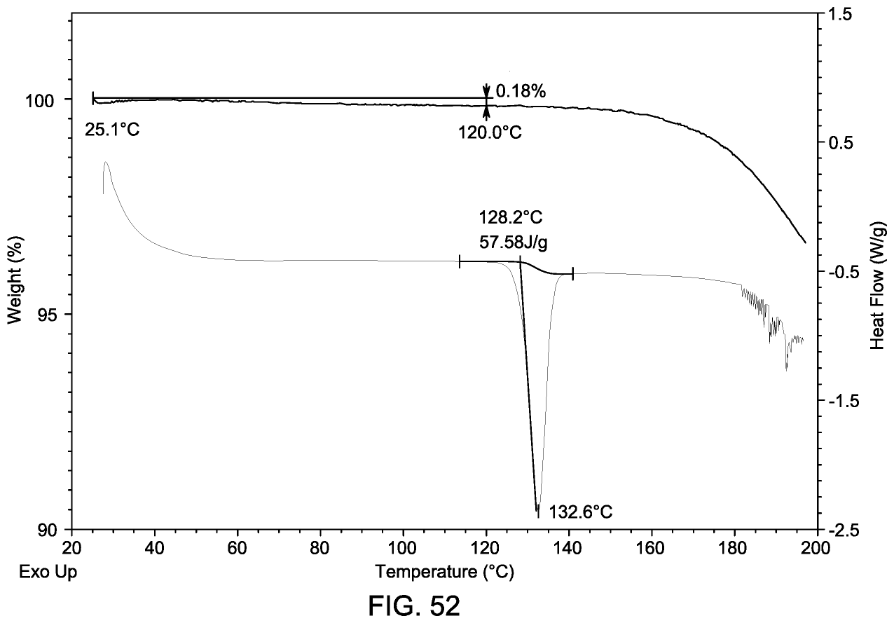

As shown by TGA and DSC curves in FIG. 52, Form O showed a weight loss of 0.2% before 120° C. and one endotherm at 128.2° C. (onset temperature) before decomposition.

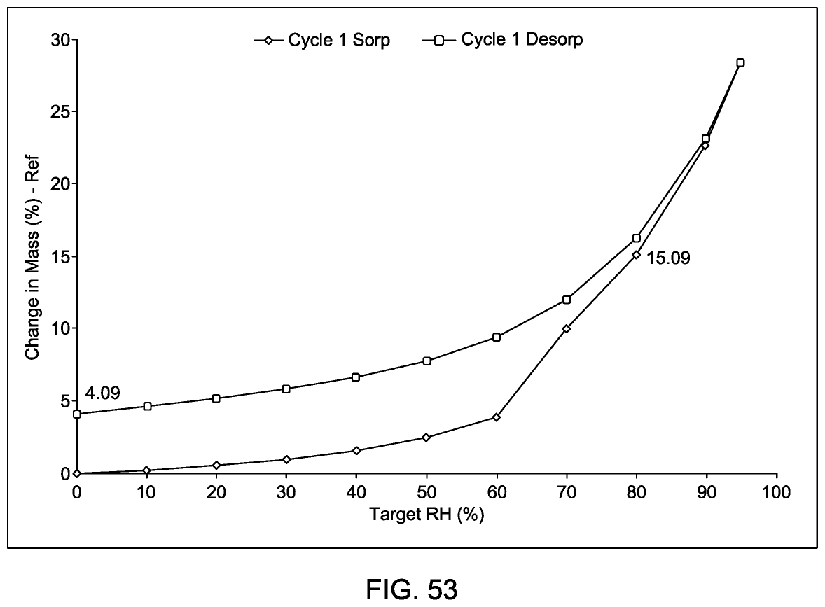

A DVS analysis of Form O is reported in FIG. 53. Form O is highly hygroscopic with a large absorption up to 80% RH. After the DVS test, Form O becomes amorphous. Compound 2 Form P

Compound 2 Form P was prepared by slurrying Compound 2 Form A at 50° C. in IPAc.

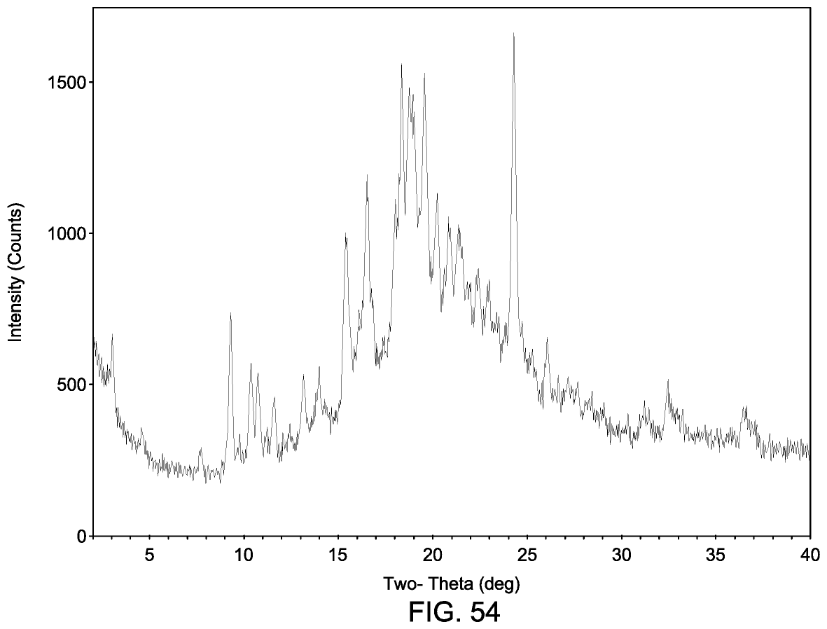

The X-ray powder diffraction pattern of the crystalline Compound 2 Form P is depicted in FIG. 54, and the corresponding data is summarized below:

| 2 theta | d-spacing |
|---|---|
| 4.3 | 20.6 |
| 6.8 | 13.0 |
| 7.6 | 11.7 |
| 8.6 | 10.3 |
| 9.0 | 9.8 |
| 10.3 | 8.5 |
| 11.0 | 8.0 |
| 11.7 | 7.5 |
| 12.3 | 7.2 |
| 12.9 | 6.8 |
| 13.8 | 6.4 |
| 14.0 | 6.3 |
| 14.8 | 6.0 |
| 15.2 | 5.8 |
| 16.0 | 5.5 |
| 16.5 | 5.4 |
| 16.8 | 5.3 |
| 17.3 | 5.1 |
| 17.9 | 5.0 |
| 18.1 | 4.9 |
| 18.8 | 4.7 |
| 19.1 | 4.6 |
| 19.5 | 4.6 |
| 19.9 | 4.5 |
| 20.5 | 4.3 |
| 20.9 | 4.2 |
| 21.5 | 4.1 |
| 21.7 | 4.1 |
| 22.0 | 4.0 |
| 22.3 | 4.0 |
| 23.0 | 3.9 |
| 23.1 | 3.8 |
| 23.4 | 3.8 |
| 24.4 | 3.7 |
| 24.6 | 3.6 |
| 25.2 | 3.5 |
| 25.6 | 3.5 |
| 26.1 | 3.4 |
| 26.7 | 3.3 |
| 27.1 | 3.3 |
| 27.3 | 3.3 |
| 27.8 | 3.2 |
| 28.2 | 3.2 |
| 28.9 | 3.1 |
| 29.4 | 3.0 |
| 29.8 | 3.0 |
| 30.1 | 3.0 |
| 30.5 | 2.9 |
| 30.8 | 2.9 |
| 31.5 | 2.8 |
| 32.1 | 2.8 |
| 32.5 | 2.7 |
| 33.1 | 2.7 |
| 33.8 | 2.6 |
| 35.0 | 2.6 |
| 36.0 | 2.5 |
| 36.5 | 2.5 |
| 36.9 | 2.4 |
| 37.5 | 2.4 |
| 38.7 | 2.3 |
| 39.6 | 2.3 |

### Example 10—Stability Evaluation Between Compound 2 Form G and Compound 2 Form O

The slurry competition experiments between the two anhydrates, Form G and Form O, were performed in two solvents, IPAc and MTBE, at RT and 50° C. for stability evaluation. Form O was dissolved in IPAc and MTBE respectively to get saturated solutions. Then, similar masses of Form G and Form O were added into a 2-mL vial, upon which the corresponding saturated solution was added to give the slurry, which was stirred at RT or 50° C. for 24 hours. The solids collected were analyzed by XRPD to confirm the form change.

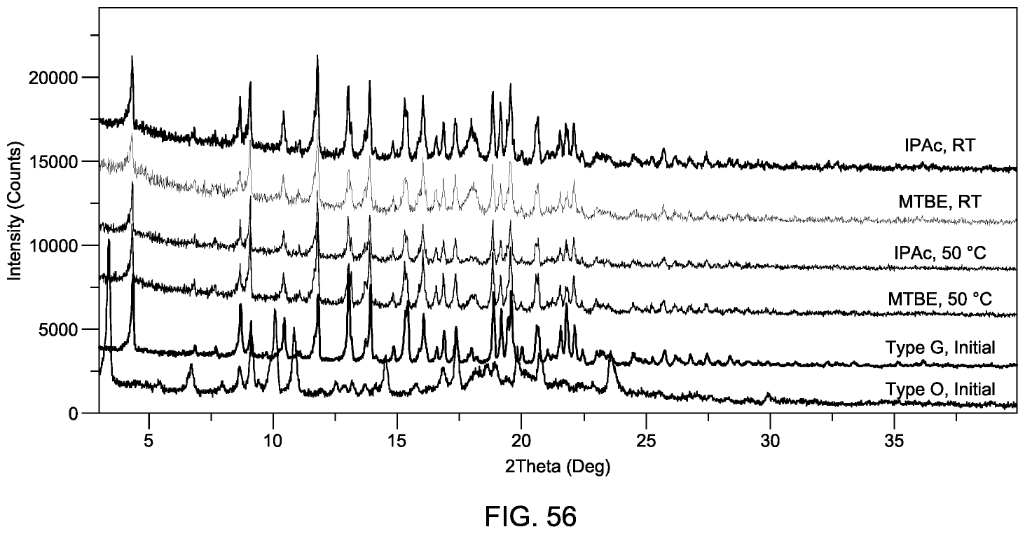

As summarized in Table 24 and FIG. 56, the solids from the slurries converted to Form G completely.

TABLE 24

Mass (mg)

| Form G | Form O | Solvent | Temperature | Results |
|---|---|---|---|---|
| 6.0 | 6.0 | IPAc | RT | Form G |
| 6.5 | 6.1 | MTBE | RT | Form G |
| 6.2 | 6.1 | IPAc | 50° C. | Form G |
| 6.6 | 6.5 | MTBE | 50° C. | Form G |

### Example 11—Stability Evaluation Between Compound 2 Form B, Compound 2 Form I, and Compound 2 Form G

To further study the form identity of Form B and Form I, slurry turnover experiments were performed in two solvents with the addition of Form G. Form B was dissolved in IPAc and MTBE respectively to get saturated solutions. Similar mass of Form B, G and I were weighed into a 2-mL vial, upon which the corresponding saturated solution was added to give the slurry, which was stirred at RT or 50° C. After slurry for 24 hours, the solids obtained were analyzed by XRPD.

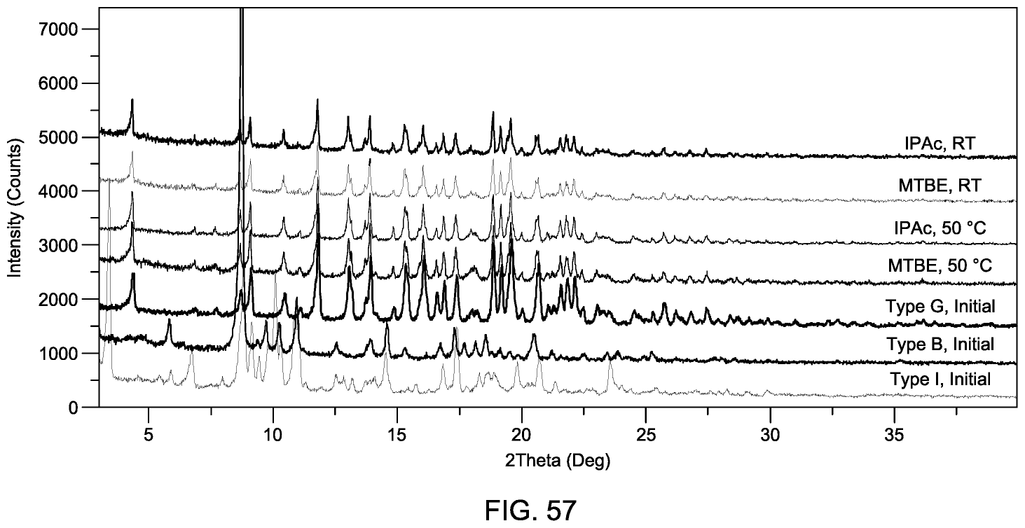

As summarized in Table 25 and FIG. 57, the slurries started with a mixture of Form B, G and I converted to Form G completely.

TABLE 25

| Mass (mg) | | | | | |
|---|---|---|---|---|---|
| Form B | Form G | Form I | Solvent | Temp. | Results |
| 5.9 | 6.3 | 5.5 | IPAc | RT | Form G |
| 6.2 | 6.3 | 5.6 | MTBE | RT | Form G |
| 5.8 | 6.4 | 5.0 | IPAc | 50° C. | Form G |
| 5.8 | 6.3 | 5.3 | MTBE | 50° C. | Form G |

### Example 12—Identification of the Critical a_{w} Between Compound 2 Form G and Compound 2 Form I

To investigate the critical a_{w} between hydrated Form I and the stable anhydrate, Form G, slurry turnover of Form I and Form G was conducted in a co-solvent system (H₂O/IPAc) with different a_{w} at RT and 50° C. Similar mass of Form G and Form I were added into a 2-mL vial, followed by adding solvent and stirring at RT or 50° C. for 24 hrs. The solids obtained were analyzed by XRPD to confirm the form change.

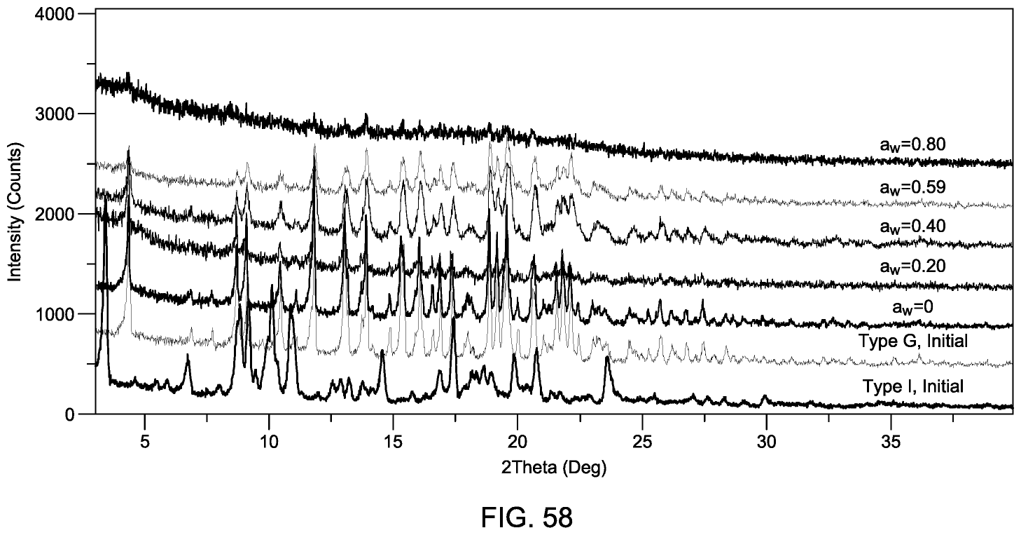

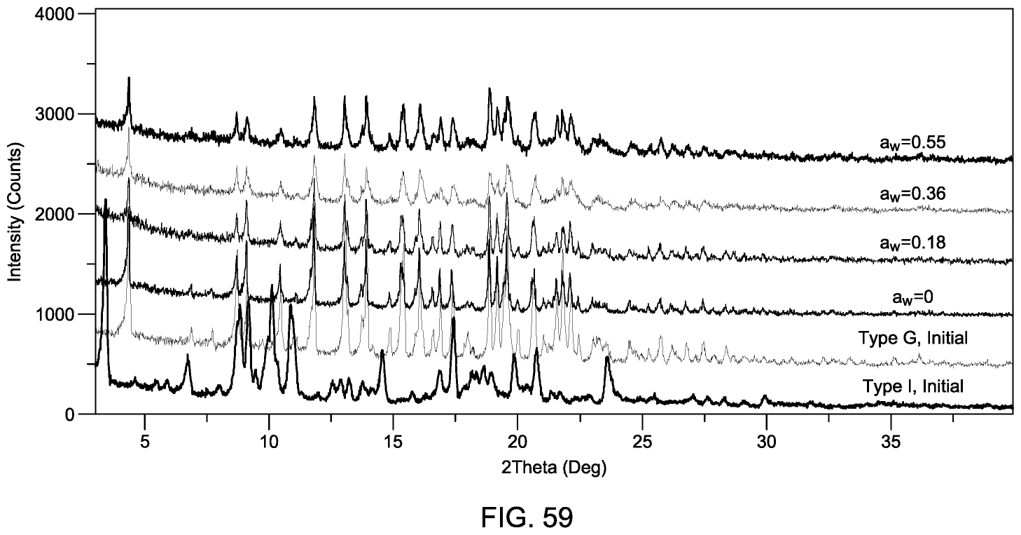

As summarized in Table 26 and FIGS. 58 and 59, the majority of the mixtures started in the slurries completely converted to Form G except the sample from the co-solvent with a_{w} of 0.8 at RT, which was close to an amorphous material.

TABLE 26

| Mass (mg) | | | | |
|---|---|---|---|---|
| Form I | Form G | Solvent (v:v, a_{w}) | Temp. | Final Form |
| 8.8 | 9.8 | H₂O/IPAc | RT | Mixture of |
| | | (15:985, 0.80) | | Form G and |
| | | | | amorphous |
| 9.9 | 10.0 | H₂O/IPAc | | Form G |
| | | (10:990, 0.59) | | |
| 10.3 | 10.4 | H₂O/IPAc | | Form G |
| | | (6:994, 0.40) | | |
| 9.9 | 10.1 | H₂O/IPAc | | Form G |
| | | (2.8:997.2, 0.20) | | |
| 10.3 | 10.2 | IPAc | | Form G |
| 6.9 | 7.1 | H₂O/IPAc | 50° C. | Form G |
| | | (10:990, 0.55) | | |
| 6.8 | 6.5 | H₂O/IPAc | | Form G |
| | | (6:994, 0.36) | | |
| 6.9 | 6.8 | H₂O/IPAc | | Form G |
| | | (2.8:997.2, 0.18) | | |
| 7.5 | 6.0 | IPAc | | Form G |

### Example 13—Solubility Estimation of Compound 2 Form G, Compound 2 Form O, and Compound 2 Form I

Aqueous solubility tests at RT were conducted on the two potential anhydrates, Form G and O, and the hydrated Form I. About 3 mg of each form was weighed into a 2-mL vial. 50 L of water was added and stirred thoroughly at RT with the speed of 800 RPM. After stirring for 2 hrs, clear solutions were obtained indicating that all the three forms show high solubility (>60 mg/mL) in water at RT (Table 27).

TABLE 27

| | | | |
|---|---|---|---|
| | | Volume | |
| Crystal | Mass | of water | Observation |

| Form | (mg) | (μL) | Initial | 2 hrs |
|---|---|---|---|---|
| Form G | 3.4 | 50 | Clear | Clear |
| Form I | 3.1 | 50 | Little solid | Clear |
| Form O | 3.1 | 50 | Little solid | Clear |

The forms were evaluated with fundamental techniques. With the current conversion data, Form G was shown to be the most stable form in IPAc and MTBE at RT or 50° C. The hydrate Form I did not appear to have any stability under ambient conditions compared to Form G with different a_{w}.

The two anhydrates, Form G and Form O, and Form I all showed high solubility (>60 mg/mL) in water.

Five forms (Form B, Form G, Form I, Form O and Form M) showed high hygroscopicity and became amorphous after DVS test.

### Example 14—Polymorph Screening of Compound 1

Polymorph screening was conducted under 28 conditions using Compound 1 as starting material. Four methods including slurry, evaporation, anti-solvent addition and solid vapor diffusion were used and no crystalline form was found from the screening.

Slurry experiments were conducted at 4° C., RT and 50° C. in different solvent systems. For each experiment about 100 mg of Compound 1 was suspended in 0.4-1.0 mL of solvent in a 1.5-mL glass vial. The suspension was then kept stirring for about one week at specific temperature before the remaining solids were collected for XRPD analysis. As summarized in Table 28, no crystalline form was observed.

TABLE 28

| Solvent, v/v | Temperature, ° C. | Crystalline Form |
|---|---|---|
| ACN | RT | No* |
| MEK/Heptane, 1:3 | RT | No |
| IPAc/Heptane, 1:3 | RT | No |
| THF/Heptane, 1:3 | RT | No |
| EtOH/H₂O, 1:5 | RT | No |
| H₂O | 50 | No |
| Heptane | 50 | No |
| ACN/H₂O, 3:1 | 4 | No* |
| Cyclopentyl methyl ether | 4 | No* |
| DCM | 4 | No* |
| MTBE | 4 | No* |
| DMF | 4 | No* |

*Clear solution obtained at 4° C. with stirring, and solids obtained from evaporation at RT

Evaporation experiments were conducted under nine conditions. For each experiment about 20 mg of Compound 1 was dissolved in ˜1.5 mL of solvent in a 1.8-mL glass vial. The resulting clear solution was then subjected to slow evaporation at RT to induce precipitation. The solids, if observed, were isolated for XRPD analysis. The results are summarized in Table 29. No crystalline form was discovered.

TABLE 29

| Solvent, v/v | Crystalline Form |
|---|---|
| ACN | No |
| Acetone | No |
| EtOAc | No |
| 1,4-Dioxane | No |
| IPA | No |
| MeOH | No |
| EtOH/H₂O, 1:1 | No |
| Toluene | No |
| 2-MeTHF | No |

A total of six anti-solvent addition experiments were conducted. About 20 mg of Compound 1 was dissolved in 0.1-0.3 mL solvent to obtain a near-saturated solution. 0.5-5.0 mL anti-solvent was then added to induce precipitation. The precipitate was isolated for XRPD analysis after stirring the resulting suspension overnight. Results summarized in Table 30 indicate that no crystalline form was observed.

TABLE 30

| Solvent, v/v | Anti-solvent | Crystalline Form |
|---|---|---|
| 2-MeTHF | H₂O | No |
| ACN | H₂O | No |
| DMSO | H₂O | No |
| IPAc | Heptane | No |
| DCM | Heptane | No |
| Toluene | Heptane | No |

For the solid vapor diffusion experiment conducted, about 30 mg of Compound 1 was weighed into a 3-mL vial, which was then placed into a 20-mL vial containing 4 mL of water. The 20-mL vial was sealed and kept at RT for about two weeks, allowing sufficient time for water vapor to interact with the solid sample. The solids thus obtained were isolated for XRPD test. No crystalline form was observed.

1. A salt form of Compound 1: 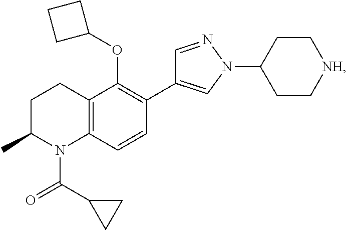 [SMILES: C[C@H]1CCc2c(ccc(-c3cnn(C4CCNCC4)c3)c2OC2CCC2)N1C(=O)C1CC1]
selected from the group consisting of a Compound 1 salt form of fumaric acid, adipic acid, and succinic acid.

2. The salt form claim 1, wherein the salt form is crystalline.

3. The salt form of claim 1 or 2, wherein Compound 1 is a salt of succinic acid (“Compound 1 Succinate”).

4. A crystalline solid form of Compound 2:

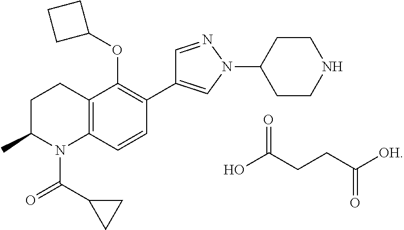

5. The solid form of claim 4, wherein the solid form is Solid Form G of (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate (“Compound 2”).

6. The solid form of claim 4 or 5, wherein Solid Form G of Compound 2 is characterized by an X-ray powder diffraction (XRPD) pattern having diffractions at angles (2 theta±0.2) of 4.4, 6.8, 9.1, 15.3, and 38.8.

7. The solid form of any one of claims 4-6, wherein Solid Form G of Compound 2 is characterized by an XRPD pattern having diffractions at angles (2 theta±0.2) of 4.4, 6.8, 9.1, 13.1, 15.3, 16.1, 19.6, 36.6, and 38.8.

8. The solid form of any one of claims 4-7, wherein Solid Form G of Compound 2 is characterized by an X-ray Powder Diffraction (XRPD), having diffractions at angles (2 theta±0.2) of 4.4, 6.8, 9.1, 13.1, 15.3, 16.1, 19.6, 36.6, and 38.8, corresponding to d-spacing (angstroms±0.2) of 20.1, 13.0, 9.7, 6.8, 5.8, 5.5, 4.5, 2.5, and 2.3 (respectively).

9. The solid form of any one of claims 4-8, wherein Solid Form G of Compound 2 is characterized by an XRPD pattern having diffractions at angles (2 theta±0.2) of:
4.4
6.8
7.7
8.7
9.1
10.5
11.1
11.8
13.1
13.9
14.9
15.3
16.1
16.6
16.9
17.4
18.0
18.2
18.9
19.2
19.6
20.0
20.7
21.1
21.6
21.8
22.1
22.5
23.0
23.5
24.5
25.3
25.8
26.2
26.8
27.5
27.9
28.4
28.7
29.1
29.9
31.0
32.7
33.3
34.0
35.2
36.1
36.6
38.8

10. The solid form of any one of claims 4-9, wherein Solid Form G of Compound 2 is characterized by an XRPD pattern having diffractions at angles (2 theta±0.2) corresponding to d-spacing (angstroms±0.2) of:

| 2 theta | d-spacing |
|---|---|
| 4.4 | 20.1 |
| 6.8 | 13.0 |
| 7.7 | 11.4 |
| 8.7 | 10.1 |
| 9.1 | 9.7 |
| 10.5 | 8.4 |
| 11.1 | 7.9 |
| 11.8 | 7.5 |
| 13.1 | 6.8 |
| 13.9 | 6.4 |
| 14.9 | 5.9 |
| 15.3 | 5.8 |
| 16.1 | 5.5 |
| 16.6 | 5.3 |
| 16.9 | 5.2 |
| 17.4 | 5.1 |
| 18.0 | 4.9 |
| 18.2 | 4.9 |
| 18.9 | 4.7 |
| 19.2 | 4.6 |
| 19.6 | 4.5 |
| 20.0 | 4.4 |
| 20.7 | 4.3 |
| 21.1 | 4.2 |
| 21.6 | 4.1 |
| 21.8 | 4.1 |
| 22.1 | 4.0 |
| 22.5 | 4.0 |
| 23.0 | 3.9 |
| 23.5 | 3.8 |
| 24.5 | 3.6 |
| 25.3 | 3.5 |
| 25.8 | 3.5 |
| 26.2 | 3.4 |
| 26.8 | 3.3 |
| 27.5 | 3.2 |
| 27.9 | 3.2 |
| 28.4 | 3.1 |
| 28.7 | 3.1 |
| 29.1 | 3.1 |
| 29.9 | 3.0 |
| 31.0 | 2.9 |
| 32.7 | 2.7 |
| 33.3 | 2.7 |
| 34.0 | 2.6 |
| 35.2 | 2.6 |
| 36.1 | 2.5 |
| 36.6 | 2.5 |
| 38.8 | 2.3 |

11. The solid form of any one of claims 4-10, wherein Solid Form G is characterized by a differential scanning calorimetry (DSC) endotherm having a minima at about 127° C.

12. The solid form of any one of claims 4-11, wherein Solid Form G is characterized by a thermogravimetric analysis (TGA) with a weight loss of about 0.2% between 21-150° C.

13. The solid form of any one of claims 4-12, wherein Solid Form G is characterized by a dynamic vapor sorption (DVS) of about 0.76% water by weight below 70% relative humidity.

14. A pharmaceutical composition comprising the salt form of claims 1-3 or the solid form of claims 4-13 and a pharmaceutically acceptable excipient.

15. The pharmaceutical composition of claim 14, wherein the pharmaceutical composition is for oral administration.

16. A method of inhibiting bromo and extra terminal (BET) bromodomains, comprising administering the salt form of claims 1-3 or the solid form of claims 4-13 to a subject.

17. A method of treating a disease, disorder, or condition responsive to inhibition BET, comprising administering the salt form of claims 1-3 or the solid form of claims 4-13 to a subject in need thereof.

18. The method of claim 17, wherein the disease, disorder, or condition is cancer.

19. A process for preparing Solid Form G of (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate (“Compound 2”) comprising suspending at least one of Form A, Form B, Form I, or Form O of Compound 2 in a solvent to provide a slurry, and maintaining the slurry for a period of time under conditions effective to generate Solid Form G of Compound 2.

20. The process of claim 19, wherein the solvent is selected from isopropyl acetate (IPAc), methyl tert-butyl ether (MTBE), methyl isobutyl ketone (MIBK), methylene chloride/methyl tert-butyl ether (CHCl₃/MTBE).

21. The process of claim 19 or 20, wherein the slurry is heated to a maximum temperature of about 50° C. after suspension in the solvent.

22. The process of any one of claims 19-21, further comprising isolating Solid Form G of Compound 2 from the slurry.

23. A process for preparing Solid Form G of (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate (“Compound 2”) comprising the step of contacting methyl tert-butyl ether with at least one of Form A, Form B, Form I, or Form O of Compound 2 in isopropyl acetate under conditions effective to generate Solid Form G of Compound 2.

24. A composition comprising methyl tert-butyl ether, isopropyl acetate, and at least one of Form A, Form B, Form I, or Form O of (S)-(5-cyclobutoxy-2-methyl-6-(1-(piperidin-4-yl)-1H-pyrazol-4-yl)-3,4-dihydroquinolin-1(2H)-yl)(cyclopropyl)methanone succinate.
